(19) United States
(12) Patent Application Publication
(10) Pub. No.: US 2024/0293356 A1
(43) Pub. Date: Sep. 5, 2024

(54) Cannabinoid Formulations

(71) Applicant: Endocanna Health, Inc., Burbank, CA (US)

(72) Inventors: Len May, Encino, CA (US); Eric Kaufman, Encino, CA (US)

(21) Appl. No.: 18/484,881

(22) Filed: Oct. 11, 2023

Related U.S. Application Data
(62) Division of application No. 16/760,159, filed on Apr. 29, 2020, now Pat. No. 11,857,530.
(60) Provisional application No. 62/707,300, filed on Oct. 30, 2017.
(60) Provisional application No. 62/680,885, filed on Jun. 5, 2018.
(60) Provisional application No. 62/721,294, filed on Aug. 22, 2018.

(51) Int. Cl. A61K 31/352; A61K 31/015; A61K 31/05; A61K 45/06; G06F 3/0484; G16B 20/20; G16H 10/40; G16H 10/60; G16H 15/00; G16H 20/10; G16H 50/20; G16H 50/70; G16H 70/40

(52) U.S. Cl. CPC A61K 31/352; A61K 31/015; A61K 31/05; G06F 3/0484; G16B 20/20; G16H 10/40; G16H 10/60; G16H 15/00; G16H 20/10; G16H 50/20; G16H 50/70; G16H 70/40; A61K 45/06

(57) ABSTRACT

The disclosure is directed to formulations comprising cannabinoids. More particularly, the cannabinoid formulations can be designed to align with an individual's genotype based on a defined list of polymorphisms.

### RELATED APPLICATIONS

This application claims the benefit of U.S. Provisional Application No. 62/707,300, filed October 30. 2017. U.S. Provisional Application No. 62/680,885. filed Jun. 5, 2018, and U.S. Provisional Application No. 62/721,294, filed Aug. 22, 2018, the disclosures of each of which are explicitly incorporated by reference herein in their entirety.

### FIELD OF THE INVENTION

The disclosure is directed to cannabinoid formulations. More particularly, the cannabinoid formulations can be designed to align with an individual's genotype based on a defined list of polymorphisms.

### BACKGROUND OF THE INVENTION

Medical use of Cannabis and associated phytocannabinoids is becoming widely accepted in the United States and Canada as an alternative form of medicine. Many states have legalized its use for qualified medical conditions such as chronic pain, epilepsy, sleep disorders, anxiety, cancer, glaucoma, nausea, ALS, Alzheimer's disease, Crohn's disease, Post-traumatic Stress Disorder (PTSD), arthritis, fibromyalgia, and others. Every individual has an endocannabinoid system comprised of chemical receptors in the brain, immune system and central nervous system (for example, cannabinoid receptors CB1 and CB2). Single nucleotide polymorphisms (SNPs) are stable genetic markers throughout the human genome, which can be tested for their association with various disease traits. These markers can also be associated with various traits that can determine an individual's sensitivity to certain compounds present in Cannabis such as cannabinoids, terpenes, nitrogenous compounds, flavonoids, non-cannabinoid phenols and other miscellaneous chemical constituents. These endogenous endocannabinoid SNP markers can be tested in a patient and used as biomarkers that may predict how a patient will react or respond to the metabolism of compounds such as delta-9-THC, cannabidiol, other cannabinoids, and terpinoids found in Cannabis. Furthermore, these biomarkers may suggest the best modality of treatment based on an individual's genetic profile and presence of specific enzymes or lack thereof that may result in negative side-effects from these compounds.

The invention provides cannabinoid formulations that are designed to align with an individual's genotype based on a defined list of polymorphisms.

### SUMMARY OF THE INVENTION

It is against the above background that the present invention provides certain advantages and advancements over the prior art. This disclosure relates to cannabinoid formulations that are designed to align with an individual's genotype based on a defined list of polymorphisms, and provide more reliable methods for achieving consistent and predictable results. The inventors have found that specific phytocannabinoid ratios, secreted plant based oils, and terpene profiles align with a subject's genotype.

These and other features and advantages of the present invention will be more fully understood from the following detailed description taken together with the accompanying claims. It is noted that the scope of the claims is defined by the recitations therein and not by the specific discussion of features and advantages set forth in the present description.

In an aspect, the disclosure provides formulations comprising a cannabidiol (CBD) and a tetrahydrocannabinol (THC), wherein the formulation has a CBD:THC ratio from about 50:1 to about 1:50. In some embodiments, the formulations further comprise at least one terpene selected from alpha-bisabolol, alpha-phellandrene, alpha-pinene, alpha-terpinene, beta-caryophyllene, beta-ocimene, beta-pinene, bisabolol, borneol, cadinene, camphene, camphor, cannabinoids, carene, caryophyllene oxide, cedrene, citral, citronellol, curcuminoids, cymene, delta-3-carene, eucalyptol, eugenol, fenchol, gamma-terpinene, geraniol, geranyl acetate, ginkgolides, guaiol, humulene, isoborneol, isopulegol, limonene, linalool, menthol, myrcene, nerol, nerolidol, ocimene, para-cymene, phytol, pinene, pulegone, sabinene, salvinorin, terpineol, terpinolene, theramine and valencene. In certain embodiments, the at least one terpene comprises a primary terpene and a secondary terpene. In some embodiments, the formulations comprise less than about 25%, about 20%, about 15%, about 10%, about 5%, about 4%, about 3%, about 2%, or about 1% terpenes.

In some embodiments, the formulation comprises a primary terpene that is myrcene and a secondary terpene that is linalool, and has a CBD:THC ratio of about 1:1 to about 1:5. In certain embodiments, the formulation comprises 5% myrcene and 3% linalool, and has a CBD:THC ratio of 1:1. In an embodiment, the formulation comprises 5% myrcene, 3% linalool, CBD, and is essentially free of THC.

In some embodiments, the formulation comprises a primary terpene that is pinene and a secondary terpene that is terpineol, and has a CBD:THC ratio of about 20:1 to about 1:1. In certain embodiments, the formulation comprises 5% pinene and 3% terpineol, and has a CBD:THC ratio of 4:1. In an embodiment, the formulation comprises 5% pinene, 3% terpineol, CBD, and is essentially free of THC.

In some embodiments, the formulation comprises a primary terpene that is limonene and a secondary terpene that is linalool, and has a CBD:THC ratio of about 20: 1 to about 2:1. In certain embodiments, the formulation comprises 5% limonene and 3% linalool, and has a CBD:THC ratio of 1:1. In an embodiment, the formulation comprises 5% limonene, 3% linalool, CBD, and is essentially free of THC.

In some embodiments, the formulation comprises a primary terpene that is limonene and a secondary terpene that is pinene, and has a CBD:THC ratio of about 1:1 to about 1:20. In certain embodiments, the formulation comprises 5% limonene and 3% pinene, and has a CBD:THC ratio of 1:2. In an embodiment, the formulation comprises 5% limonene, 3% pinene, CBD, and is essentially free of THC.

In some embodiments, the formulation comprises a primary terpene that is linalool and a secondary terpene that is borneol, and has a CBD:THC ratio of about 18:1 to about 4:1. In certain embodiments, the formulation comprises 5% linalool and 1% borneol, and has a CBD:THC ratio of 4:1. In an embodiment, the formulation comprises 5% linalool, 1% borneol, CBD, and is essentially free of THC.

In some embodiments, the formulation comprises a primary terpene that is beta-caryophyllene and a secondary terpene that is humulene, and has a CBD:THC ratio of about 1:1 to about 1:10. In certain embodiments, the formulation comprises 5% beta-caryophyllene and 3% humulene, and has a CBD:THC ratio of 1:1. In an embodiment, the formulation comprises 5% beta-caryophyllene, 3% humulene, CBD, and is essentially free of THC.

In some embodiments, the formulation comprises a primary terpene that is beta-caryophyllene and a secondary terpene that is myrcene, and has a CBD:THC ratio of about 1:1 to about 1:6. In certain embodiments, the formulation comprises 5% beta-caryophyllene and 5% myrcene, and has a CBD:THC ratio of 1:2. In an embodiment, the formulation comprises 5% beta-caryophyllene, 5% myrcene, CBD, and is essentially free of THC.

In some embodiments, the formulations comprise less than about 0.03% THC. In certain embodiments, the formulations are essentially free of THC.

In some embodiments, the formulation comprises a CBD:THC ratio of about 1:1 to about 1:3. In certain embodiments, the formulation comprises branched-chain amino acids, L-glutamine, piperine, magnesium stearate, MCC, and silicon dioxide, wherein the CBD:THC ratio is about 1:2. In an embodiment, the formulation comprises branched-chain amino acids, L-glutamine, piperine, magnesium stearate, MCC, silicon dioxide, CBD, and is essentially free of THC.

In some embodiments, the formulations as disclosed herein comprise from about 0.1 to about 100 mg/mL of the CBD. In other embodiments, the formulations as described herein comprise from about 0.1 to about 100 mg/mL of the THC.

In some embodiments, the formulations as described herein are formulated for buccal, dermal, intranasal, intravenous, nasal, ophthalmic, oral, sublingual, topical, or transdermal administration.

In some embodiments, the formulations as described herein further comprise cannabigerol (CBG), cannabinol (CBN), cannabidvarin (CBDV), cannabidiolic acid (CBDA), tetrahydrocannabinolic acid (THCA), or tetrahydrocannabivarin (THCV).

In some embodiments, the formulations as described herein further comprise black pepper, branched-chain amino acids (BCAA), cayenne, cedarwood, chamomile, coconut oil, geranium, ginger, ginger oil, glutamine, guava, juniper berry, lavender, lemon, lemon oil, lemongrass, lime, lime oil, orange, orange oil, mango, marjoram, menthol, mint, mint oil, peppermint, peppermint oil, piperine, geranium, rosemary, sandalwood, or tangerine.

In another aspect, the disclosure provides methods for treating a sleeping disorder. stress or depression, inflammation, anxiety, or for improving wellness or recovery comprising administering a pharmaceutically effective amount of any of the formulations as disclosed herein.

In yet another aspect, the disclosure provides methods for treating a subject comprising:
(a) determining the subject's DNA genotype;
(b) matching the subject with any of the formulations as disclosed herein based on the subject's DNA genotype; and
(c) administering a pharmaceutically effective amount of any of the formulations as disclosed herein to the subject.

In some embodiments of the method of treating, the subject's DNA genotype is assessed to identify one or more single nucleotide polymorphisms (SNPs) and a pharmaceutically effective amount of any of the formulations as disclosed herein is administered to the subject as disclosed in Table 10.

### DETAILED DESCRIPTION OF THE INVENTION

The disclosure involves compositions comprising selective formulations comprising cannabinoid molecules and terpenes that can be extracted from Cannabis plants. The cannabinoid formulations as disclosed herein are designed to selectively affect a subject based on the subject's personal genetics, which results in important insights into the subject's optimal Cannabis experience. Thus, the formulations can be personalized to comprise specific cannabinoid ratios and terpene profiles that help the subject achieve optimum results. The formulations disclosed herein provide numerous benefits and advantages, and allow a wide range of prevention, treatment, and management options for subjects based on their specific genotype.

All publications, patents and patent applications cited herein are hereby expressly incorporated by reference for all purposes.

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably.” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “and/or” is utilized to describe multiple components in combination or exclusive of one another. For example, “x, y, and/or z” can refer to “x” alone, “y” alone, “>” alone, “x, y, and z,” “(x and y) or z,” “x and (y or z),” or “x or y or 7,” As used herein, the terms “polynucleotide,” “nucleotide,” “oligonucleotide,” and “nucleic acid” can be used interchangeably to refer to nucleic acid comprising DNA, RNA, derivatives thereof, or combinations thereof.

In one aspect, the disclosure provides formulations comprising a cannabidiol (CBD) and a tetrahydrocannabinol (THC), wherein the formulation has a CBD:THC ratio from about 50:1 to about 1:50. In some embodiments, the CBD:THC ratio of the formulations described herein will be greater than or equal to 50:1, 45:1, 40:1, 35:1, 30:1, 25:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3; 1:4, 1:5, 1:6, 1:7, 1:8, 1:9, 1:10, 1:11, 1:12; 1:13, 1:14, 1:15, 1:16, 1:17, 1:18, 1:19, 1:20, 1:25, 1:30, 1:30, 1:35, 1:40, 1:45, 1:50, or lower. In certain embodiments, the formulations as disclosed herein do not contain THC, or are essentially free of THC.

In some embodiments, the formulations further comprise at least one terpene. Examples of terpenes include, but are not limited to: alpha-bisabolol, alpha-phellandrene, alpha-pinene, alpha-terpinene, beta-caryophyllene, beta-ocimene, beta-pinene, bisabolol, borneol, cadinene, camphene, camphor, cannabinoids, carene, caryophyllene oxide, cedrene, citral, citronellol, curcuminoids, cymene, delta-3-carene, eucalyptol, eugenol, fenchol, gamma-terpinene, geraniol, geranyl acetate, ginkgolides, guaiol, humulene, isoborneol, isopulegol, limonene, linalool, menthol, myrcene, nerol, nerolidol, ocimene, para-cymene, phytol, pinene, pulegone, sabinene, salvinorin, terpineol, terpinolene, theramine and valencene. In certain embodiments, the formulations comprise at least a primary terpene and a secondary terpene.

As used herein, the term “cannabinoid” refers to the chemical compounds found in Cannabis that mimic and supplement body biochemicals-endocannabinoids-that interact and ultimately control receptors found in every cell in the human body. Cannabinoids in Cannabis, especially THC and CBD, modulate the receptors CB1 and CB2, which are involved in the function of nearly every body system, disease and condition. The cannabinoid receptor type 1 (CB1 receptor), is primarily expressed in the brain, and is encoded by the gene CNR1. Mutations of this gene alter the endocannabinoid system and response to THC. Genetic variants are associated with an increased risk of anxiety, onset of paranoia, and addiction. The cannabinoid receptor type 2 (CB2 receptor), is encoded by the gene CNR2, and is primarily expressed in the periphery, but may also be expressed in the brain following neuroinflammatory conditions. Mutations of this gene are associated with difficulty in controlling pain, including neuropathic pain and diabetic neuropathy.

Over 600 cannabinoids have been identified, but only 4-8 tetrahydrocannabinol (Δ-8 THC). Δ-9) tetrahydrocannabinol (Δ-9 THC), and hydroxyl metabolites of those are psychoactive. Cannabinoids commonly found in Cannabis include, but are not limited to, CBC, CBCV, CBD, CBDA, CBDV, CBG, CBGV, CBL, CBN, CBV, THC, THCA, THCV.

The amount of each cannabinoid depends on the strain.

In some embodiments, the formulations as disclosed herein can further comprise cannabigerol (CBG), cannabinol (CBN), cannabidvarin (CBDV), cannabidiolic acid (CBDA), tetrahydrocannabinolic acid (THCA), or tetrahydrocannabivarin (THCV). In certain embodiments, the formulations as disclosed herein can comprise one or more of CBC, CBCV, CBD, CBDA, CBDV, CBG, CBGV, CBL, CBN, CBV, THC, THCA, and THCV.

Cannabichromene (CBC) is non-psychoactive and does not affect the psychoactivity of THC. CBC is typically more common in tropical Cannabis varieties. Effects include anti-inflammatory and analgesic.

Cannabichromevarin (CBCV) is a propyl cannabinoid, which means it has a propyl chain in its molecular structure. Instead of having a pentyl chain like its counterpart, cannabichromene (CBC), it branches off to have a propyl chain. CBCV could relieve seizures in children and infants.

Cannabidiol (CBD) is a non-psychoactive compound in Cannabis that has significant medical benefits. In the general population, high doses of CBD is expected to produce sedative and calming effects, while lower CBD doses have been shown to enhance mood.

Cannabidiolic acid (CBDA) is a cannabinoid found in raw Cannabis, meaning fresh flowers and leaves that are unheated. CBDA is decarboxylated to CBD with heat and light exposure.

Cannabidivarin (CBDV) is a non-psychoactive cannabinoid known for its anticonvulsant effects. Both cannabidivarin (CBDV) and cannabidiol (CBD) activate and desensitize transient receptor potential vanilloid 1 (TRPV1) channels in vitro and thus have a potential for the treatment of neuronal hyperexcitability.

Cannabigerol (CBG) is a non-psychoactive cannabinoid found in the fresh Cannabis plant. CBG is made by the decarboxylation of CBGA. CBG is a highly potent agonist for α2 adrenoceptor and a blocker of serotonin 5-HT1A receptor. This activity can decrease anxiety and muscle tension.

Cannabigerovarin (CBGV) is the propyl homologue of cannabigerol (CBG). CBGV is a potent inhibitor of LPI-induced GPR55 signaling. CBGV has been shown to be holding great potential for treating cancer. CBGV was shown to be cytostatic in leukemic cells and caused a simultaneous arrest at all phases of the cell cycle.

Cannabicyclol (CBL) is a non-psychotomimetic cannabinoid found in the Cannabis species. CBL is a degradative product like cannabinol. Light converts cannabichromene to CBL.

Cannabinol (CBN) is a non-psychoactive cannabinoid found in fresh Cannabis, usually in low amounts. Unlike other cannabinoids, CBN does not stem from cannabigerol (CBG). CBN is formed by decarboxylation of CBNA. CBN has exhibited pain relief properties.

Cannabivarin (CBV), also known as cannabivarol, is a non-psychoactive cannabinoid found in minor amounts in the hemp plant Cannabis sativa. It is an analog of cannabinol (CBN) with the side chain shortened by two methylene bridges (—CH2—). CBV is an oxidation product of tetrahydrocannabivarin (THCV, THV).

Tetrahydrocannabinol (THC) is the most well-known and most abundantly available cannabinoid in Cannabis plants. THC is also the component in Cannabis that is responsible for the psychoactive effects, or the “high.” Also known as delta-9-tetracannabinol, it was first isolated in 1964.

Tetrahydrocannabinolic acid (THCA) is a non-psychoactive cannabinoid found in raw Cannabis, meaning fresh flowers and leaves that are unheated. THCA is decarboxylated to psychoactive THC with heat and light exposure. This decarboxylation is what happens when one vaporizes or smokes flower.

Tetrahydrocannabivarin (THCV) is a non-psychoactive cannabinoid in Cannabis, and a precursor of THC and CBD. THCV acts like a CB1 antagonist, and may aid in weight reduction. THCV has also shown to be useful for glycemic control in patients with type 2 diabetes.

As used herein the terms “terpene” and “terpinoid” can be used interchangeably and refer to a large and diverse class of organic compounds, produced by a variety of plants. They are often strong smelling and may have a protective function. Terpenes are known to play a role in traditional herbal remedies and are under investigation for antibacterial, antineoplastic, and other pharmaceutical functions. Terpenes can act synergistically with cannabinoids to provide a therapeutic effect. Terpene can be acyclic, monocyclic, bicyclic, or multicyclic, and can be derived biosynthetically from units of isoprene, which has the molecular formula C₅H₈. The basic molecular formulae of terpenes are multiples of that, (C₅H₈)ₙ where n is the number of linked isoprene units. The isoprene units may be linked together “head to tail” to form linear chains or they may be arranged to form rings. Non-limiting examples of terpenes include hemiterpenes, monoterpenes, sesquiterpenes, diterpenes, sesterterpenes, triterpenes, sesquarterpenes, tetraterpenes, polyterpenes, and norisoprenoids. Examples of terpenes can include: alpha-bisabolol, alpha-phellandrene, alpha-pinene, alpha-terpinene, beta-caryophyllene, beta-ocimene, beta-pinene, bisabolol, borneol, cadinene, camphene, camphor, cannabinoids, careen, caryophyllene oxide, citral, citronellol, curcuminoids, delta-3-carene, eucalyptol, eugenol, fenchol, gamma-terpinene, geraniol, ginkgolides, humulene, limonene, linalool, menthol, myrcene, nerol, nerolidol, ocimene, para-cymene, phytol, pinene, pulegone, salvinorin, terpineol, terpinolene, or valencene.

Alpha-bisabolol, also known as levomenol, is a natural monocyclic sesquiterpene with a mild, floral, peppery odor. It has also been used for hundreds of years in cosmetics because of its perceived skin healing properties. Bisabolol has been effective at blocking the effects of mutagens on genetic integrity in liver cells. Bisabolol has also exhibited strong effects on Campylobacter which are involved in severe food poisoning. Furthermore, bisabolol has been associated with pain relief and neuroprotection, with possible uses in Parkinson's and Alzheimer's.

Alpha-phellandrene has an aroma that is described as herbaceous, citrus, peppery, minty, and slightly green/woody. Strains containing large amounts of alpha-phellandrene can often be identified by their exceptionally minty taste. Alpha-phellandrene can be naturally sourced from corn parsley (Ridolfia segetum) and the elemi tree (Canarium luzonicum). Alpha-phellandrene is absorbed easily through the skin, a quality that has made it a valued substance in the perfume industry. Alpha-phellandrene has a long history in traditional Chinese medicine as a treatment for digestive disorders. More recent research has suggested that alpha-phellandrene possesses anti-depressive effects.

Alpha-pinene is an alkene and contains a highly reactive four membered ring perpendicular to the main ring, prone to skeletal rearrangements. Both the − and +enantiomers are seen in nature—alpha pinene is mostly seen in European pines, and +alpha pinene mostly in North American pines. The racemic mixture is seen in eucalyptus and citrus oils. Pinene is used in nature with the ozone to condense aerosols that could harm the environment.

Beta-caryophyllene is an important terpene found in Cannabis. Beta-caryophyllene has been shown to act as a full agonist of the CB2-receptor, although it does not act on the CB1-receptor. It has also been shown to exert anti-inflammatory and analgesic effect. Beta-caryophyllene is a sesquiterpene, with a sweet, woody, spicy, clove-like smell. It is a unique terpene due to its large size and structure. Due to these properties, beta-caryophyllene is able to activate several receptors in the body, including CB2, which is usually activated most by CBD. Beta-caryophyllene has been shown to be an effective analgesic by regulating neuroinflammation and thermal hyperalgesia. Also as an antioxidant, beta-caryophyllene is effective as demonstrated by preventing lipid oxidation and scavenging other radicals. As an anti-inflammatory, beta-caryophyllene has been proven to mediate kidney inflammation and its side effects. Furthermore, beta-caryophyllene has been eluted to be a gastric-protective.

Bisabolol (also known as a-bisabolol or levomenol) is a fragrant chemical compound produced by the chamomile flower and other plants such as the candeia tree in Brazil. It is also produced by various Cannabis strains. While it has long been widely used in the cosmetics industry, bisabolol has more recently become the subject of research for the medical benefits it displays in Cannabis. Bisabolol's effects and benefits can include anti-inflammatory. anti-irritant, antioxidant, anti-microbial, and analgesic.

Borneol is a bicyclic monoterpene with a balsam, camphor, herbal, woody scent. Borneol is commonly used in Asian traditional medicine. Borneol has demonstrated more potent effects than the local anesthetic, lidocaine, and can also be eaten for its analgesic effects. Borneol has acted as an anticoagulant in stroke models, and alleviates the mechanisms of proinflammatory cytokines in general. The terpene has induced fungicidal activity against several species of fungi. In addition, borneol has elucidated to help drugs cross the blood brain barrier at a higher rate than without borneol.

Cadinenes are bicyclic sesquiterpenes. The term cadinene has sometimes also been used in a broad sense to refer to any sesquiterpene with the so-called cadalane (4-isopropyl-1,6-dimethyldecahydronaphthalene) carbon skeleton. Cadinene can be derived from that of the Cade juniper (Juniperus oxycedrus L.).

Camphene is a particularly pungent, herbaceous terpene that possesses a camphoraceous, cooling, woody aroma with notes of citrus and mint. In the 1900s

Camphene was well known for its use as a fuel source for lamps and as a component of turpentine. Research suggests that camphene may be able to decrease nociceptive pain. Recent studies show that camphene may have a future in treating lung inflammation due to its promising ability to increase cell viability and improve mitochondrial membrane potential while decreasing lipid peroxidation.

Camphor has a strong, characteristic scent that is often familiar to those who have been acquainted with mothballs or Vicks® Vaporub™. Isolated camphor can be naturally sourced from the camphor laurel ((Cinnamomum camphora), rosemary (Rosmarinus officinalis), and the East African camphorwood (Ocotea usambarensis). Camphor has a historic past-the Chinese used it to embalm bodies and produce pigments used as ink. In medieval times bags of camphor in its powder or crystalline form were worn to defend from illness. In modern medicine, camphor is an active ingredient in various nasal decongestants and chest rubs. This is likely due to its anti-spasmodic and decongestant properties. Camphor has also been promoted as a natural insecticide.

Carene or delta 3-carene is terpene found in basil, bell peppers, rosemary, and Cannabis that promotes the drying up of excess liquid and has anti-inflammatory effects. Side effects often associated with this terpene are dry mouth and red eyes. Carene has a pungent and pleasant earthy aroma that is piney in resemblance. Carene is a bicyclic monoterpene with a unique propanol ring; is has a sweet citrusy odor. Carene has been implicated in helping differentiate and stimulate calcium production in bone cells. The terpene is also effective as a toxin for mosquitos.

Caryophyllene oxide is an oxygenated terpinoid, usually a metabolic by product of caryophyllene. Its use as an antifungal is highly effective with certain species. In addition, caryophyllene oxide has also been indicated as an anticoagulant with platelets.

Citral exudes an aroma reminiscent of citrus (particularly lemon/lime). Isolated citral can be naturally sourced from lemon myrtle (Backhousia citriodora), lemongrass (Cymbopogon), lemon verbena (Aloysia citrodora), lemons, limes, and oranges. Citral's attractive scent has led it to be a favored ingredient in perfumes, soaps, and other cleaning products. Citral exhibits a gastro-protective effect (potentially useful for those suffering from gastrointestinal issues). It has been suggested that the inhalation of citral's fragrance may lead to normalized hormone levels, promoting homeostasis. Through this interaction with the body's neuroendocrine system, it is possible that citral may be used to treat those suffering from major depressive disorders.

Citronellol is a sweet, floral terpene with an aroma reminiscent of roses and citrus. Isolated citronellol can be naturally sourced from rose and geranium. Citronellol is used in perfumes, aromatherapy and has been approved by the FDA as safe for food use. A natural mosquito repellent and antimicrobial agent, citronellol is generally gentle when applied to the skin. Inhalation of citronellol may lead to deep sedation.

Eucalyptol, also known as 1,8 cineole, has a fresh, strong eucalyptus, camphoraceous, minty odor. It is a cyclic ether monoterpene. Eucalyptol has been popularly used on the skin. gums, or other topical areas. The terpene is toxic to several species of bacteria including Staphylococcus aureus. Further research has proven eucalyptol to be a potential treatment for Alzheimer's, as it lowered the inflammation caused by amyloid beta plaques. Eucalyptol is also an anti-inflammatory for sinuses and the digestive system. As an antioxidant, eucalyptol was effective at preventing lipid oxidation. In addition, eucalyptol has been effective in battling leukemia and colon cancer cells. Asthma remedies have also been used with eucalyptol.

Eugenol emits a strong, clove-like aroma. Isolated Eugenol can be naturally sourced from cloves (Syzygium aromaticum). wormwood (Artemisia absinthium), cinnamon (Cinnamomum verum), nutmeg (Myristica fragrans), and Japanese star anise (Illicium anisatum). Eugenol is a common additive in clove cigarettes. As it can have calming and anti-inflammation effects, it is popularly used in various perfumes and massage oils. Eugenol has antiseptic and anesthetic properties when applied topically. A combination of eugenol and tea tree oil is known to be effective in treating fungal infections.

Fenchol, or 1,3,3-trimethyl-2-norbornanol is a terpene and an isomer of borneol. The naturally occurring enantiopure (1R)-endo-(+)-fenchol is used extensively in perfumery. Fenchol is also, a scent from basil.

Gamma-terpinene has a sweetly herbaceous aroma augmented with notes of citrus. Isolated gamma-terpinene can be naturally sourced from tea tree (Melaleuca alternifolia) and Lippia multiflora. Gamma-terpinene is a major component of citrus essential oils. Its pleasant scent and taste has lead gamma-terpinene to be used widely as a flavor and fragrance additive in the cosmetic and food industries. Gamma-terpinene has exhibited antimicrobial properties and may be used to deflect a variety of human pathogens. Gamma-terpinene has also been noted as a promoter of antioxidant, anti-inflammatory, and antiproliferative activities.

Geraniol has a sweet, floral, fruity, rosy, waxy, citrus smell. The terpene has been known as a good mosquito repellent, but unfortunately attracts bees. Geraniol is toxic to bacteria and certain fungi. Further uses for the terpene include anti-inflammatory action. Also as a topical drug enhancer and anti-inflammatory, geraniol has proven useful.

Humulene is one of the main terpenes in hops, from which it gets its name. Humulene is also called alpha-caryophyllene. Like beta-caryophyllene, humulene is a cannabinoid and sesquiterpene, although it does not contain a cyclobutane ring. Humulene is a powerful anti-inflammatory and an anti-pain compound. It also has anti-cancer properties. Humulene is unique because, like THCV, it acts as an appetite suppressant, showing promise for weight loss treatments.

Limonene is a terpene found in certain strains of Cannabis that conveys a pleasant citrus aroma. Limonene has been shown to help with inflammation, anxiety, acid reflux, allergies and depression. Limonene has been shown to be a potent antidepressant and anti-anxiety treatment comparable to some traditional medicines. Limonene has also been inferred to be an anti-inflammatory, lowering or preventing key stages in the reaction. Limonene was elucidated in being a potential treatment for breast, prostate and pancreatic cancer.

Linalool is a minor terpene found in certain strains of Cannabis that is also produced in a variety of mints and herbs, including lavender. When combined with other major terpenes and cannabinoids, linalool may alleviate a number of conditions including pain, inflammation, depression, insomnia, anxiety and substance abuse. Linalool is a terpene alcohol with a citrusy, floral, sweet, bois de rose, woody, green, blueberry scent. Linalool is able to act on the opioidergic and cholinergic systems to relieve pain, a unique pathway for terpenes. Linalool also acts as an anticonvulsant, having similar effects to diazepam. Linalool has been used as a relaxant and as a treatment for anxiety for thousands of years. In scientific studies, linalool was proven to sedate mice and also mitigate anxiety.

Myrcene is the most commonly detected terpene in Cannabis, and is a monoterpene that is an important precursor to many terpenes. Myrcene is also widely identified in plants, such as cloves, hops, lemon grass, and bay. It has been associated with having sedative, muscle relaxant and hypnotic properties and is commonly used for aromatic therapy of stress-relief. Myrcene is hypothesized to help compounds enter cells through enhancing membrane permeation. Myrcene has been shown to be an analgesic in mice and did not cause tolerance to the effect. Myrcene is also noted to have antioxidant effects with mutagenic compounds. Another benefit to myrcene is its ability to relax muscles and induce sleep.

Nerol is a strong, fresh terpene with a sweet rose aroma. Isolated nerol can be naturally sourced from neroli (Citrus aurantium), lemongrass (Cymbopogon citratus), and hops (Humulus lupulus). Nerol is used for its fragrance in perfumes and cosmetics and as a flavor agent in the food industry. Nerol may be used as a sedative, spasmolytic agent, and vasodilator. Nerol also possesses antiviral qualities.

Nerolidol is a sesquiterpene and that smells similar to fresh bark. There are two isomers present in nature, cis and trans. The terpene has been eluted to be a toxin against harmful protozoa like malaria and leishmaniasis. Furthermore, nerolidol is effect in delivering drugs through the skin.

Ocimene and beta-ocimene is a monoterpene with a fruity, floral, some say wet cloth smell. Ocimene has exhibited anti-inflammatory effects in white blood cell through a variety of pathways. Antifungal effects are also seen with the human specific Candida species. And very interestingly ocimene showed specificity and effectiveness against SARS virus.

Para-cymene is a constituent of a number of essential oils, most commonly the oil of cumin and thyme. Significant amounts are formed in sulfite pulping process from the wood terpenes.

Phytol is a mild floral terpene with a green, slightly balsamic aroma. Isolated phytol can be naturally sourced from barley (Hordeum vulgare L.) and green tea (Camellia sinensis). Phytol is a breakdown product in the decomposition of chlorophyll and acts as a precursor in the formation of vitamins E and K. Phytol plays an essential role in the human body by activating enzymes responsible for the production of insulin, and thereby helping to regulate blood glucose and cholesterol levels. Studies have shown that phytol may relieve insomnia, especially when used in conjunction with the terpene linalool. Phytol may also be used topically to reduce itchiness and treat wounds due to its anti-inflammatory and pain relieving qualities.

Pinene consists of two isomers, alpha and beta, depending on the position of the double bond within the molecule. Pinene is a terpene in Cannabis that carries the aroma of pine. It is also present in orange peels, basil, pine needles and parsley. Pinene may help with asthma and anxiety, while reducing inflammation, enhancing relaxation and improving focus. Pulegone emits a minty, faintly camphoraceous aroma. Isolated pulegone can be naturally sourced from pennyroyal (Mentha pulegium), catnip (Nepeta cataria), and peppermint (Mentha piperita). Pulegone is used by the candy and fragrance industries for its pleasant peppermint-like odor and flavor. Preservation and enhancement of memory is one of the effects of pulegone. This is due to pulegone's inhibition of the protein acetylcholinesterase. Thus, the memory is less affected by THC when it consumed with pulegone.

Terpineol has a citrusy, lime aroma with hints of lilac and apple blossom. Isolated terpineol can be naturally sourced from pine trees (Pinus), cajuput (Melaleuca cajuputi), and petitgrain (Citrus aurantium). Terpineol is most often used therapeutically to help manage pain and inflammation, and to reduce the frequency of seizures. Other medicinal values of terpineol include gastroprotection and promotion of antibacterial activities. Inhalation of terpineol may lead to a deep sedation.

Terpinolene is a terpene with a complex smoky, floral, herbal, or woody odor, commonly used in perfumes and soaps. It is naturally found in Cannabis as well as other pleasantly fragrant plants including nutmeg, lilac, tea tree and apples. Terpinolene aids with sleep and may also be used to help fight bacterial or fungal infections. Terpinolene is a common terpene primarily isolated from trees. Terpinolene also goes by the name delta-terpinene. Terpinolene is not an analgesic or an anti-inflammatory, yet most cannabinoids and terpenoids are one of the two or both. Terpinolene was concluded to be effective against several species of bacteria. Terpinolene is able to increase total antioxidant capacity levels in white blood cells without changing the total oxidative stress level. Terpinolene is further effective in fighting glial cell cancer and leukemia.

Valencene is a bicyclic sesquiterpene with a sweet, fresh, citrusy, grapefruit, woody, orange odor. The terpene has been shown to be toxic and repel ticks and mosquitoes at lesser concentrations than DEET and without the toxicity to humans. Valencene has also been deduced to be and anti-inflammatory, lower the levels of inflammatory markers in macrophages.

As used herein, the term “primary terpene” refers to a terpene that is the most abundant terpene in a formulation either in absolute content as a % by dry weight, or in relative content as a % of the terpene in each formulation. For example, in a formulation comprising 5% limonene and 3% pinene, limonene would be the primary terpene.

As used herein, the term “secondary terpene” refers to a terpene that is the second most abundant terpene in a formulation either in absolute content as a % by dry weight, or in relative content as a % of the terpene in each formulation. For example, in a formulation comprising 5% limonene and 3% pinene, pinene would be the secondary terpene.

In an embodiment, the disclosure provides a formulation wherein the primary terpene is myrcene and the secondary terpene is linalool, and having a CBD:THC ratio of about 1:1 to about 1:5. In certain embodiments, the formulation comprises 5% myrcene, 3% linalool, and has a CBD:THC ratio of 1:1. In an embodiment, this formulation is essentially free of THC. In some embodiments, the disclosure relates to a method of treating a sleeping disorder by administering a pharmaceutically effective amount of the formulation.

Cannabis may help one fall asleep faster due its sleep-promoting effects. THC has well characterized sedative properties in rodents and humans. THC was also able to improve sleep in patients with obstructive sleep apnea and is being studied in restless legs syndrome. THC (and CBD) may also improve sleep indirectly by relieving symptoms of other conditions that make sleeping difficult, including pain, anxiety, PTSD, and multiple sclerosis. One study showed improved sleep quality in neuropathic pain patients after treatment with a mix of THC and CBD. CBD increased awake activity during sleep. For this reason, low CBD ratios are recommended near bedtime. However, CBD was also noted to have promise for excessive daytime sleepiness. CBN is a cannabinoid produced by degradation of THC through sunlight, heat and dry conditions. CBN is reported to have stronger sedative effects than THC itself.

Myrcene is a sedating terpene which is recognized by its musky, skunky smell. Its sedative and relaxing effects make it ideal for the treatment of insomnia and pain. Some contend that myrcene is the main Cannabis terpene contributing to “couch-lock”. Studies in rodents have shown that myrcene has several properties associated with sedatives, such as slowed motor activity, increased muscle relaxation, and increased sleeping time. Myrcene also exhibited anti-convulsant effects. Myrcene appears to work by enhancing activity of the inhibitory GABAA receptor. This mechanism is shared with benzodiazepines, which are sedative drugs used for anxiety and sleep. Some have proposed that myrcene works synergistically with THC to increase its sedative properties.

Studies in rodents have shown that myrcene has several properties associated with sedatives like for example prenobarbytal, which is evidenced by induction of P-450 (P-450) 2B subfamily) enzyme (see PubMed ID (PMID): 8257941), slowed motor activity, increased muscle relaxation, and increased sleeping time. Myrcene also exhibited anti-convulsant effects. Recently, it was shown in rodent models that administration of β-myrcene protects oxidative and histological damage in the heart tissue after global ischemia-reperfusion and may be useful safe alternative treatment for cardiac tissue after ischemic stroke (see PMID: 27487280). It can also exert fungistatic and fungicidal activities and serve as potent antioxidant (see PMID: 28680993; PMID: 21245202)

Myrcene appears to work by enhancing activity of the inhibitory GABAA receptor. This mechanism is shared with benzodiazepines, which are sedative drugs used for anxiety and sleep. Myrcene has a potential to be used as an analgesic, without developing tolerance to it. It appears to be a novel pain medication, without side effects of aspirin-like drugs, but more in human studies are needed (see PMID: 1753786). Some have proposed that myrcene works synergistically with THC to increase its sedative properties. Although, caution is warranted because high doses of myrcene might be anxiogenic (see PMID: 12587690).

Linalool may lessen the anxious emotions provoked by pure THC. thus making it helpful in the treatment of anxiety and other THC side effects. Linalool possesses anxiolytic and antidressant properties (see PMID: 26151006; PMID: 25771248). Studies in rodents have demonstrated the sedative effects of linalool, including decreased anxiety and reduced motor activity without a loss of coordination. Linalool also increased sleep and had anticonvulsant properties. Linalool may work in several ways. First, it is an antagonist of the NMDA receptor. This means that it can reduce activation of this receptor by glutamate, the main excitatory neurotransmitter of the brain. Other NMDA antagonists also have strong sedative properties. Second, it enhances GABAA activity, although it does this in a different way than myrcene since it does not bind the same receptor site. Based on the data in animal models, it might also possess antihypertensive properties, prevent development of cardiac hypertrophy, increase levels of the anti-inflammatory cytokine (IL-10), increase vasodilator responsiveness and reduce sensitivity to the sympathetic agonist (see PMID: 29454617). It has a beneficial potential for weight management and weight loss (see PMID: 29321988). Linalool may work in several ways. First, it is an antagonist of the NMDA receptor. This means that it can reduce activation of this receptor by glutamate, the main excitatory neurotransmitter of the brain. Other NMDA antagonists also have strong sedative properties. Second, it enhances GABA_{A} activity, although it does this in a different way than myrcene since it does not bind the same receptor site.

In another embodiment, the disclosure provides a formulation wherein the primary terpene is pinene and the secondary terpene is terpineol, and having a CBD:THC ratio of about 20:1 to about 1:1. In certain embodiments, the formulation comprises 5% pinene, 3% terpineol, and has CBD:THC ratio of 4:1. In an embodiment, this formulation is essentially free of THC. In some embodiments, the disclosure relates to a method of improving concentration by administering a pharmaceutically effective amount of the formulation.

THC may cause temporary impairments in memory and concentration. For this reason, low THC ratios are recommended when one needs to concentrate. Interestingly, a low dose THC was able to boost brain levels of the neurotransmitter acetylcholine. Low THC doses could also stimulate neurogenesis, which was linked to cognitive improvements. CBD can reduce the impact of THC on memory. Although studies in humans are needed. CBD could reverse some hyperactivity patterns in rats. CBD can reduce the impact of THC on memory. More research on this topic is summarized in: PMID: 29432803; PMID: 29098186; PMID: 25799920; and PMID: 27811555.

Pinene is a reported to add a unique dimension to the personality of certain Cannabis strains. Many strains with pinene as the dominant terpene are reported to promote alertness. Although the science behind the effects of pinene are not fully established, it is possible that pinene may improve memory through its antioxidant effects or boosting brain levels of acetylcholine. Acetylcholine is important for memory and cognition, but its release is decreased by THC. Through inhibition of acetylcholine metabolism, pinene and some other terpenes may improve cognitive impairment from THC. Pinene also had antidepressant effects in rodents, with mechanisms involving the serotonergic, adrenergic, and dopaminergic systems.

Terpineol can add a calming element that those with hyperactivity may find useful. Terpineol reduced locomotor activity in mice. Like some of the other Cannabis terpenes, terpineol can enhance activity of the inhibitory GABAA receptor. Terpineol also possesses pain reducing properties, anti-nociceptive/reducing pain (see PMID: 29380385).

In yet another embodiment, the disclosure provides a formulation wherein the primary terpene is limonene and the secondary terpene is linalool, and having a CBD:THC ratio of about 20): 1 to about 2:1. In certain embodiments, the formulation comprises 5% limonene and 3% linalool, and has a CBD:THC ratio of 1:1. In an embodiment, this formulation is essentially free of THC. In some embodiments, the disclosure relates to a method for treating stress or depression by administering a pharmaceutically effective amount of the formulation.

One of the important functions of the endocannabinoid system is adaptation to stress. Chronic stress is a risk factor for development and progression of depression and anxiety. THC can reduce anxiety, although it can also increase anxiety if the dose is too high. THC could be beneficial for certain disorders worsened by stress, such as migraines. In rodents, THC also showed antidepressant effects. CBD has shown anti-anxiety effects in humans, although studies are still ongoing. The mechanism appears to be both through the serotonin system and through boosting levels of endocannabinoids in the brain. CBD also showed antidepressant effects in rodents, although this has not been confirmed in humans. CBD could reduce certain side effects of THC, such as the psychotomimetic effects that occur in some people.

Limonene could contribute to an increase in attention span, mental focus and overall well-being. In rodents, limonene had anti-anxiety, anti-stress, and anti-depressant effects. Studies in rodents have demonstrated that linalool can decrease anxiety and reduce motor activity without a loss of coordination. In rat models with methamphetamine treatment, limonene reverses the increase in dopamine levels in the nucleus accumbens. These results suggest that limonene may inhibit stimulant-induced behavioral changes via regulating dopamine levels and 5-HT receptor function (see PMID: 24462212). S-limonene could also inhibit HPA reactivity under stress through the GABA(A) receptor (see PMID: 19763039), and exhibit anti-depressants properties (see PMID: 24661285).

During testing on the effects of limonene, participants experienced an increase in attention, mental focus, well-being and even libido. There are also undergoing trials for using limonene to treat depression and anxiety (see PMID:22364736; PMID:24125633; www.sciencedirect.com/topics/pharmacology-toxicology-and-pharmaceutical-science/limonene; PMID: 23554130; PMC4670880; PMC3165946; www.oncologyreviews.org/article/view/oncol.2011.31; PMID: 25388013; and PMID: 24160248).

Linalool may increase sleep and possess anticonvulsant properties. Linalool may lessen the anxious emotions provoked by pure THC, thus making it helpful in the treatment of anxiety and other THC side effects. Linalool also possesses antidepressant properties, possibly through effects on the serotonin system. Linalool may work in several ways. First, it is an antagonist of the NMDA receptor. This means that it can reduce activation of this receptor by glutamate, the main excitatory neurotransmitter of the brain. Other NMDA antagonists also have strong sedative properties. Second, it enhances GABAA activity, although it does this in a different way than myrcene since it does not bind the same receptor site.

In an embodiment, the disclosure provides a formulation wherein the primary terpene is limonene and the secondary terpene is pinene, and having a CBD:THC ratio of about 1:1 to about 1:20. In certain embodiments, the formulation comprises 5% limonene and 3% pinene, and has a CBD:THC ratio of 1:2. In an embodiment, this formulation is essentially free of THC. In some embodiments, the disclosure relates to a method for treating fatigue by administering a pharmaceutically effective amount of the formulation.

CBD has been proposed as a treatment for excessive daytime sleepiness. Cannabis is also being tested in fibromyalgia, which includes chronic fatigue as a symptom. Although so far there is limited evidence of the efficacy of pure THC, some studies have shown improvements with medical Cannabis.

Rodent studies indicate that limonene may reduce stress, anxiety, and depression, all of which can contribute to fatigue. Stress system activation was reduced by limonene through actions on the GABA system. Limonene may also have some regulation of the dopamine and serotonin systems, which are important for energy and motivation. Pinene is a reported to add a unique dimension to the personality of certain Cannabis strains. Many strains with pinene as the dominant terpene are reported to promote alertness. High levels of pinene were associated with an excitatory effect in the brain. Pinene may also promote alertness through antioxidant effects or boosting brain levels of acetylcholine. However, controlled studies are needed in humans to confirm these effects.

In another embodiment, the disclosure provides a formulation wherein the primary terpene is linalool and the secondary terpene is borneol, and having a CBD:THC ratio of about 18:1 to about 4:1. In certain embodiments, the formulation comprises 5% linalool and 1% borneol, and has a CBD:THC ratio of 4:1. In some embodiments, the disclosure relates to a method for treating anxiety by administering a pharmaceutically effective amount of the formulation.

The relationship between THC and anxiety is complicated. Most people are aware that over-consuming THC is capable of causing anxiety and even panic. However, THC can reduce stress-induced anxiety when consumed at the appropriate dose. CBD is capable of reducing anxiety, both after a single dose and when consumed over time. CBD works in several different ways to counteract anxiety. CBD can have a rapid effect through activation of a type of serotonin receptor called the 5-HT1A receptor. CBD can also boost levels of naturally occurring endocannabinoids in the brain such as anandamide. Over time, anandamide can stimulate neurogenesis in certain parts of a subject's brain. This has been linked to improvements in anxiety and stress resilience. CBD is also able to reduce anxiety generated by THC.

Linalool has been used for centuries as a sleep aid. Linalool may lessen the anxious emotions provoked by pure THC, thus making it helpful in the treatment of anxiety and other THC side effects. Studies in rodents have demonstrated the sedative effects of linalool, including decreased anxiety and reduced motor activity without a loss of coordination. Linalool also increased sleep and had anticonvulsant properties. Linalool may work in several ways. First, it is an antagonist of the NMDA receptor. This means that it can reduce activation of this receptor by glutamate, the main excitatory neurotransmitter of the brain. Other NMDA antagonists also have strong sedative properties. Second, it enhances GABAA activity, although it does this in a different way than myrcene since it does not bind the same receptor site.

β-caryophyllene is the only Cannabis terpene known to interact directly with the endocannabinoid system. It is a full agonist of the cannabinoid CB2 receptor. This means that β-caryophyllene is able to fully activate the CB2 receptor, whereas THC is generally only capable of partial activation. The CB2 receptor is present in many cell types throughout the body, but there are particularly high levels in immune cells. Activation does not result in an intoxicating effect, but it is an important regulator of inflammation and pain. CB2 activation resulted in improvement in several animal models of diseases, such as stroke, multiple sclerosis. Parkinson's disease, alcoholic liver disease, asthma, irritable bowel syndrome, and both neuropathic and inflammatory pain.

Borneol (also referred to as moxa) is a terpene in Cannabis that can be used to relieve pain, reduce inflammation, lower anxiety, neuroprotectant, antioxidant, and treat heart disease. A study completed in 2013 (www.hindawi.com/journals/tswj/2013/808460/abs/) showed that this terpene produces a significant reduction of nociceptive pain while also displaying anti-inflammatory activity in mice. Unlike other medications used to treat pain and inflammation, borneol did not impair motor coordination. Another study from 2003also confirmed that borneol can be used as a topical to numb pain and may be as effective as lidocaine (PMID: 12473382). In addition to reducing pain and inflammation, borneol can be used to manage anxiety.

In yet another embodiment, the disclosure provides a formulation wherein the primary terpene is beta-caryophyllene and the secondary terpene is humulene, and having a CBD:THC ratio of about 1:1 to about 1:10. In certain embodiments, the formulation comprises 5% beta-caryophyllene and 3% humulene, and has a CBD:THC ratio of 1:1. In an embodiment, this formulation is essentially free of THC. In some embodiments, the disclosure relates to a method for treating inflammation by administering a pharmaceutically effective amount of the formulation.

THC can activate both cannabinoid CB1 and CB2 receptors, which are expressed in many immune cells. Although CB2 is more commonly associated with an anti-inflammatory effect, CB1 also plays a role in activation of some immune cells. There are many studies demonstrating the anti-inflammatory properties of CBD. CBD general reduces activation of immune cells and has shown activity in many animal models of inflammatory and autoimmune conditions, including irritable bowel syndrome. The effect of CBD may involve activation of a type of intracellular receptors called PPARs, boosting endocannabinoid levels. or through other mechanisms. THC and CBD, both alone and in combination, have been tested in many studies of pain. This is a complex topic due to the number of different types of pain (inflammatory, neuropathic, migraine, etc.). Although there is still much to learn. these cannabinoids may be an effective way for some people to treat pain.

β-caryophyllene is the only Cannabis terpene known to interact directly with the endocannabinoid system. It is a full agonist of the cannabinoid CB2 receptor. This means that β-caryophyllene is able to fully activate the CB2 receptor, whereas THC is generally only capable of partial activation. The CB2 receptor is present in many cell types throughout the body, but there are particularly high levels in immune cells. Activation does not result in an intoxicating effect, but it is an important regulator of inflammation and pain. CB2 activation resulted in improvement in several animal models of diseases, such as stroke, multiple sclerosis. Parkinson's disease, alcoholic liver disease, asthma, irritable bowel syndrome, and both neuropathic and inflammatory pain. For example, CB2 activation has improved several animal models of diseases (see bmcneurol.biomedcentral.com/articles/10. 1186/1471-2377-6-12, such as stroke, multiple sclerosis. Parkinson's disease, alcoholic liver disease, asthma, irritable bowel syndrome. Inflamation onlinelibrary.wiley.com/doi/10.1111/j.1471-4159.2005.03380.x/full and both neuropathic and inflammatory pain; onlinelibrary.wiley.com/doi/10.1038/sj.bjp.0707505/full).

Humulene has been used in Chinese medicine for generations. Humulene possesses both anti-inflammatory and pain reducing properties, as well as immune boosting properties (see PMID: 17559833; herb.co/marijuana/news/humulene; PMID: 18053325; PMID: 12802719; and PMID: 19921589). The anti-cancer properties of humulene were first highlighted in a 2003 study (see PMID: 12802719), which suggests it may be a result of humulene's ability to produce Reactive Oxygen Species (ROS). ROS have various roles in cancer, contributing to the death of cancer cells through apoptosis at some levels; although at other levels ROS can actually increase the growth rate of tumors. Both ROS maximizing and ROS minimizing approaches have been developed and are commonly used, though ROS maximizing strategies seem to be more common. A demonstration of the entourage effect can be seen in a 2007 study (see PMID: 18053325), which showed that β-caryophyllene potentiates the anti-cancer effects of humulene.

In an embodiment, the disclosure provides a formulation wherein the primary terpene is beta-caryophyllene and the secondary terpene is myrcene, and having a CBD:THC ratio of about 1:1 to about 1:6. In certain embodiments, the formulation comprises 5% beta-caryophyllene and 5% myrcene, and has a CBD:THC ratio of 1:2. In an embodiment, this formulation is essentially free of THC. In some embodiments, the disclosure relates to a method for improving wellness by administering a pharmaceutically effective amount of the formulation.

β-caryophyllene is the only Cannabis terpene known to interact directly with the endocannabinoid system. It is a full agonist of the cannabinoid CB2 receptor. This means that β-caryophyllene is able to fully activate the CB2 receptor, whereas THC is generally only capable of partial activation. The CB2 receptor is present in many cell types throughout the body, but there are particularly high levels in immune cells. Activation does not result in an intoxicating effect, but it is an important regulator of inflammation and pain. CB2 activation resulted in improvement in several animal models of diseases, such as stroke, multiple sclerosis, Parkinson's disease, alcoholic liver disease, asthma, irritable bowel syndrome, and both neuropathic and inflammatory pain.

Myrcene is a sedating terpene which is recognized by its musky, skunky smell. Its sedative and relaxing effects make it ideal for the treatment of insomnia and pain. Some claim that myrcene is the main Cannabis terpene contributing to “couch-lock”. Studies in rodents have shown that myrcene has several properties associated with sedatives, such as slowed motor activity, increased muscle relaxation, and increased sleeping time. Myrcene also exhibited anti-convulsant effects. Myrcene appears to work by enhancing activity of the inhibitory GABAA receptor. This mechanism is shared with benzodiazepines, which are sedative drugs used for anxiety and sleep. Some have proposed that myrcene works synergistically with THC to increase its sedative properties.

In an embodiment, the disclosure provides a formulation comprising less than about 0.03% THC, and having a CBD:THC ratio of about 1:0.2 to about 1:0. In an embodiment, this formulation is essentially free of THC.

Cannabidiol, (CBD) is the major non-psychoactive component of Cannabis sativa. CBD activates 5-HT1A serotonin receptor, which helps with anxiety, addiction, appetite, sleep, nausea, vomiting. It also binds to TRPV1 receptors, which moderates perception of pain, inflammation, body temperature as well as blocks G protein receptor GPR55, which may decrease bone reabsorption and the spread of cancer cells. CBD activates peroxisome proliferator activated receptors (PPARs), which has been shown to produce anti-cancer effect and help with Alzheimer's. CBD benefits include acting in some experimental models as an anti-inflammatory, anticonvulsant, antioxidant, antiemetic, anxiolytic and antipsychotic agent, and is therefore a potential medicine for the treatment of neuro-inflammation, epilepsy, oxidative injury, vomiting and nausea, anxiety and schizophrenia.

In an embodiment, the disclosure provides a powder-based formulation that is encapsulated for oral administration, and having a CBD:THC ratio of about 1:1 to about 1:3. In certain embodiments, the formulation comprises branched-chain amino acids (BCAA: L-leucine, L-isoleucine, L-valine), L-glutamine, piperine (for example, BioPerine®). Magnesium stearate, MCC, or silicon dioxide. In an embodiment, the formulation has a CBD:THC ratio of about 1:2. In an embodiment, this formulation is essentially free of THC. In some embodiments, the disclosure relates to a method for improving recovery (for example, following a workout or exercise) by administering a pharmaceutically effective amount of the formulation.

Piperine may help with nutrient absorption through its ability to increase the level of absorption of nutrients (referred to as “bioenhancement”), and improve metabolism as well as immune function. Piperine may also help improve dopamine and serotonin levels, which can improve memory and mental skills

Branched-chain amino acids (BCAA) are amino acids having an aliphatic side-chain with a branch, and include leucine, isoleucine, and valine. BCAAs are among the essential amino acids for humans, accounting for 35% of the essential amino acids in muscle proteins and 40% of the preformed amino acids required by mammals. BCAAs fill several metabolic and physiologic roles. Metabolically. BCAAs promote protein synthesis and turnover, signaling pathways, and metabolism of glucose. Oxidation of BCAAs may increase fatty acid oxidation and play a role in obesity. Physiologically, BCAAs take on roles in the immune system and in brain function. BCAAs are broken down effectively by dehydrogenase and decarboxylase enzymes expressed by immune cells, and are required for lymphocyte growth and proliferation and cytotoxic T lymphocyte activity. Lastly, BCAAs share the same transport protein into the brain with aromatic amino acids (Trp, Tyr, and Phe). Once in the brain BCAAs may have a role in protein synthesis, synthesis of neurotransmitters, and production of energy.

In some embodiments of the formulations as disclosed herein, the formulations can comprise less than about 25%, about 20%, about 15%, about 10%, about 5%, about 4%, about 3%, or about 2% or less of any one terpene. In certain embodiments, the formulations as disclosed herein can comprise 0%, 0.01%, 0.02%, 0.04%, 0.06%, 0.08%, 0.10%, 0.12%, 0.14%, 0.16%, 0.18%, 0.20%, 0.22%, 0.24%, 0.26%, 0.28%, 0.30%, 0.32%, 0.34%, 0.36%, 0.38%, 0.40%, 0.42%, 0.44%, 0.46%, 0.48%, 0.50%, 0.52%, 0.54%, 0.56%, 0.58%, 0.60%, 0.62%, 0.64%, 0.66%, 0.68%, 0.70%, 0.72%, 0.74%, 0.76%, 0.78%, 0.80%, 0.82%, 0.84%, 0.86%, 0.88%, 0.90%, 0.92%, 0.94%, 0.96%, 0.98%, 1.0%, 1.02%, 1.04%, 1.06%, 1.08%, 1.10%, 1.12%, 1.14%, 1.16%, 1.18%, 1.20%, 1.22%, 1.24%, 1.26%, 1.28%, 1.30%, 1.32%, 1.34%, 1.36%, 1.38%, 1.40%, 1.42%, 1.44%, 1.46%, 1.48%, 1.5%, 1.6%, 1.7% 1.8%, 1.9%, 2.0%, 2.2%, 2.4%, 2.6%, 2.8%, 3%, 3.2%, 3.4%, 3.6%, 3.8%, 4.0%, 4.2%, 4.3%, 4.4%, 4.6%, 4.8%, 5.0%, 5.2%, 5.4%, 5.6%, 5.8%, 6.0%, 6.2%, 6.4%, 6.6%, 6.8%, 7.0%, 7.2%, 7.4%, 7.6%, 7.8%, 8.0%, 8.2%, 8.4%, 8.6%, 8.8%, 9.0%, 9.2%, 9.4%, 9.6%, 9.8%, 10.0%, 11.0%, 12.0%, 13.0%, 14.0%, 15.0%, 16.0%, 17.0%, 18.0%, 19.0%, 20.0%, 25.0%, or greater based on dry weight. In some embodiments the absolute content of any one terpene can be between about 0.01% and about 25.0%.

In some embodiments of the formulations as disclosed herein, the formulations can comprise from about 0.1 mg/mL to about 100 mg/mL of the CBD. In certain embodiments. the formulations as described herein comprise 0.01 mg/mL, 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.1 mg/mL, 2.2 mg/mL, 2.3 mg/mL, 2.4 mg/mL, 2.5 mg/mL, 2.6 mg/mL, 2.7 mg/mL, 2.8 mg/mL, 2.9 mg/ml, 3.0 mg/mL, 3.1 mg/mL, 3.2 mg/mL, 3.3 mg/mL, 3.4 mg/mL, 3.5 mg/mL, 3.6 mg/mL, 3.7 mg/mL, 3.8 mg/mL, 3.9 mg/mL, 4.0 mg/mL, 4.1 mg/mL, 4.2 mg/mL, 4.3 mg/mL, 4.4 mg/mL, 4.5 mg/mL, 4.6 mg/mL, 4.7 mg/mL, 4.8 mg/mL, 4.9 mg/mL, 5.0 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL or more of CBD. In some embodiments, the formulations as described herein comprise 0.01 g, 0.02 g, 0.03 g, 0.04 g, 0.05 g, 0.06 g, 0.07 g, 0.08 g, 0.09 g, 0.1 g, 0.2 g, 0.025 g, 0.3 g, 0.4 g, 0.5 g, 0.6 g, 0.7 g, 0.8 g, 0.9 g, 1.0 g, 1. 1 g, 1.2 g, 1.3 g, 1.4 g, 1.5 g, 1.6 g, 1.7 g, 1.8 g, 1.9 g, 2.0 g, 2. 1 g, 2.2 g, 2.3 g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4. 1 g, 4.2 g, 4.3 g, 4.4 g, 4.5 g, 4.6 g, 4.7 g, 4.8 g, 4.9 g, 5.0 g, 10 g, 15 g, 20 g, 25 g, 30 g, 35 g, 40 g, 45 g, 50 g, 55 g, 60 g, 65 g, 70 g, 75 g, 80 g, 85 g, 90 g, 95 g, 100 g, or more of CBD.

In some embodiments of the formulations as disclosed herein, the formulations can comprise from about 0.1 to about 100 mg/mL of the THC: however in certain embodiments the formulation may not contain THC, or are essentially free of THC. In certain embodiments, the formulations as described herein comprise 0.00 mg/mL, 0.01 mg/mL, 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.1 mg/mL, 2.2 mg/mL, 2.3 mg/mL, 2.4 mg/mL, 2.5 mg/mL, 2.6 mg/mL, 2.7 mg/mL, 2.8 mg/mL, 2.9 mg/mL, 3.0 mg/mL, 3. 1 mg/mL, 3.2 mg/mL, 3.3 mg/mL, 3.4 mg/mL, 3.5 mg/mL, 3.6 mg/mL, 3.7 mg/mL, 3.8 mg/mL, 3.9 mg/mL, 4.0 mg/mL, 4. 1 mg/mL, 4.2 mg/mL, 4.3 mg/mL, 4.4 mg/mL, 4.5 mg/mL, 4.6 mg/mL, 4.7 mg/mL, 4.8 mg/mL, 4.9 mg/mL, 5.0 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL or more of THC. In some embodiments, the formulations as described herein comprise, 0.00 g, 0.01 g, 0.02 g, 0.025 g, 0.03 g, 0.04 g, 0.05 g, 0.06 g, 0.07 g, 0.08 g, 0.09 g, 0.1 g, 0.2 g, 0.3 g, 0.4 g, 0.5 g, 0.6 g, 0.7 g, 0.8 g, 0.9 g, 1.0 g, 1.1 g, 1.2 g, 1.3 g, 1.4 g, 1.5 g, 1.6 g, 1.7 g, 1.8 g, 1.9 g, 2.0 g, 2.1 g, 2.2 g, 2.3 g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4.1 g, 4.2 g, 4.3 g, 4.4 g, 4.5 g, 4.6 g, 4.7 g, 4.8 g, 4.9 g, 5.0 g, 10 g, 15 g, 20 g, 25 g, 30 g, 35 g, 40 g, 45 g, 50 g, 55 g, 60 g, 65 g, 70 g, 75 g, 80 g, 85 g, 90 g, 95 g, 100 g, or more of THC.

A formulation is said to be “essentially free” of a particular component (i.e., THC, CBD, or a terpene), if that particular component is absent from the formulation or if the particular component is present in the formulation, but in an amount which is insufficient to promote any substantial effect in a subject, or is present at a concentration below a detectable limit. Additionally, it will be recognized that a formulation which is essentially free of a particular component may nonetheless contain trace amounts of the particular component in the formulation. For example, a formulation may contain no intentionally added THC and/or may contain no THC within conventional detection limits (thus, the term “essentially free of THC” encompasses the term “lacking THC”).

In certain embodiments of the formulations as disclosed herein, the formulations can comprise less than about 5%, about 4%, about 3%, or about 2%, or about 1% or less of any one or more of: black pepper, branched-chain amino acids (BCAA), cayenne, cedarwood, chamomile, coconut oil, geranium, ginger, ginger oil, glutamine, guava, juniper berry, lavender, lemon, lemon oil, lemongrass, lime, lime oil, orange, orange oil, mango, marjoram, menthol, mint, mint oil, peppermint, peppermint oil, piperine, geranium, rosemary, sandalwood, or tangerine. In certain embodiments, the formulations as disclosed herein can comprise about 0.01%, 0.02%, 0.04%, 0.06%, 0.08%, 0.10%, 0.12%, 0.14%, 0.16%, 0.18%, 0.20%, 0.25%, 0.30%, 0.40%, 0.50%, 0.60%, 0.70%, 0.80%, 0.90%, 1.0%, 1.1%, 1.2%, 1.3%, 1.4%, 1.5%, 1.6%, 1.7%, 1.8%, 2.0%, 2.2%, 2.4%, 2.6%, 2.8%, 3%, 3.2%, 3.4%, 3.6%, 3.8%, 4.0%, 4.2%, 4.3%, 4.4%, 4.6%, 4.8%, 5.0% or greater based on dry weight.

In some embodiments, the formulations are formulated for buccal, dermal, intranasal, intravenous, nasal, ophthalmic, oral, sublingual, topical, or transdermal administration. As different medical conditions can warrant different routes of administration, various forms for a particular formulation may exist. Methods for administration of the formulations described herein include, but are not limited, to inhalation (for example, dry powder inhalers, vaporizers, nebulizers, metered dose inhalers), smoking (e.g., dried buds), drinking, eating extracts or food products infused with concentrates or extracts, and taking capsules, Exemplary forms can include, but are not limited to, adhesive topical patch, aerosol, balm, capsule, chewing gum, cream, drops, elixir, emulsion, film, gas, gel granule, hydrogel, liniment, liquid, lollipop, lotion, lozenge, ointment, paste, pill, powder, skin patch, spray, strip, syrup, tablet, or tincture (a solvent extract of plant or animal material, or of a low volatility substance). For example, a composition formulated for oral administration can comprise a liquid gel capsule, a soft gel capsule, a tablet, a chewable tablet, a chewable wafer, an extended release formulation, a “gummy” candy (chewable lozenge: e.g. pectin or gelatin base), a lozenge, a pastille (e.g. polyol base), chewing gum, an effervescing tablet, or a liquid formulation, Gummies, lozenges, tablets, and capsules may, for example, be generated with sugar or as sugar free formulations.

In some embodiments, the formulations further comprise one or more of: binders, natural flavoring agents, artificial flavoring agents, disintegrants, emulsifiers, glidants (flow aids), granulating agents, natural colorants, artificial colorants, lubricants, preservatives, or sweetening agents. The formulations may include varied and numerous inactive ingredients known within the art to improve the formulation, delivery, preservation, appearance, palatability, and administration of the active ingredients.

In some embodiments, formulations are low in sugar (<40% sugar, less than about 30% or less than about 20% sugar), or are sugar-free. Sugar substitutes can include, but are not limited to, aspartame, sucralose, saccharin, stevia, monk fruit sweetener, erythritol, sorbitol, xylitol, mannitol, maltitol, and hydrogenated glucose or maltose syrups or maltodextrins, or combinations thereof.

Non-limiting examples of binders can include: acacia, tragacanth, gelatin, starch, cellulose based materials such as methyl cellulose and sodium carboxy-methyl cellulose, alginic acids and salts thereof, magnesium, aluminum silicate, polyethylene glycol, guar gum, polysaccharide acids, bentonites, sugars, invert sugars and the like.

Non-limiting examples coloring agents can include: known FD&C dyes and natural coloring agents such as grape skin extract, beet red powder, beta-carotene, annatto, carmine, turmeric, or paprika. The amount of coloring used may range from about 0.05 to about 5% by weight of the total composition.

Non-limiting examples of optional flavoring agents can include: synthetic flavors oils and flavoring aromatics, natural oils, plant extracts. Examples include almond oil, anise oil, avocado oil, bay oil, canola (rapeseed) oil, cedar leaf oil, cinnamon oil, coconut oil, clove oil, eucalyptus, flaxseed oil, grape seed oil, macadamia oil, nutmeg oil, olive oil, oil of wintergreen, peanut oil, peppermint oil, pine kernel oil, pomegranate seed oil, pumpkin seed oil, safflower oil, sesame oil, sage oil, soya bean oil, sunflower oil, thyme oil, omega 3 fatty acids (for example, ALA (alpha-linolenic acid), EPA (eicosapentaenoic acid) and DHA (docosahexaenoic acid)), omega 9 fatty acids, and combinations thereof. Examples of flavoring agents can also include, but are not limited to, almond, apple, banana, berry, bubblegum, caramel, citrus, cherry, chocolate, coconut, grape, green tea, honey, lemon, licorice, lime, mango, maple, mint, orange, peach, pineapple, raisin, strawberry, vanilla, watermelon, and combinations thereof. Flavoring agents may be present in an amount ranging from about 0.05 to about 5% by weight of the total composition.

Non-limiting examples of emulsifiers can include: lecithin, polysorbates, or sorbitan monooleates, and combinations thereof.

In certain embodiments, the formulations may further comprise an ingredient useful for increasing the storage stability of the formulations. In some embodiments this is an antioxidant. Suitable antioxidants can include molecules that inhibit the oxidation of other molecules. Non-limiting examples of antioxidants can include, but are not limited to, vitamin A, vitamin C, vitamin E, alpha-carotene, astaxanthin, beta-carotene, canthaxanthin, lutein, lycopene, zeaxanthin, flavonoids (such as apigenin, myricetin, eriodictyol, theaflavin, genistein, resveratrol, malvidin), cinnamic acid, chicoric acid, chlorogenic acid, rosmarinic acid, curcumin, xanthones, eugenol, citric acid, oxalic acid, and lipoic acid.

In an aspect, the disclosure relates to methods for treating a subject comprising: (a) determining the subject's DNA genotype; (b) matching the subject with a formulation as disclosed herein based on the subject's DNA genotype; and (c) administering a pharmaceutically effective amount of the formulation to the subject. In some embodiments of the method of treating a subject, the subject's DNA genotype is assessed to identify one or more single nucleotide polymorphisms (SNPs), and based on the SNP, a pharmaceutically effective amount of any of the formulations as disclosed herein is administered to the subject. A number of exemplary SNPs, with formulations, are discussed in Table 10.

In some embodiments, the formulations disclosed herein can be used to treat one or more of the following: acquired hypothyroidism, acute gastritis, agoraphobia, aids related illness, alcohol abuse, alcoholism, alopecia areata, Alzheimer's disease, amphetamine dependency, amyloidosis, amyotrophic lateral sclerosis (ALS), angina pectoris, ankylosis, ankylosing spondylitis, anorexia, anorexia nervosa, anxiety disorders, any chronic medical symptom that limits major life activities, any chronic medical symptom that limits major life activities, arteriosclerotic heart disease, arthritis, arthritis (rheumatoid), arthropathy, gout, asthma, attention deficit hyperactivity disorder (ADD/ADHD), autism, autoimmune disease, back pain, back sprain, Bell's palsy, bipolar disorder, brain tumor, malignant, bruxism, bulimia, cachexia, cancer, carpal tunnel syndrome, cerebral palsy, cervical disk disease, cervicobrachial syndrome, chemotherapy chronic fatigue syndrome, chronic pain, chronic renal failure, cocaine dependence, colitis, conjunctivitis, constipation, Crohn's disease, cystic fibrosis, damage to spinal cord nervous tissue, Darier's disease, degenerative arthritis, degenerative arthropathy, delirium tremens, dermatomyositis, diabetes, diabetic neuropathy, diabetic peripheral vascular disease, diarrhea, diverticulitis, dysthymic disorder, eczema, emphysema, emphysema, endometriosis, epidermolysis bullosa, epididymitis, epilepsy, Felty's syndrome, fibromyalgia, Friedreich's ataxia, gastritis, genital herpes, glaucoma, glioblastoma multiforme, Graves disease, cluster headaches, migraine headaches, tension headaches, hemophilia a, Henoch-Schonlein purpura, hepatitis C, hereditary spinal ataxia, HIV/AIDS, hospice patients, Huntington's disease, hypertension, hypertension, hyperventilation, hypoglycemia, impotence, inflammatory autoimmune-mediated arthritis, inflammatory bowel disease (IBD), insomnia, intermittent explosive disorder (IED), intractable pain, intractable vomiting, lipomatosis, Lou Gehrig's disease, lyme disease, lymphoma, major depression, malignant melanoma, mania, melorheostosis, Meniere's disease, motion sickness, mucopolysaccharidosis (MPS), multiple sclerosis (MS), muscle spasms, muscular dystrophy, myeloid leukemia, nail-patella syndrome, nightmares, obesity, obsessive compulsive disorder, opiate dependence, osteoarthritis, panic disorder, Parkinson's disease, peripheral neuropathy, peritoneal pain, persistent insomnia, porphyria, post-polio syndrome (PPS), post-traumatic arthritis, post-traumatic stress disorder (PTSD), premenstrual syndrome (PMS), prostatitis, psoriasis, pulmonary fibrosis, quadriplegia, radiation therapy, Raynaud's disease, Reiter's syndrome, restless legs syndrome (RLS), rheumatoid arthritis, rosacea, schizoaffective disorder, schizophrenia, scoliosis, sedative dependence, seizures, senile dementia, severe nausea, shingles (herpes zoster), sinusitis, skeletal muscular spasticity, sleep apnea, sleep disorders, spasticity, spinal stenosis, Sturge-Weber syndrome (SWS), stuttering, tardive dyskinesia (TD), temporomandibular joint disorder (TMJ), tenosynovitis, terminal illness, thyroiditis, tic douloureux, Tietze's syndrome, tinnitus, tobacco dependence, Tourette's syndrome, trichotillomania, viral hepatitis, wasting syndrome, whiplash, Wittmaack-Ekbom's syndrome, writers' cramp, nausea, vomiting, unintentional weight loss, insomnia, and lack of appetite, spasticity, painful conditions, especially neurogenic pain, movement disorders, asthma, glaucoma, adrenal disease, migraines, fibromyalgia, and related conditions, spinal cord injuries. The formulations disclosed herein can exhibit antispasmodic and muscle-relaxant properties as well as stimulates appetite, and may be useful in treating alcohol abuse, amyotrophic lateral sclerosis, collagen-induced arthritis, asthma, atherosclerosis, bipolar disorder, colorectal cancer, HIV-associated sensory neuropathy, depression, dystonia, epilepsy, digestive diseases, gliomas, hepatitis C, Huntington's disease, leukemia, skin tumors, methicillin-resistant Staphylococcus aureus (MRSA), Parkinson's disease, pruritus, posttraumatic stress disorder (PTSD), psoriasis, sickle-cell disease, sleep apnea, and anorexia nervosa. In other embodiments, the formulations as disclosed herein can be used for recreational purposes.

Anxiety is a common disorder experienced by many individuals. While the study behind the causes and effects of anxiety are ever changing, there have been several SNPs discovered that relate to the interaction of anxiety and cannabinoids. For example, the SNP rs1049353 for the CNR1 gene is associated with activation of specific brain areas (the insula and amygdala). The effect of this related to how one gauges visual, emotional, and social cues. Examples include facial expressions that change from anger to sadness or fear; happiness to sadness or fear; and neutral. Another example is the SNP rs324420 for the FAAH gene, which is associated with how an individual's endocannabinoid system (ECS) is related to specific neural mechanisms which may impact complex behavioral processes related to risk for addiction, dependence, and obesity. Yet another example is the SNP 5-HTTLPR for the SLC6A4 gene, which is associated with the development of anxiety for youth users of Cannabis. By analyzing these SNPs, a subject at risk for anxiety or suffering from anxiety may be effectively treated with the formulations as disclosed herein.

Bipolar disorder, or manic depression, is a serious brain illness that causes unusual shifts in mood, energy, activity, and the ability to carry out daily activities. Individuals suffering from bipolar disorder experience periods of intense emotion, changes in sleep, and unusual behavior, known as episodes. Episodes can be categorized as either manic (more energetic and “up” than normal) or depressive (more low energy and “down” than normal). While research surrounding bipolar disorder is ever changing, researchers have identified a SNP associated with the risk for developing bipolar disorder. For example, the SNP rs41311993 for the CNR2 gene is associated with the risk for developing bipolar disorder. By analyzing these SNPs, a subject at risk for developing bipolar disorder or suffering from bipolar disorder may be effectively treated with the formulations as disclosed herein.

Cognitive function may, for example, relate to a user's ability of their brain to process information and knowledge. While this is a general trait, researchers have identified SNPs that indicate how Cannabis may affect a user's cognitive function. In one example, the SNP rs1049353 for the CNR1 gene is associated with lower performance of executive function and sustained attention. Thus, depending on their endocannabinoid genotype, some users may experience an elevated risk of not being able to sustain attention when using Cannabis. In another example, the SNP rs4680 for the COMT gene is associated with risk of structural brain changes following Cannabis use. Users that have an at-risk genotype for this SNP (e.g., homozygous alleles, such as (A/A)), may want to consult with a specialist in cognitive function before using Cannabis. In yet another example, the SNP rs12199654 for the MAPK 14 gene is associated with a risk of decreased white matter brain volume from Cannabis use, which may result in impairing a user's cognitive function. In another example, the SNP rs7834206 for the NRGI gene is associated with auditory reception when using Cannabis. Users with heterozygous alleles (C/A) may be more likely to have auditory discrepancies after using Cannabis when compared to users with other genotypes. In yet another example, the SNP 5-HTTLPR for the SLC6A4 gene is associated with a user's focus, visual interpretation of their environment, and decision making. Users with homozygous alleles (L′/L′) might not experience a decrease in brain performance when using Cannabis. By analyzing these SNPs, the cognitive function of a subject may be effectively treated and/or improved with the formulations as disclosed herein.

Depression may, for example, relate to how a user feels, thinks, and acts. Specifically, depression is a long-term mental degradation that can affect the way a user functions in daily life. Depression can further be characterized by feeling tearful, irritable, and having diminished interest or pleasure in activities every day; significant weight loss/decrease or increase in appetite; inability to get to sleep or difficulty staying asleep or sleeping too much; problems with sitting or a slowing of one's movements; talking very quietly with slowed speech; fatigue; tiredness; feelings of worthlessness; diminished ability to think or concentrate; recurrent thoughts of death (not just fear of dying); recurrent suicidal ideas without a specific plan; or a suicide attempt or creating a specific plan for committing suicide. Due to the severity of depression symptoms, it is beneficial to have an understanding of how Cannabis may affect users that have a particular genotype. For example, the SNP rs1049353 for the CNR1 gene is associated with depression-specifically, how a user reacts to certain antidepressants such as citalopram. Users that have heterozygous alleles (C/T) may have a decrease likelihood of responding to antidepressants. In another example, the SNP rs2023239 for the CNR1 gene is associated with depression generally. Users that have homozygous alleles (T/T) may experience a higher likelihood of exacerbating pre-existing symptoms of depression when using Cannabis. In yet another example, the SNP rs806377 for the CNR1 gene is associated with how a user responds to positive emotional stimuli. Users with homozygous alleles (T/T) may experience a higher amount of positive emotions after a positive event than people with heterozygous alleles. In yet another example, the SNP rs324420) for the FAAH gene is associated with white matter integrity in the brain and increased reports of depression and apathy in Cannabis users. Users with homozygous alleles (CC) may experience decreased white matter in the brain and weakened brain structure when Cannabis is used at a young age. By analyzing these SNPs, a subject at risk for developing depression or suffering from depression may be effectively treated with the formulations as disclosed herein.

Impulsive behavior may, for example, relate to making decisions without thinking of the results and/or consequences beforehand. Impulsive behavior has many causes, which can include mental disorders such as hyperactivity disorder or personality disorders, such as borderline personality disorder. Cannabis usage may also cause impulsivity for certain users. For example, the SNP rs1049353 for the CNR1 gene is associated with adolescent psychosocial adversity, which is how one responds and/or adapts to family or relationship problems, health problems, school and other structural worries, and how they relate to impulsive behavior. Users with a genotype containing heterozygous alleles (C/T) may have an elevated risk of impulsive behavior when using Cannabis. In another example, the SNP rs806379 for the CNR1 gene is also associated with adolescent psychosocial adversity. Users with homozygous alleles (A/A) that experienced early psychosocial adversity may have a higher risk of impulsive behavior. In yet another example, the SNP rs1611115 for the DBH gene is associated with impulsivity after Cannabis consumption. Users with homozygous alleles (C/C) might not have increased impulsivity after Cannabis use, while users with heterozygous alleles may have increased impulsivity after Cannabis use. In yet another example, the SNP rs221533 for the NRGI gene is associated with lower inhibition and significantly riskier decision making. Users with heterozygous alleles (T/C) may have a lower risk of having behaviors associated with risky decision making when using Cannabis. In yet another example, the SNP rs28363170 for the SLC6A3 gene is also associated with impulsivity when using Cannabis. Users with homozygous alleles (10R/1OR) may have a lower risk of impulsivity after consuming Cannabis compared to users with heterozygous alleles. By analyzing these SNPs, a subject at risk for developing impulsive behavior or suffering from impulsive behavior may be effectively treated with the formulations as disclosed herein.

Memory impairment may, for example, relate to a person's ability to store information in their brain. For example, the SNP rs1049353 for the CNR1 gene is associated with varying brain awareness states, which is related to working memory ability and other cognitive functions. Users with heterozygous alleles (C/T) may have a normal state of awareness when compared to users with a different genotype. In another example, the SNP rs1406977 for the CNR1 gene is associated with performance on working memory tasks when using Cannabis. Users with homozygous alleles (T/T) may be less likely to experience working memory impairments after use of THC. By analyzing these SNPs, a subject at risk for developing memory impairment or suffering from memory impairment may be effectively treated with the formulations as disclosed herein.

Metabolic function may, for example, relate to how a user's cells breaks down materials from food to energy. Metabolic function may vary in users that are consuming Cannabis. For example, the SNP rs1045642 for the ABCB1 gene is associated with THC levels and THC metabolites in Cannabis users. Users with homozygous alleles (T/T) may have two-fold lower blood THC levels after consuming THC relative to people with a different genotype. In another example, the SNP rs1057910 for the CYP2C9 gene is associated with how oral THC is processed or metabolized in the body. Users with homozygous alleles (A/A) are typically no more sensitive to oral THC. By analyzing these SNPs, the metabolic function of a subject may be effectively treated and/or improved with the formulations as disclosed herein.

Migraines may, for example, relate to severe headaches that occur on one side of the head. Migraines can cause extreme discomfort and symptoms such as nausea and oversensitivity to lights and sounds. Research indicates that Cannabis usage may have an effect on migraines in certain individuals. For example, the SNP rs806366 for the CNRI gene is associated with a user's susceptibility to migraines. Users with homozygous alleles (T/T) may be more likely to develop migraines after stressful events. This is beneficial information because a medical provider can prescribe an appropriate dose if the provider is aware that the user is more likely to develop migraines. By analyzing these SNPs, a subject at risk for developing migraines or suffering from migraines may be effectively treated with the formulations as disclosed herein.

Motor control may, for example, relate to the process of creating and sending purposeful, voluntary movements throughout the body. Research indicates that the consumption of Cannabis may have profound effects on a user's motor control. For example, the SNP rs1130233 for the AKTI gene is associated with the degree of impairment in a user's psychomotor control and/or motor coordination after consumption of THC. Users with heterozygous alleles (C/T) may develop impaired motor coordination and slowed down thinking after consuming THC. By analyzing these SNPs, motor control in subject may be effectively treated and/or improved with the formulations as disclosed herein.

Opioids are, for example, a class of drugs created from the opium poppy plant. The plants are harvested and used in various types of medications because they contain a chemical that relaxes the body, and helps to relieve pain. Examples of opioids include Hydrocodone, Oxycodone, Oxymorphone, Morphine, Fentanyl, and Codeine. Research indicates that particular genetic markers may affect how a user reacts to opioids. For example, the SNP rs324420 for the FAAH gene is associated with having adverse opioid effects when combined with how a user's endocannabinoid system modulates, by way of such cannabinoids such as anandamide. Users with homozygous alleles (C/C) may have a lower risk of experienced side effects from opioids relative to people with a different genotype. By analyzing these SNPs, a subject at risk for developing opioid dependence or suffering from opioid dependence may be effectively treated with the formulations as disclosed herein.

Pain may, for example, relate to the unpleasant and corresponding emotional reaction in response to injury or tissue damage. Pain is a signal sent through the spinal cord, to a user's brain, alerting her that something is wrong in her body. Pain can be difficult to diagnose as it can manifest itself in different ways for different people. For example, the SNP rs324420 for the FAAH gene is associated with pain sensitivity and use of postoperative analgesia. Users with homozygous alleles (C/C) may have higher pain sensitivity to cold temperatures and more need for analgesia during periods of acute pain, such as after an operation. This information is beneficial when a provider is prescribing Cannabis after an operation. By analyzing these SNPs, a subject at risk for developing pain or suffering from pain may be effectively treated with the formulations as disclosed herein.

Psychosis may, for example, relate to a user's propensity for becoming disconnected from reality. Psychosis from Cannabis can cause delusions, which are strong beliefs that don't make sense and/or are not consistent with the user's actual beliefs. Research indicates that Cannabis may have a more profound effect on users with particular genetic markers. For example, the SNP rs1130233 for the AKTI gene is associated with the risk of psychosis-like effects (e.g., include delusion, delirium and confusion) after consuming THC. Users with heterozygous alleles (C/T) may have an increased risk of experience acute psychosis-like effects after consuming THC. In another example, the SNP rs2494732 for the AKTI gene is associated with the risk of psychotic episode in users that consume Cannabis. Users with homozygous alleles (T/T) may have a lower risk of experiencing psychotic disorder effects after consuming THC. In yet another example, the SNP rs6265 for the BDNF gene is associated with the onset of a psychotic disorder at a young age. Users with homozygous alleles (G/G) may not be at risk for onset psychosis if the user is already predisposed to developing psychosis. In yet another example, the SNP rs4680 for the COMT gene is also associated with psychosis-like effects (e.g., delusion, delirium, and confusion) after consuming THC. Users with homozygous alleles (A/A) may be less likely to experience psychosis-like effects after consuming THC relative to people with different genotypes. In another example, the SNP rs1076560 for the DRD2 gene is associated with a greater risk of developing psychosis (e.g., having regular hallucinations and delusions) in Cannabis users. In yet another example, the SNP rs2494732 for the AKTI gene is associated with a risk of a psychotic disorder and cognitive disabilities, including verbal memory and sustained attention impairments. Users with homozygous alleles (T/T) may have a lower risk of psychotic disorder and a lower risk of memory and attention impairments after consuming THC than users with a different genotype. This information may be particularly beneficial as it may prevent a user that is predisposed to psychosis from overdosing on THC. By analyzing these SNPs, a subject at risk for developing psychosis or suffering from psychosis may be effectively treated with the formulations as disclosed herein.

Psychotic like effects may, for example, include delusions and delirium caused by Cannabis usage. Research indicates that Cannabis use can cause schizophrenia, an illness that can cause a person to feel as if they have lost touch with reality. Research also indicates that certain genetic markers can indicate whether an individual is more likely to experience psychotic like effects when consuming Cannabis. For example, the SNP 5-HTTLPR for the gene SLC6A4 is associated with psychotic like effects in user with bipolar disorder when that user consumes Cannabis. By analyzing these SNPs, a subject at risk for developing psychotic like effects or suffering from psychotic like effects may be effectively treated with the formulations as disclosed herein.

Sleep quality may, for example, relate to the amount of time a user sleeps, the amount of times a user wakes up during the night, and the amount of time it takes a user to fall asleep. Research indicates certain genetic markers are related to sleep quality. For example, the SNP rs324420 for the FAAH gene is associated with poorer sleep quality among young Cannabis users who exhibit depression symptoms. Users with homozygous alleles (C/C) may have an increased risk of poor sleep quality while using certain cannabinoid formulations. By analyzing these SNPs, a subject's sleep quality may be effectively treated and/or improved with the formulations as disclosed herein.

In an embodiment of the methods disclosed herein, when the subject is determined to have heterozygous alleles (A/C) at the rs1057910 polymorphism of the CYP2C9 gene, the subject is administered a formulation wherein the primary terpene is linalool and the secondary terpene is beta-caryophyllene, and having a CBD:THC ratio of about 18:1 to about 4:1. This polymorphism was associated with metabolism and pharmacokinetics of oral THC. THC levels immediately after pulmonary administration (smoking, vaporizing) are not highly affected by the rate of metabolism, so this polymorphism probably has little effect with these methods. No effect on CBD levels is expected since it is not AC metabolized to a significant extent by CYP2C9. The subject may feel the effects of oral THC more strongly or find that the effects last longer relative to people with the most common genotype. It is recommended that the first time the subject uses oral THC, that the subject starts at 60% of the standard dose. No change in THC dose is needed for pulmonary administration.

In an embodiment of the methods disclosed herein, when the subject is determined to have heterozygous alleles (C/T) at the rs35599367 polymorphism of the CYP3A4 gene, the subject is administered subject is administered a formulation wherein the primary terpene is linalool and the secondary terpene is beta-caryophyllene, and having a CBD:THC ratio of about 18:1 to about 4:1. This polymorphism has not directly been associated with cannabinoid metabolism in a clinical study. However, it is associated with decreased expression and activity of the CYP3A4 enzyme. Clinical studies with the CYP3A4 inhibitor ketoconazole showed that it boosts both THC and CBD levels after oromucosal dosing by approximately 2-fold. Therefore, it is very likely that this polymorphism affects THC and CBD levels after oral dosing. A subject may feel the effects of oral THC more strongly or find that the effects last longer relative to other people. It is recommended that the first time a subject uses oral THC, that the subject start with a 1.5-fold lower dose. No change in THC dose is needed for pulmonary administration.

In an embodiment of the methods disclosed herein, when the subject is determined to have heterozygous alleles (C/T) at the rs1045642 polymorphism of the ABCB1 gene, the subject is administered subject is administered a formulation wherein the primary terpene is linalool and the secondary terpene is beta-caryophyllene, and having a CBD:THC ratio of about 18:1 to about 4:1. Genetic factors are known to influence Cannabis dependence. ABCB1 polymorphisms are known to modify drug pharmacokinetics and research studies have indicated their role in generating and maintaining Cannabis dependence. The biomarker rs1045642 has been identified and associated with the risk of Cannabis dependence. Research studies suggest that a subject may have a higher risk of Cannabis dependence relative to people with the TT genotype. Any subject should consult a specialist before titrating a dose of THC. Caution is warranted.

### EXAMPLES

The Examples that follow are illustrative of specific embodiments of the invention, and various uses thereof. They are set forth for explanatory purposes only, and are not to be taken as limiting the invention.

### Exemplary Formulations

1. 1:1 10 mg CBD, 10 mg THC (1 mg CBN, Myrcene 5%, Terpinolene 3%)
2. 4:1 40 mg CBD, 10 mg THC (α-Pinene 5%, Terpineol 3%)
3. 1:1 10 mg CBD, 10 mg THC (Limonene 5%, Linalool 3%, 1 mg Bisabolol)
4. 1:1 10 mg CBD, 10 mg THC (βn-Caryophyllene 5%, Limonene 3%)
5. 4:1 40 mg CBD, 10 mg THC (Linalool 5%, β-Caryophyllene, 1% Borneol, 1% Pinene)
6. 1:2 10 mg CBD, 20 mg THC (5% β-Caryophyllene, 3% Myrcene, 1% Humulene, 1% CBC)
7. 1:2 10 mg CBD, 20 mg THC (Limonene 5%, Pinene 3%, THCV 1%)

Additional exemplary formulations are shown in Tables 1-9.

For Tables 1-9, while the ratios and percentages remain constant, the number of milligrams for both Tetrahydrocannabinol (THC) and Cannabidiol (CBD) are variable. Because THC is the psychoactive component of these formulations, these models utilize the THC dose as the control. These models assume a defacto dose of 10 mg THC for THC-rich formulations and 1 mg THC for CBD-rich formulations. Defacto dose is assumed for individuals with normal metabolic function and without contraindications. Metabolic function can be indicated by pharmacogenomic testing and assessment of CYP-450 enzyme expression, for example. Dosing is incremental and can titrated up or down in these proportions.

TABLE 1
Exemplary formulations

| | EXEMPLARY | |
|---|---|---|
| FORMULATION | CONDITION(S) | COMPOSITION |
| UNWIND | Sleep apnea, | 1:1 to 1:5 CBD:THC |
| | Insomnia, | Myrcene 3.0-5.0% |
| | waking up easy | Linalool 1.5-5.0% |
| | | Terpinolene 1.0-3.0% |
| | | 0.04-0.1% Lavender |
| | | 0.04-0.1% Chamomile |
| | | 0.04-0.1% Sandalwood |
| FOCUS | ADHD, Autism, | 4:1 to 20:1 CBD:THC |
| | High energy, | alpha Pinene 3.0-5.0% |
| | Anxiety, | Terpineol 2.0-3.0% |
| | Hyperactivity | Eucalyptol 0.25-1.0% |
| | | 0.04-0.1% Wild Orange |
| | | 0.04-0.1% Peppermint |
| | | 0.04-0.1% Pulegone |
| COGNITIVE | Stress, | 1:1 to 20:1—CBD:THC |
| | Depression | Limonene 2.0-5.0% |
| | | Linalool 1.5-5.0% |
| | | 0.5-1.5% Bisabolol |
| | | 0.04-0.1% Juniper Berry |
| | | 0.04-0.1% Lime |
| | | 0.04-0.1% Lavender |
| IMMUNE | Auto-immune | 1:1 to 1:10—CBD:THC |
| | disorders, Pain, | Beta Caryophyllene 1.0-5.0% |
| | treatment of | Limonene 2.0-5.0% |
| | inflammation | 0.01-5.0% BCAA (branched |
| | related symptoms | chain amino acids) |
| | | 0.04-0.1% Glutamine |
| | | 0.04-0.1% Marjoram |
| | | 0.04-0.1% Ginger |
| | | 0.04-0.1% Lemongrass |
| RESPONSE | Fatigue, Low | 4:1 to 20:1—CBD:THC |
| | energy, Anxiety | Linalool 1.5-5.0% |
| | | Bomeol 0.5-2.0% |
| | | Beta Caryophyllene 1.0-5.0% |
| | | Pinene 3.0-5.0% |
| | | 0.04-0.1% Geranium |
| | | 0.04-0.1% Mint |
| | | 0.04-0.1% Guava |
| | | 0.04-0.1% Lavender |
| RELIEF | Cancer, Pain, | 1:1 to 1:20—CBD:THC |
| | Inflammation | Beta Caryophyllene 1.0-5.0% |
| | | Myrcene 3.0-5.0% |
| | | Humulene 1.0-2.0% |
| | | CBC 0.5-2.0% |
| | | 0.04-0.1% Lavender |
| | | 0.04-0.1% Sandalwood |
| | | 0.04-0.1% Cayenne |
| | | 0.04-0.1% Peppermint |
| ACTIVATE/ | Mood disorders, | 1:1 to 1:20—CBD:THC |
| WELLNESS | fatigue, low | Limonene 2.0-5.0% |
| | energy | Pinene 3.0-5.0% |
| | | THCV 0.5-2.0% |
| | | 0.04-0.1% Peppermint |
| | | 0.04-0.1% Eucalyptol |
| | | 0.04-0.1% Cedarwood |
| | | 0.04-0.1% Rosemary |
| RECOVERY | Post-workout, | 1:1 to 1:3—CBD:THC |
| | exercise | BCAA 500-1500 mg |
| | | L-glutamine 250-750 mg |
| | | Pipeline 1-10 mg |
| | | Magnesium stearate 5-15 mg |
| | | MCC (Endurance) 5-15 mg |
| | | Silicon dioxide 4-12 mg |

OPTIONAL INGREDIENTS: Any of the above formulations may also further comprise: CBC, CBCV, CBD, CBDA, CBDV, CBG, CBGV, CBL, CBN, CBV, THC, THCA, THCV, Ocimene, Valencene, Geraniol, Theramine, Phytol, Sabinene, Isobomeol, Cedrene, Guaiol, Geranyl Acetate, Eucalyptol, Carene, Fenchol, Bisabolol, Camphene, Camphor, Menthol, Nerolidol, Isopulegol, Cymene, or Pulegone.
In certain embodiments, any of the above formulations may be essentially free of THC.

Formulation #4
Wellness 1:1-1:20

| | THC Dose (mg): | | | | | | | | | |
|---|---|---|---|---|---|---|---|---|---|---|
| | 10 | 10 | 10 | 10 | 10 | 10 | 10 | 10 | 10 | 10 |
| Ratio: | 1:1 | 1:2 | 1:3 | 1:4 | 1:5 | 1:6 | 1:7 | 1:8 | 1:9 | 1:10 |
| By Milligrams: | | | | | | | | | | |
| CBD (mg) | 10.00 | 5.00 | 3.33 | 2.50 | 2.00 | 1.67 | 1.43 | 1.25 | 1.11 | 1.00 |
| THC (mg) | 10.00 | 10.00 | 10.00 | 10.00 | 10.00 | 10.00 | 10.00 | 10.00 | 10.00 | 10.00 |
| By Percentage: | | | | | | | | | | |
| CBD (%) | 50.0% | 33.33% | 25.00% | 20.00% | 16.67% | 14.29% | 12.50% | 11.11% | 10.00% | 9.09% |
| THC (%) | 50.0% | 66.67% | 75.00% | 80.00% | 83.33% | 85.71% | 87.50% | 88.89% | 90.00% | 90.91% |
| Additional | | | | | | | | | | |
| Cannabinoids, | | | | | | | | | | |
| Terpenes & | | | | | | | | | | |
| Active | | | | | | | | | | |
| Ingedients | | | | | | | | | | |
| Beta | 2.50% | 2.50% | 2.50% | 2.50% | 2.50% | 2.50% | 2.50% | 2.50% | 2.50% | 2.50% |
| Caryophylene | | | | | | | | | | |
| Limonene | 2.00% | 2.00% | 2.00% | 2.00% | 2.00% | 2.00% | 2.00% | 2.00% | 2.00% | 2.00% |
| Pinene | 1.00% | 1.00% | 1.00% | 1.00% | 1.00% | 1.00% | 1.00% | 1.00% | 1.00% | 1.00% |
| Majoram | 0.10% | 0.10% | 0.10% | 0.10% | 0.10% | 0.10% | 0.10% | 0.10% | 0.10% | 0.10% |
| Lemongrass | 0.10% | 0.10% | 0.10% | 0.10% | 0.10% | 0.10% | 0.10% | 0.10% | 0.10% | 0.10% |
| Mint | 0.10% | 0.10% | 0.10% | 0.10% | 0.10% | 0.10% | 0.10% | 0.10% | 0.10% | 0.10% |
| Ginger | 0.10% | 0.10% | 0.10% | 0.10% | 0.10% | 0.10% | 0.10% | 0.10% | 0.10% | 0.10% |
| | THC Dose (mg): | | | | | | | | | |
| | 10 | 10 | 10 | 10 | 10 | 10 | 10 | 10 | 10 | 10 |
| Ratio: | 1:11 | 1:12 | 1:13 | 1:14 | 1:15 | 1:16 | 1:17 | 1:18 | 1:19 | 1:20 |
| By Milligrams: | | | | | | | | | | |
| CBD (mg) | 0.91 | 0.83 | 0.77 | 0.71 | 0.67 | 0.63 | 0.59 | 0.56 | 0.53 | 0.50 |
| THC (mg) | 10.00 | 10.00 | 10.00 | 10.00 | 10.00 | 10.00 | 10.00 | 10.00 | 10.00 | 10.00 |
| By Percentage: | | | | | | | | | | |
| CBD (%) | 8.33% | 7.69% | 7.14% | 6.67% | 6.25% | 5.88% | 5.56% | 5.26% | 5.00% | 4.76% |
| THC (%) | 91.67% | 92.31% | 92.86% | 93.33% | 93.75% | 94.12% | 94.44% | 94.74% | 95.00% | 95.24% |
| Additional | | | | | | | | | | |
| Cannabinoids, | | | | | | | | | | |
| Terpenes & | | | | | | | | | | |
| Active | | | | | | | | | | |
| Ingedients | | | | | | | | | | |
| Beta | 2.50% | 2.50% | 2.50% | 2.50% | 2.50% | 2.50% | 2.50% | 2.50% | 2.50% | 2.50% |
| Caryophylene | | | | | | | | | | |
| Limonene | 2.00% | 2.00% | 2.00% | 2.00% | 2.00% | 2.00% | 2.00% | 2.00% | 2.00% | 2.00% |
| Pinene | 1.00% | 1.00% | 1.00% | 1.00% | 1.00% | 1.00% | 1.00% | 1.00% | 1.00% | 1.00% |
| Majoram | 0.10% | 0.10% | 0.10% | 0.10% | 0.10% | 0.10% | 0.10% | 0.10% | 0.10% | 0.10% |
| Lemongrass | 0.10% | 0.10% | 0.10% | 0.10% | 0.10% | 0.10% | 0.10% | 0.10% | 0.10% | 0.10% |
| Mint | 0.10% | 0.10% | 0.10% | 0.10% | 0.10% | 0.10% | 0.10% | 0.10% | 0.10% | 0.10% |
| Ginger | 0.10% | 0.10% | 0.10% | 0.10% | 0.10% | 0.10% | 0.10% | 0.10% | 0.10% | 0.10% |

Formulation #2
Focus 20:1-1:1

| | THC Dose (mg): | | | | | | | | | |
|---|---|---|---|---|---|---|---|---|---|---|
| | 1 | 1 | 1 | 1 | 1 | 1 | 1 | 1 | 1 | 1 |
| Ratio: | 20:1 | 19:1 | 18:1 | 17:1 | 16:1 | 15:1 | 14:1 | 13:1 | 12:1 | 11:1 |
| By Milligrams: | | | | | | | | | | |
| CBD (mg) | 20.00 | 19.00 | 18.00 | 17.00 | 16.00 | 15.00 | 14.00 | 13.00 | 12.00 | 11.00 |
| THC (mg) | 1.00 | 1.00 | 1.00 | 1.00 | 1.00 | 1.00 | 1.00 | 1.00 | 1.00 | 1.00 |
| By Percentage: | | | | | | | | | | |
| CBD (%) | 95.24% | 95.00% | 94.74% | 94.44% | 94.12% | 93.75% | 93.35% | 92.86% | 92.31% | 91.67% |
| THC (%) | 4.76% | 5.00% | 5.26% | 5.56% | 5.88% | 6.25% | 6.67% | 7.14% | 7.69% | 8.33% |
| Additional | | | | | | | | | | |
| Cannabinoids, | | | | | | | | | | |
| Terpenes & | | | | | | | | | | |
| Active | | | | | | | | | | |
| Ingedients | | | | | | | | | | |
| Terpenelene | 2.00% | 2.00% | 2.00% | 2.00% | 2.00% | 2.00% | 2.00% | 2.00% | 2.00% | 2.00% |
| Eucolyptol | 0.50% | 0.50% | 0.50% | 0.50% | 0.50% | 0.50% | 0.50% | 0.50% | 0.50% | 0.50% |
| Pinene | 3.00% | 3.00% | 3.00% | 3.00% | 3.00% | 3.00% | 3.00% | 3.00% | 3.00% | 3.00% |
| Wild Orange | 0.10% | 0.10% | 0.10% | 0.10% | 0.10% | 0.10% | 0.10% | 0.10% | 0.10% | 0.10% |
| Pulegone | 0.10% | 0.10% | 0.10% | 0.10% | 0.10% | 0.10% | 0.10% | 0.10% | 0.10% | 0.10% |
| Cayenne | 0.10% | 0.10% | 0.10% | 0.10% | 0.10% | 0.10% | 0.10% | 0.10% | 0.10% | 0.10% |
| Peppermint | 0.10% | 0.10% | 0.10% | 0.10% | 0.10% | 0.10% | 0.10% | 0.10% | 0.10% | 0.10% |
| | THC Dose (mg): | | | | | | | | | |
| | 1 | 1 | 1 | 1 | 1 | 1 | 1 | 1 | 1 | 1 |
| Ratio: | 10:1 | 9:1 | 8:1 | 7:1 | 6:1 | 5:1 | 4:1 | 3:1 | 2:1 | 1:1 |
| By Milligrams: | | | | | | | | | | |
| CBD (mg) | 10.00 | 9.00 | 8.00 | 7.00 | 6.00 | 5.00 | 4.00 | 3.00 | 2.00 | 10.00 |
| THC (mg) | 1.00 | 1.00 | 1.00 | 1.00 | 1.00 | 1.00 | 1.00 | 1.00 | 1.00 | 10.00 |
| By Percentage: | | | | | | | | | | |
| CBD (%) | 90.91% | 90.00% | 88.89% | 87.50% | 85.71% | 83.33% | 80.00% | 75.00% | 66.67% | 50.00% |
| THC (%) | 9.09% | 10.00% | 11.11% | 12.50% | 14.29% | 16.67% | 20.00% | 25.00% | 33.33% | 50.00% |
| Additional | | | | | | | | | | |
| Cannabinoids, | | | | | | | | | | |
| Terpenes & | | | | | | | | | | |
| Active | | | | | | | | | | |
| Ingedients | | | | | | | | | | |
| Terpenelene | 2.00% | 2.00% | 2.00% | 2.00% | 2.00% | 2.00% | 2.00% | 2.00% | 2.00% | 2.00% |
| Eucolyptol | 0.50% | 0.50% | 0.50% | 0.50% | 0.50% | 0.50% | 0.50% | 0.50% | 0.50% | 0.50% |
| Pinene | 3.00% | 3.00% | 3.00% | 3.00% | 3.00% | 3.00% | 3.00% | 3.00% | 3.00% | 3.00% |
| Wild Orange | 0.10% | 0.10% | 0.10% | 0.10% | 0.10% | 0.10% | 0.10% | 0.10% | 0.10% | 0.10% |
| Pulegone | 0.10% | 0.10% | 0.10% | 0.10% | 0.10% | 0.10% | 0.10% | 0.10% | 0.10% | 0.10% |
| Cayenne | 0.10% | 0.10% | 0.10% | 0.10% | 0.10% | 0.10% | 0.10% | 0.10% | 0.10% | 0.10% |
| Peppermint | 0.10% | 0.10% | 0.10% | 0.10% | 0.10% | 0.10% | 0.10% | 0.10% | 0.10% | 0.10% |

TABLE 4
Formulation #1
Unwind 1:1-1:5

| | | | | | |
|---|---|---|---|---|---|
| THC Dose | 10 | 10 | 10 | 10 | 10 |
| (mg): | | | | | |
| Ratio: | 1:1 | 1:2 | 1:3 | 1:4 | 1:5 |
| By Milligrams: | | | | | |
| CBD (mg) | 10.00 | 5.80 | 3.33 | 2.50 | 2.00 |
| THC (mg) | 10.00 | 10.00 | 10.00 | 10.00 | 10.00 |
| By percentage: | | | | | |
| CBD (%) | 50.00% | 33.33% | 25.00% | 20.00% | 16.67% |
| THC (%) | 50.00% | 66.67% | 75.00% | 80.00% | 83.33% |

Additional Cannabinoids, Terpenes & Active Ingredients

| | | | | | |
|---|---|---|---|---|---|
| Myecene | 3.50% | 3.50% | 3.50% | 3.50% | 3.50% |
| Linalool | 1.50% | 1.50% | 1.50% | 1.50% | 1.50% |
| Terpenolene | 2.50% | 2.50% | 2.50% | 2.50% | 2.50% |
| Lavender | 0.10% | 0.10% | 0.10% | 0.10% | 0.10% |
| Chamomile | 0.10% | 0.10% | 0.10% | 0.10% | 0.10% |
| Sandlewood | 0.10% | 0.10% | 0.10% | 0.10% | 0.10% |

TABLE 5
Formulation #3
Cognitive 20:1-2:1

| | THC Dose (mg): | | | | | | | | | |
|---|---|---|---|---|---|---|---|---|---|---|
| | 1 | 1 | 1 | 1 | 1 | 1 | 1 | 1 | 1 | 1 |
| Ratio: | 20:1 | 19:1 | 18:1 | 17:1 | 16:1 | 15:1 | 14:1 | 13:1 | 12:1 | 11:1 |
| By Milligrams: | | | | | | | | | | |
| CBD (mg) | 20.00 | 19.00 | 18.00 | 17.00 | 16.00 | 15.00 | 14.00 | 13.00 | 12.00 | 11.00 |
| THC (mg) | 1.00 | 1.00 | 1.00 | 1.00 | 1.00 | 1.00 | 1.00 | 1.00 | 1.00 | 1.00 |
| By Percentage: | | | | | | | | | | |
| CBD (%) | 95.24% | 95.00% | 94.74% | 94.44% | 94.12% | 93.75% | 93.35% | 92.86% | 92.31% | 91.67% |
| THC (%) | 4.76% | 5.00% | 5.26% | 5.56% | 5.88% | 6.25% | 6.67% | 7.14% | 7.69% | 8.33% |
| Additional | | | | | | | | | | |
| Cannabinoids, | | | | | | | | | | |
| Terpenes & | | | | | | | | | | |
| Active | | | | | | | | | | |
| Ingedients | | | | | | | | | | |
| Linalool | 2.00% | 2.00% | 2.00% | 2.00% | 2.00% | 2.00% | 2.00% | 2.00% | 2.00% | 2.00% |
| Limocene | 3.50% | 3.50% | 3.50% | 3.50% | 3.50% | 3.50% | 3.50% | 3.50% | 3.50% | 3.50% |
| Bisabolol | 1.00% | 1.00% | 1.00% | 1.00% | 1.00% | 1.00% | 1.00% | 1.00% | 1.00% | 1.00% |
| Lime | 0.10% | 0.10% | 0.10% | 0.10% | 0.10% | 0.10% | 0.10% | 0.10% | 0.10% | 0.10% |
| Junniper | 0.10% | 0.10% | 0.10% | 0.10% | 0.10% | 0.10% | 0.10% | 0.10% | 0.10% | 0.10% |
| Berry | | | | | | | | | | |
| Mint | 0.10% | 0.10% | 0.10% | 0.10% | 0.10% | 0.10% | 0.10% | 0.10% | 0.10% | 0.10% |
| Guava | 0.10% | 0.10% | 0.10% | 0.10% | 0.10% | 0.10% | 0.10% | 0.10% | 0.10% | 0.10% |
| Lavender | 0.10% | 0.10% | 0.10% | 0.10% | 0.10% | 0.10% | 0.10% | 0.10% | 0.10% | 0.10% |

| | THC Dose (mg): | | | | | | | | |
|---|---|---|---|---|---|---|---|---|---|
| | 1 | 1 | 1 | 1 | 1 | 1 | 1 | 1 | 1 |
| Ratio: | 10:1 | 9:1 | 8:1 | 7:1 | 6:1 | 5:1 | 4:1 | 3:1 | 2:1 |
| By Milligrams: | | | | | | | | | |
| CBD (mg) | 10.00 | 9.00 | 8.00 | 7.00 | 6.00 | 5.00 | 4.00 | 3.00 | 2.00 |
| THC (mg) | 1.00 | 1.00 | 1.00 | 1.00 | 1.00 | 1.00 | 1.00 | 1.00 | 1.00 |
| By Percentage: | | | | | | | | | |
| CBD (%) | 90.91% | 90.00% | 88.89% | 87.50% | 85.71% | 83.33% | 80.00% | 75.00% | 66.67% |
| THC (%) | 9.09% | 10.00% | 11.11% | 12.50% | 14.29% | 16.67% | 20.00% | 25.00% | 33.33% |
| Additional | | | | | | | | | |
| Cannabinoids, | | | | | | | | | |
| Terpenes & | | | | | | | | | |
| Active | | | | | | | | | |
| Ingedients | | | | | | | | | |
| Linalool | 2.00% | 2.00% | 2.00% | 2.00% | 2.00% | 2.00% | 2.00% | 2.00% | 2.00% |
| Limocene | 3.50% | 3.50% | 3.50% | 3.50% | 3.50% | 3.50% | 3.50% | 3.50% | 3.50% |
| Bisabolol | 1.00% | 1.00% | 1.00% | 1.00% | 1.00% | 1.00% | 1.00% | 1.00% | 1.00% |
| Lime | 0.10% | 0.10% | 0.10% | 0.10% | 0.10% | 0.10% | 0.10% | 0.10% | 0.10% |
| Junniper | 0.10% | 0.10% | 0.10% | 0.10% | 0.10% | 0.10% | 0.10% | 0.10% | 0.10% |
| Berry | | | | | | | | | |
| Mint | 0.10% | 0.10% | 0.10% | 0.10% | 0.10% | 0.10% | 0.10% | 0.10% | 0.10% |
| Guava | 0.10% | 0.10% | 0.10% | 0.10% | 0.10% | 0.10% | 0.10% | 0.10% | 0.10% |
| Lavender | 0.10% | 0.10% | 0.10% | 0.10% | 0.10% | 0.10% | 0.10% | 0.10% | 0.10% |

TABLE 6
Formulation #6
Response 18:1-4:1

| | THC Dose (mg): | | | | | | | |
|---|---|---|---|---|---|---|---|---|
| | 1 | 1 | 1 | 1 | 1 | 1 | 1 | 1 |
| Ratio: | 18:1 | 17:1 | 16:1 | 15:1 | 14:1 | 13:1 | 12:1 | 11:1 |
| By Milligrams: | | | | | | | | |
| CBD (mg) | 18.00 | 17.00 | 16.00 | 15.00 | 14.00 | 13.00 | 12.00 | 11.00 |
| THC (mg) | 1.00 | 1.00 | 1.00 | 1.00 | 1.00 | 1.00 | 1.00 | 1.00 |
| By Percentage: | | | | | | | | |
| CBD (%) | 94.74% | 94.44% | 94.12% | 93.75% | 93.33% | 92.86% | 92.31% | 91.67% |
| THC (%) | 5.26% | 5.56% | 5.88% | 6.25% | 6.67% | 7.14% | 7.69% | 8.33% |
| Additional | | | | | | | | |
| Cannabinoids, | | | | | | | | |
| Terpenes & | | | | | | | | |
| Active | | | | | | | | |
| Ingedients | | | | | | | | |
| Linalool | 3.50% | 3.50% | 3.50% | 3.50% | 3.50% | 3.50% | 3.50% | 3.50% |
| Terpenolene | 1.00% | 1.00% | 1.00% | 1.00% | 1.00% | 1.00% | 1.00% | 1.00% |
| Borneol | 2.00% | 2.00% | 2.00% | 2.00% | 2.00% | 2.00% | 2.00% | 2.00% |
| Mint | 0.10% | 0.10% | 0.10% | 0.10% | 0.10% | 0.10% | 0.10% | 0.10% |
| Guarava | 0.10% | 0.10% | 0.10% | 0.10% | 0.10% | 0.10% | 0.10% | 0.10% |
| Lavender | 0.04% | 0.04% | 0.04% | 0.04% | 0.04% | 0.04% | 0.04% | 0.04% |

| | THC Dose (mg): | | | | | | |
|---|---|---|---|---|---|---|---|
| | 1 | 1 | 1 | 1 | 1 | 1 | 1 |
| Ratio: | 10:1 | 9:1 | 8:1 | 7:1 | 6:1 | 5:1 | 4:1 |
| By Milligrams: | | | | | | | |
| CBD (mg) | 10.00 | 9.00 | 8.00 | 7.00 | 6.00 | 5.00 | 4.00 |
| THC (mg) | 1.00 | 1.00 | 1.00 | 1.00 | 1.00 | 1.00 | 1.00 |
| By Percentage: | | | | | | | |
| CBD (%) | 90.91% | 90.00% | 88.89% | 87.50% | 85.71% | 83.33% | 80.00% |
| THC (%) | 9.09% | 10.00% | 11.11% | 12.50% | 14.29% | 16.67% | 20.00% |
| Additional | | | | | | | |
| Cannabinoids, | | | | | | | |
| Terpenes & | | | | | | | |
| Active | | | | | | | |
| Ingedients | | | | | | | |
| Linalool | 3.50% | 3.50% | 3.50% | 3.50% | 3.50% | 3.50% | 3.50% |
| Terpenolene | 1.00% | 1.00% | 1.00% | 1.00% | 1.00% | 1.00% | 1.00% |
| Borneol | 2.00% | 2.00% | 2.00% | 2.00% | 2.00% | 2.00% | 2.00% |
| Mint | 0.10% | 0.10% | 0.10% | 0.10% | 0.10% | 0.10% | 0.10% |
| Guarava | 0.10% | 0.10% | 0.10% | 0.10% | 0.10% | 0.10% | 0.10% |
| Lavender | 0.04% | 0.04% | 0.04% | 0.04% | 0.04% | 0.04% | 0.04% |

TABLE 7
Formulation #7
Relief 1:1-1:6

| | THC Dose (mg): | | | | | |
|---|---|---|---|---|---|---|
| | 10 | 10 | 10 | 10 | 10 | 10 |
| Ratio: | 1:1 | 1:2 | 1:3 | 1:4 | 1:5 | 1:6 |
| By Milligrams: | | | | | | |
| CBD (mg) | 10.00 | 5.00 | 3.33 | 2.50 | 2.00 | 1.67 |
| THC (mg) | 10.00 | 10.00 | 10.00 | 10.00 | 10.00 | 10.00 |
| By Percentage: | | | | | | |
| CBD (%) | 50.00% | 33.33% | 25.00% | 20.00% | 16.67% | 14.29% |
| THC (%) | 50.00% | 66.67% | 75.00% | 80.00% | 83.33% | 85.71% |
| Additional | | | | | | |
| Cannabinoids, | | | | | | |
| Terpenes & | | | | | | |
| Active | | | | | | |
| Ingedients | | | | | | |
| Myrcene | 2.00% | 2.00% | 2.00% | 2.00% | 2.00% | 2.00% |
| Beta | 3.50% | 3.50% | 3.50% | 3.50% | 3.50% | 3.50% |
| Caryophyllene | | | | | | |
| Humulene | 1.00% | 1.00% | 1.00% | 1.00% | 1.00% | 1.00% |
| Lavender | 0.10% | 0.10% | 0.10% | 0.10% | 0.10% | 0.10% |
| Sandlewood | 0.10% | 0.10% | 0.10% | 0.10% | 0.10% | 0.10% |
| Pepperming | 0.10% | 0.10% | 0.10% | 0.10% | 0.10% | 0.10% |
| Rosemary | 0.10% | 0.10% | 0.10% | 0.10% | 0.10% | 0.10% |

TABLE 8
Formulation #5
IMMUNE 1:1-1:10

| | THC Dose (mg): | | | | | | | | | |
|---|---|---|---|---|---|---|---|---|---|---|
| | 10 | 10 | 10 | 10 | 10 | 10 | 10 | 10 | 10 | 10 |
| Ratio: | 1:1 | 1:2 | 1:3 | 1:4 | 1:5 | 1:6 | 1:7 | 1:8 | 1:9 | 1:10 |
| By Milligrams: | | | | | | | | | | |
| CBD (mg) | 10.00 | 5.00 | 3.33 | 2.50 | 2.00 | 1.67 | 1.43 | 1.25 | 1.11 | 1.00 |
| THC (mg) | 10.00 | 10.00 | 10.00 | 10.00 | 10.00 | 10.00 | 10.00 | 10.00 | 10.00 | 10.00 |
| By Percentage: | | | | | | | | | | |
| CBD (%) | 50.00% | 33.33% | 25.00% | 20.00% | 16.67% | 14.29% | 12.50% | 11.11% | 10.00% | 9.09% |
| THC (%) | 50.00% | 66.67% | 75.00% | 80.00% | 83.33% | 85.71% | 87.50% | 88.89% | 90.00% | 90.91% |
| Additional | | | | | | | | | | |
| Cannabinoids, | | | | | | | | | | |
| Terpenes & | | | | | | | | | | |
| Active | | | | | | | | | | |
| Ingedients | | | | | | | | | | |
| Beta | 3.00% | 3.00% | 3.00% | 3.00% | 3.00% | 3.00% | 3.00% | 3.00% | 3.00% | 3.00% |
| Caryophyllene | | | | | | | | | | |
| Humulene | 2.00% | 2.00% | 2.00% | 2.00% | 2.00% | 2.00% | 2.00% | 2.00% | 2.00% | 2.00% |
| Terpenolene | 1.00% | 1.00% | 1.00% | 1.00% | 1.00% | 1.00% | 1.00% | 1.00% | 1.00% | 1.00% |
| Lavender | 0.10% | 0.10% | 0.10% | 0.10% | 0.10% | 0.10% | 0.10% | 0.10% | 0.10% | 0.10% |
| Chamomile | 0.10% | 0.10% | 0.10% | 0.10% | 0.10% | 0.10% | 0.10% | 0.10% | 0.10% | 0.10% |
| Sandlewood | 0.10% | 0.10% | 0.10% | 0.10% | 0.10% | 0.10% | 0.10% | 0.10% | 0.10% | 0.10% |

TABLE 9
Formulation #8
Recovery (Capsules)
This product is a powder-based and
encapsulated for oral administration

| | | |
|---|---|---|
| Vegan Branch Chain Amino Acids, | 1,000 | mg |
| (L-Leucine, L-Isoleuice, L-Valline) | | |
| L-Glutamine | 500 | mg |
| BioPerine | 5 | mg |
| Magnesium Stearate | 10 | mg |
| MCC (Endurance) | 10 | mg |
| Silicon Dioxide (Sipernat22S) | 8 | mg |
| CBD/THC dose/ratio: | | |
| Hemp Extract (CBD)—5.5 mg | 5.5 | mg |
| THC Isolate Powder—2.5 mg | 2.5 | mg |

TABLE 10
Single-nucleotide polymorphisms (SNPs), endocannabinoid genotype, and potential formulation.

| | | | | | | References |
|---|---|---|---|---|---|---|
| SNIP I.D. | Gene | rsID | Category | Formulation | Effect/Findings/Discussion | (PMID) |
| 2-01- | ABCB1 | rs1045642 | Response to | RESPONSE | When consumed regularly, | 28917442 |
| AFMLG | | | Cannabinoids | | THC can accumulate in your | |
| | | | | | body. If for example, you are | |
| | | | | | a T carrier, and you stop | |
| | | | | | consuming for a significant | |
| | | | | | period of time, you may have | |
| | | | | | higher levels of THC for an | |
| | | | | | extended period of time. You | |
| | | | | | may find withdrawal easier | |
| | | | | | than the other genotypes (C | |
| | | | | | carrier) because you may | |
| | | | | | excrete THC more slowly. It | |
| | | | | | is important to note that this | |
| | | | | | polymorphism is only one of | |
| | | | | | many contributing factors to | |
| | | | | | withdrawal experiences. | |
| 3-01-A | AKT1 | rs1130233 | Response to | COGNITIVE | This polymorphism is | 22290123 |
| | | | Cannabinoids | | associated with the risk of | |
| | | | | | psychotomimetic (psychosis- | |
| | | | | | like) effects after consuming | |
| | | | | | THC. The risk is especially | |
| | | | | | increased by an interaction | |
| | | | | | with the SLC6A3 3′UTR | |
| | | | | | VNTR polymorphism (9 | |
| | | | | | repeat allele). It is important | |
| | | | | | to note that this | |
| | | | | | polymorphism is only one of | |
| | | | | | many contributing factors to | |
| | | | | | this side effect. | |
| 4-01-A | AKT1 | rs1130233 | Response to | COGNITIVE | Impaired motor control can | 25065544 |
| | | | Cannabinoids | | be caused by THC, but this | |
| | | | | | effect is modulated by this | |
| | | | | | polymorphism. It is | |
| | | | | | important to note that this | |
| | | | | | polymorphism is only one of | |
| | | | | | many contributing factors to | |
| | | | | | this profile. | |
| 5-01-2AL | AKT1 | rs2494732 | Response to | COGNITIVE | This polymorphism may | 22831980 |
| | | | Cannabinoids | | confer a significantly | |
| | | | | | elevated risk of a psychotic | |
| | | | | | disorder for those who use | |
| | | | | | cannabis -- and when some | |
| | | | | | previous psychosis of some | |
| | | | | | sort is present, cannabis may | |
| | | | | | dramatically increase further | |
| | | | | | psychotic experiences. | |
| | | | | | However, this polymorphism | |
| | | | | | is only one of many | |
| | | | | | contributing factors to this | |
| | | | | | profile. | |
| 6-01-2AL | AKT1 | rs2494732 | Response to | COGNITIVE | Cognitive functioning, | 21775978 |
| | | | Cannabinoids | | including attention and | |
| | | | | | accuracy, are adversely | |
| | | | | | affected by THC. This effect | |
| | | | | | may be modified by this | |
| | | | | | AKT1 polymorphisms and | |
| | | | | | by interactions with | |
| | | | | | psychosis. It is important to | |
| | | | | | note that polymorphism is | |
| | | | | | only one of many | |
| | | | | | contributing factors to this | |
| | | | | | profile. | |
| 7-01-2AL | AKT1 | rs2494732 | Response to | COGNITIVE | THC can cause acute | 26882038 |
| | | | Cannabinoids | | psychotomimetic (psychosis- | |
| | | | | | like) effects in some people | |
| | | | | | and this response is | |
| | | | | | modulated by this AKT1 | |
| | | | | | polymorphism. It is | |
| | | | | | important to note that this | |
| | | | | | polymorphism is only one of | |
| | | | | | many contributing factors to | |
| | | | | | this profile. | |
| 8-01-2AL | AKT1 | rs2494732 | Response to | COGNITIVE | This polymorphism was | 27336035; |
| | | | Cannabinoids | | associated with greater risk | PMC4849451 |
| | | | | | of developing psychosis in | |
| | | | | | cannabis risers. It is | |
| | | | | | important to note that this | |
| | | | | | polymorphism is only one of | |
| | | | | | many contributing factors to | |
| | | | | | this profile. | |
| 9-01-ALG | BDNF | rs6265 | Response to | RESPONSE | Cannabis use was associated | 21305693 |
| | | | Cannabinoids | | with earlier age of onset of a | |
| | | | | | psychotic disorder. In one | |
| | | | | | study, this effect was | |
| | | | | | stronger with a higher | |
| | | | | | frequency of cannabis use | |
| | | | | | and earlier age at first use. In | |
| | | | | | males, this effect occured | |
| | | | | | independent of BDNF | |
| | | | | | genotype. In females, the | |
| | | | | | effect of cannabis use on age | |
| | | | | | of onset of psychosis was | |
| | | | | | highly dependent on this | |
| | | | | | polymorphism. It is | |
| | | | | | important to note that this | |
| | | | | | polymorphism is only one of | |
| | | | | | many contributing factors to | |
| | | | | | this profile. | |
| | CNR1 | (rs202329, | Response to | COGNITIVE | Based on your TAG | 24445906 |
| | | rs1535255, | Cannabinoids | | haplotype of ACA, you may | |
| | | rs806379) | | | have a higher risk of | |
| | | | | | experiencing acute | |
| | | | | | psychomimetic effects after | |
| | | | | | consumption of THC. | |
| | | | | | [In exploring CNR1, AKT1, | |
| | | | | | BDNF and COMT genes | |
| | | | | | with psychotomimetic effects | |
| | | | | | when first using cannabis | |
| | | | | | (PEFU), Reserchers found a | |
| | | | | | significant association with a | |
| | | | | | functional haplotype block in | |
| | | | | | CNR1], (Overall | |
| | | | | | reccomendation include not | |
| | | | | | consuming alcohol and other | |
| | | | | | substances in parallel with | |
| | | | | | THC use). Consult a | |
| | | | | | specialist for a dose titration/ | |
| | | | | | adjustment. It is important | |
| | | | | | to note that this | |
| | | | | | polymorphism is only one of | |
| | | | | | many contributing factors to | |
| | | | | | this profile. | |
| 16-01- | CNR1 | rs1O49353 | Response to | COGNITIVE | This polymorphism was | roar.uel.ac.uk/4985/1/ |
| 2AFML | | | Cannabinoids | | associated with performance | Stephanie%20Marie%20Lynch.pdf |
| | | | | | on a test of executive | |
| | | | | | function (mental skills that | |
| | | | | | help you set and accomplish | |
| | | | | | goals) and sustained | |
| | | | | | attention in a combined | |
| | | | | | group of cannabis users and | |
| | | | | | non-users. It is important to | |
| | | | | | note that this is only one of | |
| | | | | | many factors determining on | |
| | | | | | how you sustain attention | |
| | | | | | and focus on activities. | |
| 17-01- | CNR1 | rs1406977 | Response to | COGNITIVE | This polymorphism was | 25139064; |
| AFM | | | Cannabinoids | | associated with performance | 27261878 |
| | | | | | on a working memory task in | |
| | | | | | cannabis users, but not in | |
| | | | | | non-cannabis users. It is | |
| | | | | | important to note that this is | |
| | | | | | only one of many factors | |
| | | | | | determining your working | |
| | | | | | memory profile. | |
| 26-01-2 | COMT | rs4680 | Response to | COGNITIVE | This polymorphism was | 21947654; |
| | | | Cannabinoids | | associated with the onset of | 23445265; |
| | | | | | psychotomimetic (psychosis- | 26464454; |
| | | | | | like) effects after consuming | 16936704 |
| | | | | | THC. However, this was not | |
| | | | | | confirmed by all studies. | |
| | | | | | Other studies found an | |
| | | | | | interacting effect of | |
| | | | | | childhood abuse or | |
| | | | | | psychosis-related cognitive | |
| | | | | | changes. It is important to | |
| | | | | | note that this polymorphism | |
| | | | | | is only one of many | |
| | | | | | contributing factors to this | |
| | | | | | profile. | |
| 27-01-2 | COMT | rs4680 | Response to | COGNITIVE | This polymorphism was | 23311613 |
| | | | Cannabinoids | | associated with the volume/ | |
| | | | | | neuroanatomical changes of | |
| | | | | | specific brain regions in | |
| | | | | | young males who are chronic | |
| | | | | | cannabis users. These | |
| | | | | | include the ventral caudate | |
| | | | | | nucleus (involved in | |
| | | | | | memory), the left amygdala | |
| | | | | | (involved in emotions such | |
| | | | | | as anxiety), prefrontal cortex, | |
| | | | | | neostriatum (caudate- | |
| | | | | | putamen), ACC and the | |
| | | | | | hippocampus-amygdala | |
| | | | | | complex (respectively tied | |
| | | | | | into short-term memory | |
| | | | | | emotions, moods, and other | |
| | | | | | functions related to | |
| | | | | | depression and anxiety). It is | |
| | | | | | important to note that this is | |
| | | | | | only one of many factors that | |
| | | | | | could be linked to anxiety | |
| | | | | | and other emotions. | |
| 28-01-2 | COMT | rs4680 | Response to | COGNITIVE | This polymorphism was | 26882038; |
| | | | Cannabinoids | | associated with memory and | 16936704; |
| | | | | | other cognitive impairments | 26464454; |
| | | | | | after consuming THC. It is | 23449176 |
| | | | | | important to note that this | |
| | | | | | polymorphism is only one of | |
| | | | | | many contributing factors to | |
| | | | | | this profile. | |
| 29-01-2 | COMT | rs4680 | Response to | RESPONSE | Individuals who carried high | 26950642 |
| | | | Cannabinoids | | function COMT and low- | |
| | | | | | function DRD4 7R alleles (a | |
| | | | | | combination expected to be | |
| | | | | | associated with higher risk) | |
| | | | | | showed more lifetime | |
| | | | | | cannabis abuse in a cohort of | |
| | | | | | women with binge-purge | |
| | | | | | eating disorders. | |
| 30-01- | CYP2C9 | rs1057910 | Response to | RESPONSE | This polymorphism was | 19005461 |
| 2AFMLG | | | Cannabinoids | | associated with metabolism | |
| | | | | | and pharmacokinetics of oral | |
| | | | | | THC. THC levels | |
| | | | | | immediately after pulmonary | |
| | | | | | administration (smoking, | |
| | | | | | vaporizing) are not highly | |
| | | | | | affected by the rate of | |
| | | | | | metabolism, so this | |
| | | | | | polymorphism probably has | |
| | | | | | little effect with these | |
| | | | | | methods. No effect on CBD | |
| | | | | | levels is expected since it is | |
| | | | | | not metabolized to a | |
| | | | | | significant extent by | |
| | | | | | CYP2C9. | |
| 31-01 | CYP3A4 | rs35599367 | Response to | RESPONSE | This polymorphism was | 23750331 |
| | | | Cannabinoids | | associated with activity of | |
| | | | | | the CYP3A4 enzyme, which | |
| | | | | | can metabolize both THC | |
| | | | | | and CBD. Although this | |
| | | | | | polymorphism has not | |
| | | | | | directly been associated with | |
| | | | | | cannabinoid metabolism, | |
| | | | | | clinical studies of the | |
| | | | | | CYP3A4 inhibitor | |
| | | | | | ketoconazole showed that it | |
| | | | | | boosts both THC and CBD | |
| | | | | | levels after oronmcosal | |
| | | | | | dosing by approximately 2- | |
| | | | | | fold. Therefore, it is likely | |
| | | | | | that this polymorphism | |
| | | | | | affects THC and CBD levels | |
| | | | | | after oral dosing. | |
| 32-01 | DAT1 | VNTR | Response to | COGNITIVE | Genetic Factors are a key | 29404409 |
| | | | Cannabinoids | | component in how your body | |
| | | | | | responds to THC and CBD | |
| | | | | | by way of absorption, | |
| | | | | | distribution, metabolism, and | |
| | | | | | excretion. The hippocampus | |
| | | | | | is a brain region associated | |
| | | | | | with learning, memory, and | |
| | | | | | emotions. Although cannabis | |
| | | | | | use was associated with a | |
| | | | | | decreased volume of the | |
| | | | | | hippocampus, the effect was | |
| | | | | | highly dependent on this | |
| | | | | | polymorphism. It is | |
| | | | | | important to note that this | |
| | | | | | polymorphism is only one of | |
| | | | | | many contributing factors to | |
| | | | | | this profile. | |
| 33-01 | DBH | rs1611115 | Response to | COGNITIVE | This polymorphism was | 26667034 |
| | | | Cannabinoids | | associated with impulsivity | |
| | | | | | after cannabis consumption | |
| | | | | | in a group of people that | |
| | | | | | regularly used cannabis and | |
| | | | | | cocaine. It is important to | |
| | | | | | note that this polymorphism | |
| | | | | | is only one of many | |
| | | | | | contributing factors to this | |
| | | | | | profile. | |
| 34-01- | DRD2 | rs1076560 | Response to | COGNITIVE | This polymorphism was | 27336035 |
| 2AFMLG | | | Cannabinoids | | associated with greater risk | |
| | | | | | of developing psychosis in | |
| | | | | | cannabis users. It is | |
| | | | | | important to note that this | |
| | | | | | polymorphism is only one of | |
| | | | | | many contributing factors to | |
| | | | | | this profile. | |
| 41-01 | FAAH | rs324420 | Response to | RESPONSE | This polymorphism was | 26106535 |
| | | | Cannabinoids | | associated with brain white | |
| | | | | | matter integrity in young | |
| | | | | | cannabis users. Lower white | |
| | | | | | matter integrity was linked to | |
| | | | | | apathy and depression. It is | |
| | | | | | important to note that this | |
| | | | | | polymorphism is only one of | |
| | | | | | many contributing factors to | |
| | | | | | this profile. | |
| 42-01 | MAPK14 | rs12199654 | Response to | COGNITIVE | This polymorphism was | 22850347 |
| | | | Cannabinoids | | associated with white matter | |
| | | | | | volume reduction in | |
| | | | | | cannabis-dependent | |
| | | | | | schizophrenic patients. | |
| | | | | | White matter is important for | |
| | | | | | cognition and executive | |
| | | | | | control (e.g. attention and | |
| | | | | | planning). It is important to | |
| | | | | | note that this polymorphism | |
| | | | | | is only one of many | |
| | | | | | contributing factors to this | |
| | | | | | profile. | |
| 46-01 | NRG1 | rs22153 3 | Response to | COGNITIVE | This polymorphism was | roar.uel.ac.uk/4985/1/ |
| | | | Cannabinoids | | associated with some | Stephanie%20Marie%20Lynch.pdf |
| | | | | | symptoms of schizotypal | |
| | | | | | personality. It is important to | |
| | | | | | note that this polymorphism | |
| | | | | | is only one of many | |
| | | | | | contributing factors to this | |
| | | | | | profile. | |
| 47-01 | NRG1 | rs7834206 | Response to | COGNITIVE | This polymorphism was | 20582876 |
| | | | Cannabinoids | | associated with deficits in | |
| | | | | | auditory information | |
| | | | | | processing after cannabis | |
| | | | | | consumption. Auditor | |
| | | | | | information processing | |
| | | | | | deficits are associated with | |
| | | | | | schizophrenia. It is important | |
| | | | | | to note that this | |
| | | | | | polymorphism is only one of | |
| | | | | | many contributing factors to | |
| | | | | | this profile. | |
| 50-01 | SLC6A3 | rs28363170 | Response to | COGNITIVE | This polymorphism was | 22290123 |
| | | | Cannabinoids | | associated with the risk of | |
| | | | | | psychotomimetic (psychosis- | |
| | | | | | like) effects after consuming | |
| | | | | | THC. The risk was | |
| | | | | | especially increased by an | |
| | | | | | interaction with the AKT1 | |
| | | | | | rs1130233 polymorphism. It | |
| | | | | | is important to note that this | |
| | | | | | polymorphism is only one of | |
| | | | | | many contributing factors to | |
| | | | | | this profile. | |
| 51-01 | SLC6A3 | rs28363170 | Response to | COGNITIVE | This polymorphism was | 22290123 |
| | | | Cannabinoids | | associated with the risk of | |
| | | | | | psychotomimetic (psvchosis- | |
| | | | | | like) effects after consuming | |
| | | | | | THC. There was a further | |
| | | | | | interaction of this | |
| | | | | | polymorphisms with the | |
| | | | | | AKT1 rs130233 | |
| | | | | | polymorphism. The | |
| | | | | | psychotomimetic effects of | |
| | | | | | THC may be especially | |
| | | | | | increased in subjects who | |
| | | | | | carry both the risk alleles. | |
| | | | | | Furthermore, this effect | |
| | | | | | involves an alteration in the | |
| | | | | | neural response to THC in | |
| | | | | | the dopamine-rich regions of | |
| | | | | | striatum and midbrain, | |
| | | | | | consistent w ith independent | |
| | | | | | evidence that the | |
| | | | | | psychotomimetic effects of | |
| | | | | | cannabis are mediated by | |
| | | | | | dopamine. It is important to | |
| | | | | | note that this polymorphism | |
| | | | | | is only one of many | |
| | | | | | contributing factors to this | |
| | | | | | profile. | |
| 52-01 | SLC6A4 | 5-HTTLPR | Response to | COGNITIVE | This polymorphism is | 26860734 |
| | | | Cannabinoids | | associated with the | |
| | | | | | development of anxiety | |
| | | | | | following cannabis use in | |
| | | | | | adolescents. Research | |
| | | | | | indicates that cannabis use is | |
| | | | | | associated with an increase | |
| | | | | | in symptoms of anxiety but | |
| | | | | | only in carriers of the short | |
| | | | | | allele of the 5-HTTLPR | |
| | | | | | polymorphism. It is | |
| | | | | | important to note that this | |
| | | | | | polymorphism is only one of | |
| | | | | | many contributing factors to | |
| | | | | | this profile. | |
| 53-01 | SLC6A4 | 5-HTTLPR | Response to | COGNITIVE | This polymorphism was | 20434316 |
| | | | Cannabinoids | | associated with psychotic | |
| | | | | | symptoms in patients with | |
| | | | | | bipolar disorder. The short | |
| | | | | | allele of the 5-HTTLPR | |
| | | | | | polymorphism of the 5-HTT | |
| | | | | | gene was associated with | |
| | | | | | psychotic symptoms when | |
| | | | | | when there was a | |
| | | | | | dependence or abuse of | |
| | | | | | cannabis. It is important to | |
| | | | | | note that this polymorphism | |
| | | | | | is only one of many | |
| | | | | | contributing factors to this | |
| | | | | | profile. | |
| 54-01 | SLC6A4 | 5-HTTLPR | Response to | COGNITIVE | This polymorphism was | 23449176 |
| | | | Cannabinoids | | associated with decision | |
| | | | | | making abilities in a manner | |
| | | | | | dependence on both | |
| | | | | | genotype and cannabis use. | |
| | | | | | Among youth with two | |
| | | | | | “short” alleles of the 5- | |
| | | | | | HTTLPR polymorphism, | |
| | | | | | decision making abilities | |
| | | | | | were significantly worse in | |
| | | | | | cannabis users. Decision | |
| | | | | | making ability was similar in | |
| | | | | | cannabis users and non-users | |
| | | | | | of other genotypes. It is | |
| | | | | | important to note that this | |
| | | | | | polymorphism is only one of | |
| | | | | | many contributing factors to | |
| | | | | | this profile. | |
| | CNR1 | (AAT)n | Physical | IMMUNE | This polymorphism was | 23306084; |
| | | microsatellite | Health & | | associated with Irritable | 24444427 |
| | | | Wellness | | Bowel Syndrome (IBS) and | |
| | | | | | severity of symptoms in | |
| | | | | | patients with IBS. Hence, | |
| | | | | | research supports the | |
| | | | | | hypothesis that cannabinoid | |
| | | | | | receptors may play a role in | |
| | | | | | control of colonic transit and | |
| | | | | | sensation in humans. It is | |
| | | | | | important to note that this | |
| | | | | | polymorphism is only one of | |
| | | | | | many contributing factors to | |
| | | | | | this profile. | |
| 20-01- | CNR1 | rs806366 | Physical | RELIEF | Migraines and its symptoms | 27762084 |
| AFM | | | Health & | | may be exacerbated by | |
| | | | Wellness | | recent stressful life events. | |
| | | | | | Tliis polymorphism was | |
| | | | | | associated with headache | |
| | | | | | with nausea only in those | |
| | | | | | people who had experienced | |
| | | | | | recent stressful events. It is | |
| | | | | | important to note that this is | |
| | | | | | only one of many factors | |
| | | | | | determining whether and | |
| | | | | | how you may experience | |
| | | | | | migraine symptoms. | |
| 39-01 | FAAH | rs324420 | Physical | RESPONSE | This polymorphism was | 26808012 |
| | | | Health & | | associated with cold pain | |
| | | | Wellness | | sensitivity and use of | |
| | | | | | postoperative analgesia. It is | |
| | | | | | important to note that this | |
| | | | | | polymorphism is only one of | |
| | | | | | many contributing factors to | |
| | | | | | this profile. | |
| 40-01 | FAAH | rs324420 | Physical | RESPONSE | Some opioid effects are | 25558980; |
| | | | Health & | | potentiated by cannabinoids, | 27977335 |
| | | | Wellness | | including the | |
| | | | | | endocannabinoid | |
| | | | | | anandamidc. This | |
| | | | | | polymorphism lias been | |
| | | | | | linked to the severity of | |
| | | | | | morphine side effects, such | |
| | | | | | as respiratory depression and | |
| | | | | | nausea/vomiting. in children | |
| | | | | | and adolescents following | |
| | | | | | surgery. It is important to | |
| | | | | | note that this polymorphism | |
| | | | | | is only one of many | |
| | | | | | contributing factors to this | |
| | | | | | profile. | |
| 11-01- | CNR1 | rs1049353 | Mental | COGNITIVE | This polymorphism was | 26717543 |
| 2AFML | | | Health & | | associated with signs of post- | |
| | | | Wellness | | traumatic threat symptoms. | |
| | | | | | In particular, minor An allele | |
| | | | | | carrier w ho also scored high | |
| | | | | | on a measure of early child | |
| | | | | | hood trauma reported greater | |
| | | | | | threat symptoms and | |
| | | | | | hypervigilance reactions. | |
| | | | | | The association depends on | |
| | | | | | the level of childhood | |
| | | | | | physical abuse experienced. | |
| | | | | | It is important to note that | |
| | | | | | this polymorphism is only | |
| | | | | | one of many factors | |
| | | | | | contributing to the | |
| | | | | | development these kind of | |
| | | | | | symptoms. | |
| 12-01- | CNR1 | rs1049353 | Mental | COGNITIVE | A polymorphism in the | 22826533 |
| 2AFML | | | Health & | | endocannabinoid system was | |
| | | | Wellness | | linked to the antidepressant | |
| | | | | | responses for a class of | |
| | | | | | antidepressants called | |
| | | | | | selective serotonin reuptake | |
| | | | | | inhibitors (SSRIs). | |
| | | | | | Specifically, the C/C | |
| | | | | | genotype of this CNR1 | |
| | | | | | polymorphism was | |
| | | | | | associated with a better | |
| | | | | | response to the | |
| | | | | | antidepressant citalopram/ | |
| | | | | | Celexa* in males. It is | |
| | | | | | important to note that this | |
| | | | | | polymorphism is only one of | |
| | | | | | many contributing factors to | |
| | | | | | this profile. | |
| 13-01- | CNR1 | rs1049353 | Mental | COGNITIVE | The endocannabinoid | 24980155 |
| 2AFML | | | Health & | | signaling system (ECS) helps | |
| | | | Wellness | | controls neural development, | |
| | | | | | particularly during | |
| | | | | | adolescence. The ECS is | |
| | | | | | vulnerable to disturbances | |
| | | | | | during this time (including | |
| | | | | | exposure to exogenous | |
| | | | | | cannabinoids) and | |
| | | | | | disturbances may lead to | |
| | | | | | impairments in self | |
| | | | | | control. In one study of | |
| | | | | | communities at risk for extra | |
| | | | | | stressors, this polymorphism | |
| | | | | | was linked to adolescent | |
| | | | | | impulsivity following early | |
| | | | | | psychosocial adversity. In | |
| | | | | | particular, early adversity is | |
| | | | | | linked to enhanced | |
| | | | | | impulsivity among | |
| | | | | | homozygous carriers of the | |
| | | | | | rs806379 A and the | |
| | | | | | rslO49353 T allele when | |
| | | | | | compared to homozygous | |
| | | | | | carriers of the respective | |
| | | | | | major allele. It is important | |
| | | | | | to note that this | |
| | | | | | polymorphism is only one of | |
| | | | | | many contributing factors to | |
| | | | | | this profile. | |
| 14-01- | CNR1 | rs1049353 | Mental | COGNITIVE | The theta wave is a type of | 25116250 |
| 2AFML | | | Health & | | brain wave measured by | |
| | | | Wellness | | EEG that is correlated with | |
| | | | | | working memory | |
| | | | | | performance. This | |
| | | | | | polymorphism was | |
| | | | | | associated with the | |
| | | | | | magnitude of the theta wave. | |
| | | | | | It is important to note that | |
| | | | | | this polymorphism is only | |
| | | | | | one of many contributing | |
| | | | | | factors to tlris profile. | |
| 15-01- | CNR1 | rs1049353 | Mental | COGNITIVE | Tliis polymorphism was | 26527537 |
| 2AFML | | | Health & | | associated with the | |
| | | | Wellness | | recognition of negative | |
| | | | | | emotions in adolescents and | |
| | | | | | the activation of associated | |
| | | | | | brain areas that process | |
| | | | | | emotional recognition. These | |
| | | | | | brain areas include the insula | |
| | | | | | and amygdala. Specifically, | |
| | | | | | adolescent C/C carriers of | |
| | | | | | this polymorphism had | |
| | | | | | greater insula and amygdala | |
| | | | | | activation of viewing angry | |
| | | | | | faces.This is part of a larger | |
| | | | | | body of research on how | |
| | | | | | complex social cues are | |
| | | | | | learned. Researchers note | |
| | | | | | that this overlaps with the | |
| | | | | | endocannabinoid system | |
| | | | | | modulating the mesolimbic | |
| | | | | | dopaminergic system. It is | |
| | | | | | important to note that this | |
| | | | | | polymorphism is only one of | |
| | | | | | many contributing factors to | |
| | | | | | tliis profile. | |
| 19-01- | CNR1 | rs2023239 | Mental | COGNITIVE | This polymorphism was | 26331953 |
| AFMG | | | Health & | | associated with depression in | |
| | | | Wellness | | a population of patients on | |
| | | | | | methadone maintenance. For | |
| | | | | | those with the C allele there | |
| | | | | | are some indications of a | |
| | | | | | protective role against major | |
| | | | | | depressive disorder (MDD). | |
| | | | | | This polymorphism is only | |
| | | | | | one of many factors | |
| | | | | | contributing to the | |
| | | | | | development of depression. | |
| 21-01- | CNR1 | rs806377 | Mental | COGNITIVE | In one study, it was found | 24690898 |
| AFLG | | | Health & | | that C allele carriers of this | |
| | | | Wellness | | CNR1 polymorphism have | |
| | | | | | an increased subjective | |
| | | | | | happiness levels, including | |
| | | | | | experiencing more overall | |
| | | | | | happiness in life and | |
| | | | | | experiencing greater positive | |
| | | | | | emotions after a positive | |
| | | | | | event. It is important to note | |
| | | | | | that this polymorphism is | |
| | | | | | only one of many factors | |
| | | | | | contributing to the | |
| | | | | | development of these kind of | |
| | | | | | outlooks/experiences. | |
| 23-01 | CNR1 | rs806379 | Mental | COGNITIVE | The endocannabinoid | 24980155 |
| | | | Health & | | signaling system (ECS) helps | |
| | | | Wellness | | control neural development, | |
| | | | | | particularly during | |
| | | | | | adolescence. The ECS is | |
| | | | | | vulnerable to disturbances | |
| | | | | | during this time (including | |
| | | | | | exposure to exogenous | |
| | | | | | cannabinoids) and | |
| | | | | | disturbances may lead to | |
| | | | | | impairments in self | |
| | | | | | control. In one study of | |
| | | | | | communities at risk for extra | |
| | | | | | stressors, this polymorphism | |
| | | | | | was linked to adolescent | |
| | | | | | impulsivity following early | |
| | | | | | psychosocial adversity. In | |
| | | | | | particular, early adversity is | |
| | | | | | linked to enhanced | |
| | | | | | impulsivity among | |
| | | | | | homozygous carriers of the | |
| | | | | | rs806379 A and the | |
| | | | | | rsl049353 T allele when | |
| | | | | | compared to homozygous | |
| | | | | | carriers of the respective | |
| | | | | | major allele. It is important | |
| | | | | | to note that this | |
| | | | | | polymorphism is only one of | |
| | | | | | many contributing factors to | |
| | | | | | this profile. | |
| 24-01 | CNR2 | rs2501432 | Mental | COGNITIVE | In a Japanese study that | 18991891 |
| | | | Health & | | compared both humans, this | |
| | | | Wellness | | polymorphism was | |
| | | | | | associated with higher | |
| | | | | | vulnerability to depression. It | |
| | | | | | is important to note that this | |
| | | | | | is only one of many factors if | |
| | | | | | and how you may experience | |
| | | | | | depressive behaviors. | |
| 25-01 | CNR2 | rs41311993 | Mental | COGNITIVE | Tliis polymorphism was | 21658778 |
| | | | Health & | | significantly associated with | |
| | | | Wellness | | the risk for developing | |
| | | | | | bipolar disorder (BD). It is | |
| | | | | | important to note that this is | |
| | | | | | only one of many factors | |
| | | | | | linked to tliis profile. | |
| 37-01 | FAAH | rs324420 | Mental | COGNITIVE | This polymorphism was | 19103437 |
| | | | Health & | | associated with threat-related | |
| | | | Wellness | | anxiety, reward-related | |
| | | | | | impulsivity, and activation of | |
| | | | | | associated brain areas (the | |
| | | | | | amygdala and ventral | |
| | | | | | striatum, respectively). It is | |
| | | | | | important to note that this | |
| | | | | | polymorphism is only one of | |
| | | | | | many contributing factors to | |
| | | | | | tliis profile. | |
| 38-01 | FAAH | rs324420 | Mental | REST | This polymorphism was | 27074158 |
| | | | Health & | | associated with poorer sleep | |
| | | | Wellness | | quality among young | |
| | | | | | cannabis users. Depressive | |
| | | | | | symptoms were identified as | |
| | | | | | a possible link between this | |
| | | | | | polymorphism and poor | |
| | | | | | sleep quality. It is important | |
| | | | | | to note that this | |
| | | | | | polymorphism is only one of | |
| | | | | | many contributing factors to | |
| | | | | | this profile. | |
| 1-01- | ABCB1 | rs1045642 | Drug | RESPONSE | This polymorphism was | 19625010 |
| AFMLG | | | Dependence | | associated with the risk of | |
| | | | | | cannabis dependence. This | |
| | | | | | may be due to effects on | |
| | | | | | brain penetration of THC or | |
| | | | | | effects on elimination of | |
| | | | | | THC from the body. It is | |
| | | | | | important to note that this | |
| | | | | | polymorphism is only one of | |
| | | | | | many contributing factors to | |
| | | | | | developing cannabis | |
| | | | | | dependence. | |
| 10-01- | CNR1 | rs1049353 | Drug | RESPONSE | This polymorphism had a | 19443135 |
| 2AFML | | | Dependence | | weak trend towards | |
| | | | | | association with cannabis | |
| | | | | | dependence symptoms in | |
| | | | | | young adults. This research | |
| | | | | | was also carried out in a | |
| | | | | | specific population of youth | |
| | | | | | with polysubstance | |
| | | | | | dependence and conduct | |
| | | | | | problems, and thus the | |
| | | | | | results may not be | |
| | | | | | generalizable to other groups | |
| | | | | | of youth or adults. It is | |
| | | | | | important to note that this is | |
| | | | | | only one of many factors | |
| | | | | | determining whether and | |
| | | | | | how cannabis dependence | |
| | | | | | occur. | |
| 18-01- | CNR1 | rs2023239 | Drug | RESPONSE | One research study linked | 18705688 |
| AFMG | | | Dependence | | this polymorphism to craving | |
| | | | | | for cannabis after 5 days of | |
| | | | | | abstinence in daily cannabis | |
| | | | | | smokers. Craving for a drug | |
| | | | | | is a warning sign for | |
| | | | | | psychological dependence. It | |
| | | | | | is important to note that this | |
| | | | | | polymorphism is only one of | |
| | | | | | many contributing factors to | |
| | | | | | dependency issues. | |
| 22-01 | CNR1 | rs806379 | Drag | COGNITIVE | Nicotine withdrawal can | 27453054 |
| | | | Dependence | | cause cognitive disruption, | |
| | | | | | which is partially mediated | |
| | | | | | by the endocannabinoid | |
| | | | | | system. This polymorphism | |
| | | | | | was associated with the | |
| | | | | | degree of cognitive | |
| | | | | | disruption during nicotine | |
| | | | | | withdrawal. Current research | |
| | | | | | suggests potential efficacy of | |
| | | | | | a pharmacotherapy approach | |
| | | | | | for smoking cessation among | |
| | | | | | individuals who exhibit | |
| | | | | | greater nicotine withdrawal- | |
| | | | | | related cognitive disruption. | |
| 35-01 | DRD2 | rsl800497 | Drug | COGNITIVE | Tliis polymorphism was | 26833047; |
| | | | Dependence | | associated with the risk of | 22536882 |
| | | | | | cannabis dependence. There | |
| | | | | | was also an interaction noted | |
| | | | | | with the CNR1 rs1049353 | |
| | | | | | polymorphism. It is | |
| | | | | | important to note that this | |
| | | | | | polymorphism is only one of | |
| | | | | | many contributing factors to | |
| | | | | | this profile. | |
| 36-01 | FAAH | rs324420 | Drug | RESPONSE | This polymorphism was | 24407958 |
| | | | Dependence | | associated with risky alcohol | |
| | | | | | use. which is a precursor to | |
| | | | | | more significant dependence | |
| | | | | | on alcohol. It is important to | |
| | | | | | note that this polymorphism | |
| | | | | | is only one of many | |
| | | | | | contributing factors to this | |
| | | | | | profile. | |
| 43-01 | MGLL | rs604300 | Drug | RESPONSE | This polymorphism was | 26595473 |
| | | | Dependence | | associated with cannabis | |
| | | | | | dependence in a maimer that | |
| | | | | | depended on the presence of | |
| | | | | | early childhood abuse. It is | |
| | | | | | important to note that this | |
| | | | | | polymorphism is only one of | |
| | | | | | many contributing factors to | |
| | | | | | this profile. | |
| 44-01 | NCAM1 | rs4471463 | Drug | RESPONSE | This polymorphism was | 27023175 |
| | | | Dependence | | associated with lifetime use | |
| | | | | | of cannabis. The specific | |
| | | | | | outcome of this study was | |
| | | | | | whether the subjects had ever | |
| | | | | | tried cannabis or had never | |
| | | | | | tried it. Although further | |
| | | | | | study is needed, this | |
| | | | | | polymorphism could also be | |
| | | | | | associated with cannabis | |
| | | | | | dependence. It is important | |
| | | | | | to note that this | |
| | | | | | polymorphism is only one of | |
| | | | | | many contributing factors to | |
| | | | | | this profile. | |
| 45-01 | NRG1 | rsl7664708 | Drug | RESPONSE | This polymorphism was | 22520967 |
| | | | Dependence | | associated with the risk of | |
| | | | | | cannabis dependence in a | |
| | | | | | group of African Americans. | |
| | | | | | This finding was not | |
| | | | | | replicated in European | |
| | | | | | Americans. It is important to | |
| | | | | | note that this polymorphism | |
| | | | | | is only one of many | |
| | | | | | contributing factors to this | |
| | | | | | profile. | |
| 48-01 | OPRM1 | rs1799971 | Drug | RESPONSE | This polymorphism was | 26392368 |
| | | | Dependence | | associated with a general risk | |
| | | | | | of substance abuse, including | |
| | | | | | cannabis dependence. It is | |
| | | | | | important to note that this | |
| | | | | | polymorphism is only one of | |
| | | | | | many contributing factors to | |
| | | | | | this profile. | |
| 49-01 | PENK | rs2609997 | Drug | RESPONSE | This polymorphism was | 22745721 |
| | | | Dependence | | associated with cannabis | |
| | | | | | dependence. This risk was | |
| | | | | | modified by the neuroticism | |
| | | | | | personality trait, which | |
| | | | | | describes a person's | |
| | | | | | propensity for experiencing | |
| | | | | | negative emotions. | |
| | | | | | (Limitations of the research | |
| | | | | | include that the sample was | |
| | | | | | drawn from a population | |
| | | | | | without significant | |
| | | | | | psychiatric comorbidity). It | |
| | | | | | is important to note that this | |
| | | | | | polymorphism is only one of | |
| | | | | | many contributing factors to | |
| | | | | | this profile. | |

| | | | | Suggestion - | | Suggestion - |
|---|---|---|---|---|---|---|
| | | Major | Minor | Homozygous MAJOR | Suggestion - | Homozygous MINOR |
| SNIP I.D. | Gene | Allele | Allele | Allele | Heterozygous | Allele |
| 2-01- | ABCB1 | C | T | Homozygous MAJOR | Heterozygous | Homozygous MINOR |
| AFMLG | | | | Alleles (C/C) | Alleles (C/T) | Alleles (T/T) |
| | | | | Research indicates you | Research indicates | Research indicates you |
| | | | | may have higher THC | you may have 2-fold | may have 2-fold lower |
| | | | | levels in your blood | lower THC levels in | blood THC levels after |
| | | | | after consuming THC | your blood after | consuming THC |
| | | | | relative to other | consuming THC | relative to people with |
| | | | | genotypes. | relative to people | the most common |
| | | | | | with the most | genotype. |
| | | | | | common genotype. | |
| 3-01-A | AKT1 | C | T | Homozygous MAJOR | Heterozygous | Homozygous MINOR |
| | | | | Alleles (C/C)Research | Alleles | Alleles (T/T)Research |
| | | | | indicates you may have | (C/T)Research | indicates you may have |
| | | | | an increased risk of | indicates you may | a lower risk of |
| | | | | experiencing acute | have a lower risk of | experiencing acute |
| | | | | psychotomimetic effects | experiencing acute | psychotomimetic effects |
| | | | | after consuming THC. | psychotomimetic | after consuming THC. |
| | | | | Your risk is highest if | effects after | |
| | | | | you also carry an | consuming THC. | |
| | | | | SLCA6 3′ UTR VNTR | | |
| | | | | 9R allele. Caution and | | |
| | | | | consultation with a | | |
| | | | | licensed medical | | |
| | | | | professional who | | |
| | | | | focuses on THC/CBD | | |
| | | | | titration/adjustments | | |
| | | | | and cessation.is | | |
| | | | | recommended to assess | | |
| | | | | your risks. | | |
| 4-01-A | AKT1 | C | T | Homozygous MAJOR | Heterozygous | Homozygous MINOR |
| | | | | Alleles (C/C) | Alleles (C/T) | Alleles (T/T) |
| | | | | Research indicates you | Research indicates | Research indicates you |
| | | | | may be less likely to | you may be less | may develop |
| | | | | develop coordination | likely to develop | coordination |
| | | | | impairment after | coordination | impairment after |
| | | | | consuming THC. | impairment after | consuming THC. Avoid |
| | | | | | consuming THC. | using alcohol when |
| | | | | | | consuming THC. |
| | | | | | | Careful titration of dose |
| | | | | | | under supervision a |
| | | | | | | licensed medical |
| | | | | | | professional who |
| | | | | | | focuses on THC/CBD |
| | | | | | | titration/adjustments |
| | | | | | | and cessation. |
| 5-01-2AL | AKT1 | T | C | Homozygous MAJOR | Heterozygous | Homozygous MINOR |
| | | | | Alleles (T/T)You may | Alleles (T/C)You | Alleles (C/C)You may |
| | | | | have a lower risk of | may have an | have an increased |
| | | | | developing a psychotic | increased likelihood | likelihood of a |
| | | | | disorder with cannabis | of a psychotic | psychotic disorder with |
| | | | | use relative to people | disorder with | cannabis use. This risk |
| | | | | with other genotypes. | cannabis use. This | may be especially |
| | | | | | risk is especially | elevated in daily |
| | | | | | elevated in daily | cannabis users. Caution |
| | | | | | cannabis users. | is warranted. |
| | | | | | Caution is warranted. | Consultation with a |
| | | | | | Consultation with a | licensed medical |
| | | | | | licensed medical | professional who |
| | | | | | professional who | focuses on THC/CBD |
| | | | | | focuses on THC & | titration is |
| | | | | | CBD titration is | recommended. |
| | | | | | recommended. | |
| 6-01-2AL | AKT1 | T | C | Homozygous MAJOR | Heterozygous | Homozygous MINOR |
| | | | | Alleles (T/T) | Alleles (T/C) | Alleles (C/C) |
| | | | | You may have a lower | You may have a | You may be at a higher |
| | | | | risk of memory and | lower risk of memory | risk of impairments in |
| | | | | attention impairments | and attention | memory and attention |
| | | | | after consuming THC. | impairments after | after consuming THC |
| | | | | | consuming THC. | relative to people with |
| | | | | | | other genotypes. |
| | | | | | | Responsible use is |
| | | | | | | warranted. |
| 7-01-2AL | AKT1 | T | C | Homozygous MAJOR | Heterozygous | Homozygous MINOR |
| | | | | Alleles (T/T)Research | Alleles (TC)You may | Alleles (C/C)You may |
| | | | | indicates you may have | have an increased | have an increased risk |
| | | | | a lower risk of | risk of experiencing | of experiencing acute |
| | | | | experiencing acute | acute | psychotomimetic effects |
| | | | | psychotomimetic effects | psychotomimetic | (psychosis-like) after |
| | | | | (psychosis-like) after | effects after | consuming THC. |
| | | | | consuming THC. | consuming THC. | Caution and |
| | | | | | Caution and | consultation with a |
| | | | | | consultation with a | specialist are |
| | | | | | specialist are | recommended to assess |
| | | | | | recommended to | your risks. |
| | | | | | assess your risks. | |
| 8-01-2AL | AKT1 | T | C | Homozygous MAJOR | Heterozygous | Homozygous MINOR |
| | | | | Alleles (T/T) | Alleles (T/C) | Alleles (C/C) |
| | | | | Research indicates that | If you are | If you are predisposed |
| | | | | THC may not increase | predisposed to | to developing |
| | | | | your risk of developing | developing psychosis, | psychosis, THC may |
| | | | | psychosis relative to | THC may increase | increase your risk. The |
| | | | | other genotypes. | your risk. The risk is | risk is highest with |
| | | | | | highest with more | more frequent use of |
| | | | | | frequent use of THC | THC and in those who |
| | | | | | and in those who also | also carry a DRD2 |
| | | | | | carry a DRD2 | rs107650 A allele. |
| | | | | | rs107650 A allele. | Caution and |
| | | | | | Caution and | consultation with a |
| | | | | | consultation with a | licensed medical |
| | | | | | licensed medical | professional who |
| | | | | | professional who | focuses on THC & CBD |
| | | | | | focuses on THC & | titration is |
| | | | | | CBD titration is | recommended to assess |
| | | | | | recommended to | your risks. |
| | | | | | assess your risks. | |
| 9-01-ALG | BDNF | G | A | Homozygous MAJOR | Heterozygous | Homozygous MINOR |
| | | | | Alleles (G/G)Research | Alleles (G/A)If you | Alleles (A/A)If you are |
| | | | | indicates If you are | are female who is | female who is |
| | | | | female who is | predisposed to | predisposed to |
| | | | | predisposed to | developing psychosis, | developing psychosis, |
| | | | | developing psychosis, | cannabis use may | cannabis use may result |
| | | | | cannabis use may not | result in a | in a significantly earlier |
| | | | | result in an earlier age | significantly earlier | age of onset of |
| | | | | of onset of psychosis. | age of onset of | psychosis. The effect of |
| | | | | | psychosis. The effect | cannabis depends on the |
| | | | | | of cannabis depends | extent of cannabis use |
| | | | | | on the extent of | and the age at which |
| | | | | | cannabis use and the | you start using it. Use |
| | | | | | age at which you start | with caution and consult |
| | | | | | using it. Use with | a specialist for a dose |
| | | | | | caution and consult a | titration. |
| | | | | | specialist for a dose | |
| | | | | | titration. | |
| | CNR1 | | | Based on your TAG | Based on your TAG | |
| | | | | haplotype of ACA, you | haplotype of AAA, | |
| | | | | may have a higher risk | you may have a lower | |
| | | | | of experiencing acute | risk of experiencing | |
| | | | | psychomimetic effects | acute psychomimetic | |
| | | | | after consumption of | effects after | |
| | | | | THC. Do not consume | consumption of THC. | |
| | | | | alcohol and other | | |
| | | | | substances in parallel | | |
| | | | | with THC use. Consult | | |
| | | | | a specialist for a dose | | |
| | | | | titration. | | |
| 16-01- | CNR1 | C | T | Homozygous MAJOR | Heterozygous | Homozygous MINOR |
| 2AFML | | | | Alleles (C/C)You may | Alleles (C/T)You | Alleles (T/T)You may |
| | | | | have greater ability in | may have an elevated | have an elevated risk of |
| | | | | sustained attention | risk of impairments | impairments in |
| | | | | relative to people with | in sustained attention | sustained attention with |
| | | | | other genotypes. | with cannabis use | cannabis use relative to |
| | | | | | relative to people | people with the most |
| | | | | | with the most | common genotype. |
| | | | | | common genotype. | Consume responsibly. |
| | | | | | Consume | |
| | | | | | responsibly. | |
| 17-01- | CNR1 | T | C | Homozygous MAJOR | Heterozygous | Homozygous MINOR |
| AFM | | | | Alleles (T/T) | Alleles (T/C) | Alleles (C/C) |
| | | | | You may be less likely | You may be more | You may be more likely |
| | | | | to experience working | likely to experience | to experience working |
| | | | | memory impairments | working memory | memory impairments |
| | | | | after use of THC. | impairments after use | after use of THC. |
| | | | | | of THC. Careful dose | Careful dose titration is |
| | | | | | titration is warranted. | warranted. Please |
| | | | | | Please consult a | consult a specialist. |
| | | | | | specialist. | |
| 26-01-2 | COMT | G | A | Homozygous MAJOR | Heterozygous | Homozygous MINOR |
| | | | | Alleles (G/G) | Alleles (G/A) | Alleles (A/A) |
| | | | | You may be more likely | You may be more | You may be less likely |
| | | | | to experience | likely to experience | to experience |
| | | | | psychotomimetic effects | psychotomimetic | psychotomimetic effects |
| | | | | after consuming THC. | effects after | after consuming THC |
| | | | | Although there are other | consuming THC. | relative to people with |
| | | | | predisposing factors, | Although there are | other genotypes. |
| | | | | caution is warranted | other predisposing | However, there are |
| | | | | upon THC | factors, caution is | other predisposing |
| | | | | consumption. | warranted upon THC | factors. |
| | | | | | consumption. | |
| 27-01-2 | COMT | G | A | Homozygous MAJOR | Heterozygous | Homozygous MINOR |
| | | | | Alleles (G/G)You may | Alleles (G/A)You | Alleles (AA)You may |
| | | | | be at increased risk of | may be at increased | have a decreased risk of |
| | | | | brain volume changes | risk of brain volume | brain volume changes |
| | | | | following cannabis use | changes following | following cannabis use |
| | | | | that are linked to | cannabis use that are | that are linked to |
| | | | | alterations in emotions | linked to alterations | alterations in emotions |
| | | | | and memory. | in emotions and | and memory. |
| | | | | | memory. | |
| 28-01-2 | COMT | G | A | Homozygous MAJOR | Heterozygous | Homozygous MINOR |
| | | | | Alleles (G/G) | Alleles (G/A) | Alleles (A/A) |
| | | | | You may experience | You may experience | You may experience |
| | | | | greater cognitive | greater cognitive | less cognitive |
| | | | | impairment after | impairment after | impairment after |
| | | | | consuming THC | consuming THC | consuming THC |
| | | | | relative to people with | relative to people | relative to people with |
| | | | | other genotypes. | with the AA | other genotypes. |
| | | | | Caution and careful | genotype. Caution | Caution and careful |
| | | | | titration of THC is | and careful titration | titration of THC is still |
| | | | | highly recommended. | of THC is highly | recommended. |
| | | | | | recommended. | |
| 29-01-2 | COMT | G | A | Homozygous MAJOR | Heterozygous | Homozygous MINOR |
| | | | | Alleles (G/G)You may | Alleles (G/A)You | Alleles (A/A)You may |
| | | | | have a higher risk for | may have a relatively | have a relatively lower |
| | | | | dependence on cannabis | lower risk to develop | risk to develop |
| | | | | and associated cravings | dependence on | dependence on cannabis |
| | | | | relative to other | cannabis relative to | relative to people with |
| | | | | genoty pes. Caution and | people with the most | the most common |
| | | | | consultation with a | common genotype. | genotype. |
| | | | | specialist is | | |
| | | | | recommended. | | |
| 30-01- | CYP2C9 | A | C | Homozy gous MAJOR | Heterozygous | Homozygous MINOR |
| 2AFMLG | | | | Alleles (A/A) | Alleles (A/C) | Alleles (C/C) |
| | | | | As you do not have a | You may feel the | You may feel the effects |
| | | | | version of CYP2C9 | effects of oral THC | of oral THC more |
| | | | | with reduced activity, | more strongly or find | strongly or find that the |
| | | | | you can start oral THC | that the effects last | effects last longer |
| | | | | at a typical dose. | longer relative to | relative to people with |
| | | | | However, this is not the | people with the most | the most common |
| | | | | only factor affecting | common genotype. It | genotype. It is |
| | | | | sensitivity to THC and | is recommended that | recommended that the |
| | | | | careful dose titration is | the first time you use | first time you use oral |
| | | | | still recommended. | oral THC, you start at | THC, you start at 30% |
| | | | | | 60% of the standard | of the standard dose. No |
| | | | | | dose. No change in | change in THC dose is |
| | | | | | THC dose is needed | needed for pulmonary |
| | | | | | for pulmonary | administration. |
| | | | | | administration. | |
| 31-01 | CYP3A4 | C | T | Homozygous MAJOR | Heterozygous | Homozygous MINOR |
| | | | | Alleles (C/C)As you | Alleles (C/T)You | Alleles (T/T)You may |
| | | | | have the ty pical version | may feel the effects | feel the effects of oral |
| | | | | of CYP3A4, you may | of oral THC more | THC more strongly or |
| | | | | start oral THC or CBD | strongly or find that | find that the effects last |
| | | | | at a normal dose. | the effects last longer | longer relative to other |
| | | | | However, this is not the | relative to other | people. It is |
| | | | | only factor affecting | people. It is | recommended that the |
| | | | | sensitivitity to THC. | recommended that | first time you use oral |
| | | | | | the first time you use | THC. you start at 50% |
| | | | | | oral THC. you start at | of the standard dose. No |
| | | | | | 75% of the standard | change in dose is |
| | | | | | dose. No change in | needed for pulmonary |
| | | | | | THC dose is needed | administration. |
| | | | | | for pulmonary | |
| | | | | | administration. | |
| 32-01 | DAT1 | | | You may be at risk for | This genotype was | This genotype was not |
| | | | | reduced hippocampal | not associated with | associated with reduced |
| | | | | volume with cannabis | reduced hippocampal | hippocampal volume in |
| | | | | use. This could lead to | volume in cannabis | cannabis users. |
| | | | | long- and short-term | users. | |
| | | | | memory impairments | | |
| | | | | and altered emotions. | | |
| | | | | Responsible use and | | |
| | | | | consultation with a | | |
| | | | | specialist is warranted. | | |
| 33-01 | DBH | C | T | Homozygous MAJOR | Heterozygous | Homozygous MINOR |
| | | | | Alleles (C/C)This | Alleles (C/T)You | Alleles (T/T)You may |
| | | | | genotype was not | may be more likely to | be more likely to act |
| | | | | associated with | act impulsively after | impulsively after |
| | | | | increased impulsivity | consuming cannabis. | consuming cannabis. |
| | | | | after cannabis use. | Responsible use is | Responsible use is |
| | | | | | warranted. | warranted. |
| 34-01- | DRD2 | C | A | Homozygous MAJOR | Heterozygous | Homozygous MINOR |
| 2AFMLG | | | | Alleles (C/C) | Alleles (A/C) | Alleles (A/A) |
| | | | | THC may not increase | If you are | If you are predisposed |
| | | | | your risk of developing | predisposed to | to developing |
| | | | | psychosis relative to | developing psychosis, | psychosis, THC may |
| | | | | other genotypes. | THC may increase | increase your risk. The |
| | | | | | your risk. The risk is | risk is highest with |
| | | | | | highest with more | more frequent use of |
| | | | | | frequent use of THC | THC and in those who |
| | | | | | and in those who also | also carry an AKT1 |
| | | | | | carry an AKT1 | rs2494732 C allele. |
| | | | | | rs2494732 C allele. | Caution and |
| | | | | | Caution and | consultation with a |
| | | | | | consultation with a | specialist are |
| | | | | | specialist are | recommended to assess |
| | | | | | recommended to | your risks. |
| | | | | | assess your risks. | |
| 41-01 | FAAH | C | A | Homozygous MAJOR | Heterozygous | Homozygous MINOR |
| | | | | Alleles (C/C) | Alleles (C/A) | Alleles (A/A) |
| | | | | You may be more likely | You may have less | You may have less risk |
| | | | | relative to other | risk for decreased | for decreased white |
| | | | | genotypes to experience | white matter integrity | matter integrity with |
| | | | | decreased white matter | with cannabis use | cannabis use. Cautious |
| | | | | integrity with cannabis | relative. Cautious use | use is warranted. |
| | | | | use at a young age. | is warranted. | |
| | | | | Careful dose titration is | | |
| | | | | warranted. Please | | |
| | | | | consult a specialist. | | |
| 42-01 | MAPK14 | A | G | Homozygous MAJOR | Heterozygous | Homozygous MINOR |
| | | | | Alleles (A/A)You may | Alleles (A/G)Your | Alleles (G/G)Your |
| | | | | have a higher risk of | genotype may not | genotype may not |
| | | | | decreased white matter | confer risk of | confer risk of decreased |
| | | | | brain volume from | decreased white | white matter volume |
| | | | | cannabis use. This could | matter volume from | from cannabis use. |
| | | | | impair your cognition. | cannabis use. | Cautious use is |
| | | | | Use with caution and | Cautious use is | warranted. |
| | | | | consult a specialist for a | warranted. | |
| | | | | dose titration. | | |
| 46-01 | NRG1 | T | C | Homozygous MAJOR | Heterozygous | Homozygous MINOR |
| | | | | Alleles (T/T) | Alleles (T/C) | Alleles (C/C) |
| | | | | You may have a lower | You may have a | You may have a higher |
| | | | | risk of experiencing | lower risk of | risk of experiencing |
| | | | | psychomimetic effects | experiencing | psychomimetic effects |
| | | | | following cannabis use | psychomimetic | following cannabis use |
| | | | | relative to people with | effects following | relative to people with |
| | | | | the (T/T) genotype. | cannabis. | the other genotypes. |
| | | | | | | Consume responsibly |
| | | | | | | and consult a specialist |
| | | | | | | for guidance. |
| 47-01 | NRG1 | C | A | Homozygous MAJOR | Heterozygous | Homozygous MINOR |
| | | | | Alleles (C/C) | Alleles (C/A) | Alleles (A/A) |
| | | | | You may be less likely | You may be more | You may be more likely |
| | | | | to develop | likely to develop | to develop |
| | | | | psychomimetic effects | psychomimetic | psychomimetic effects |
| | | | | after cannabis | effects after cannabis | after cannabis |
| | | | | consumption relative to | consumption. | consumption. |
| | | | | people with other | Responsible | Responsible |
| | | | | genotypes. Responsible | consumption and a | consumption and a |
| | | | | consumption and a | titration with a help | titration with a help of a |
| | | | | titration with a help of a | of a specialist is | specialist is warranted. |
| | | | | specialist is warranted. | warranted. | |
| 50-01 | SLC6A3 | 10R | 9R | You may have a lower | You may have an | You may have an |
| | | | | risk of experiencing | increased risk of | increased risk of |
| | | | | acute psychotomimetic | experiencing acute | experiencing acute |
| | | | | effects after consuming | psychotomimetic | psychotomimetic effects |
| | | | | THC. | effects after | after consuming THC. |
| | | | | | consuming THC. | Your risk is highest if |
| | | | | | Your risk is highest if | you also have an AKT1 |
| | | | | | you also have an | C/C genotype. Caution |
| | | | | | AKT1 C/C genotype. | and consultation with a |
| | | | | | Caution and | specialist are |
| | | | | | consultation with a | recommended to assess |
| | | | | | specialist are | your risks and prevent |
| | | | | | recommended to | them. |
| | | | | | assess your risks and | |
| | | | | | prevent them. | |
| 51-01 | SLC6A3 | 10R | 9R | You may have a lower | You may have a | You may have a higher |
| | | | | risk of impulsivity after | higher risk of | risk of impulsivity after |
| | | | | consuming cannabis | impulsivity after | consuming cannabis |
| | | | | compared to other | consuming cannabis | relative to the most |
| | | | | genotypes. | relative to the most | common genotype. |
| | | | | | common genotype. | |
| 52-01 | SLC6A4 | L′ | S′ | Adolescents may be at | Adolescents with this | Adolescents with this |
| | | | | increased risk of | genotype may be at | genotype may be at |
| | | | | developing anxiety | increased risk of | increased risk of |
| | | | | following cannabis use, | developing anxiety | developing anxiety |
| | | | | but this genotype was | following cannabis | following cannabis use. |
| | | | | not associated with | use. Cannabis should | Cannabis should only |
| | | | | extra risk. Cannabis | only be used | be used responsibly |
| | | | | should only be used | responsibly once | once legal age is |
| | | | | responsibly once legal | legal age is attained. | attained. |
| | | | | age is attained. | | |
| 53-01 | SLC6A4 | L′ | S′ | Your genotype was not | Please be advised that | Please be advised that if |
| | | | | associated with | if you or your close | you or your close blood |
| | | | | heightened risk of | blood related | related relatives have |
| | | | | psychotomimetic | relatives have bipolar | bipolar disorder, you |
| | | | | symptoms in patients | disorder, you may be | may be at a higher risk |
| | | | | with bipolar disorder. | at a higher risk of | of psychotic effects |
| | | | | | psychotic effects with | with cannabis |
| | | | | | cannabis | consumption\. Careful |
| | | | | | consumption\. | titration of THC dose |
| | | | | | Careful titration of | and a consultation with |
| | | | | | THC dose and a | a specialist is suggested. |
| | | | | | consultation with a | |
| | | | | | specialist is | |
| | | | | | suggested. | |
| 54-01 | SLC6A4 | L′ | S′ | You may be less likely | You may be less | You may be more likely |
| | | | | to experience a decrease | likely to experience a | to experience a decrease |
| | | | | in decision making | decrease in decision | in decision making |
| | | | | ability as a result of | making ability as a | ability as a result of |
| | | | | using cannabis. | result of using | using cannabis |
| | | | | | cannabis. | compared to people of |
| | | | | | | other genotypes. |
| | | | | | | Caution is warranted. |
| | CNR1 | | | Based on your genotype | Based on your | Based on your genotype |
| | | | | of <10/<10, you may | genotype of <10/>10, | of >10/>10, you may |
| | | | | have a lower risk of | you may have an | have an elevated risk of |
| | | | | developing IBS relative | elevated risk of | developing IBS. If you |
| | | | | to people of other | developing IBS. A | have IBS or develop it |
| | | | | genotypes. | specialist should be | in the future, you may |
| | | | | | consulted for THC | have more severe |
| | | | | | dose titration. | symptoms than patients |
| | | | | | | with a different |
| | | | | | | genotype. A specialist |
| | | | | | | should be consulted for |
| | | | | | | THC dose titration. |
| 20-01- | CNR1 | T | C | Homozygous MAJOR | Heterozygous | Homozygous MINOR |
| AFM | | | | Alleles (T/T)You may | Alleles (T/C)This | Alleles (C/C)This |
| | | | | be more likely to | genotype does not | genotype does not |
| | | | | experience headache | appear to confer | appear to confer greater |
| | | | | with nausea after a | greater risk of | risk of experiencing |
| | | | | stressful event in vour | experiencing | headache with nausea |
| | | | | life. Use THC | headache with nausea | after recent stressful life |
| | | | | responsibly and consult | after recent stressful | events. |
| | | | | a specialist to titrate a | life events. | |
| | | | | dose appropriately to | | |
| | | | | your condition. | | |
| 39-01 | FAAH | C | A | Homozygous MAJOR | Heterozygous | Homozygous MINOR |
| | | | | Alleles (C/C) | Alleles (C/A) | Alleles (A/A) |
| | | | | You may have higher | You may have higher | You may have |
| | | | | cold pain sensitivity and | cold pain sensitivity | significantly lower cold |
| | | | | more need for analgesia | and more need for | pain sensitivity and less |
| | | | | during periods of acute | analgesia during | need for analgesia |
| | | | | pain, such as after an | periods of acute pain, | during periods of acute |
| | | | | operation. | such as after an | pain, such as after an |
| | | | | | operation. | operation. |
| 40-01 | FAAH | C | A | Homozygous MAJOR | Heterozygous | Homozygous MINOR |
| | | | | Alleles (C/C) | Alleles (C/A) | Alleles (A/A) |
| | | | | You may have a lower | You may have a | You may have a higher |
| | | | | risk experiencing side | higher risk of | risk of experiencing |
| | | | | effects from opioids | experiencing side | side effects from |
| | | | | relative to people with | effects from opioids | opioids relative to |
| | | | | other genotypes. | relative to people | people with the most |
| | | | | | with the most | common genotype. If |
| | | | | | common genotype. If | you will be receiving |
| | | | | | you will be receiving | opioids, consider |
| | | | | | opioids, consider | informing your |
| | | | | | informing your | physician of a potential |
| | | | | | physician of a | predisposition to |
| | | | | | potential | respiratory depression |
| | | | | | predisposition to | and nausea/vomiting. |
| | | | | | respiratory | |
| | | | | | depression and | |
| | | | | | nausea/vomiting. | |
| 11-01- | CNR1 | C | T | Homozygous MAJOR | Heterozygous | Homozygous MINOR |
| 2AFML | | | | Alleles (C/C)Research | Alleles (C/T)If you | Alleles (T/T)If you |
| | | | | indicates you that if you | have experienced | have experienced |
| | | | | have experienced | childhood physical | childhood physical |
| | | | | childhood physical | abuse, you may have | abuse, you may have a |
| | | | | abuse, you may have | a lower risk of | lower risk of |
| | | | | increased risk of | posttraumatic threat | posttraumatic threat |
| | | | | posttraumatic threat | symptoms relative to | symptoms relative to |
| | | | | symptoms relative to | people with the most | people with the most |
| | | | | people with other | common genotype. | common genotype. |
| | | | | genotypes. Please | | |
| | | | | consult a specialist if | | |
| | | | | symptoms are present. | | |
| 12-01- | CNR1 | C | T | Homozygous MAJOR | Heterozygous | Homozygous MINOR |
| 2AFML | | | | Alleles (C/C) | Alleles (C/T) | Alleles (T/T) |
| | | | | Research indicates that | Research indicates | Research indicates that |
| | | | | if you are a male patient | that if you are a male | if you are a male patient |
| | | | | with depression, you | patient with | with depression, you |
| | | | | may respond better to | depression, you may | may have decreased |
| | | | | treatment with | have a decreased | likelihood of responding |
| | | | | antidepressants such as | likelihood of | to antidepressants such |
| | | | | citalopram. Consult a | responding to | as citalopram/Celexa. |
| | | | | specialist prior to | antidepressants such | Consult a specialist |
| | | | | cannabinoid use or | as citalopram/ | prior to cannabinoid use |
| | | | | making any change in | Celexa*. Consult a | or making any change |
| | | | | antidepressant | specialist prior to | in antidepressant |
| | | | | treatment. | cannabinoid use or | treatment. |
| | | | | | making any change in | |
| | | | | | antidepressant | |
| | | | | | treatment. | |
| 13-01- | CNR1 | C | T | Homozygous MAJOR | Heterozygous | Homozygous MINOR |
| 2AFML | | | | Alleles (C/C)Research | Alleles | Alleles (T/T)Research |
| | | | | indicates adolescents | (C/T)Research | indicates that |
| | | | | who experienced early | indicates adolescents | adolescents who |
| | | | | psychosocial adversity | who experienced | experienced early |
| | | | | may not have an | early psychosocial | psychosocial adversity |
| | | | | elevated risk of | adversity may have a | may have a higher of |
| | | | | impulsive behavior. | slightly higher of risk | risk of impulsive |
| | | | | | of impulsive | behavior. Consult a |
| | | | | | behavior. Consult a | specialist for |
| | | | | | specialist for | cannabinoid dose |
| | | | | | cannabinoid dose | titration. |
| | | | | | titration. | |
| 14-01- | CNR1 | C | T | Homozygous MAJOR | Heterozygous | Homozygous MINOR |
| 2AFML | | | | Alleles (C/C) | Alleles (C/T) | Alleles (T/T) |
| | | | | You may have lower | Research indicates | Research indicates you |
| | | | | resting state EEG theta | you may have a | may have a higher |
| | | | | power. This may be | higher resting state | resting state EEG theta |
| | | | | correlated with lower | EEG theta power | power relative to the |
| | | | | working memory | relative to the most | most common |
| | | | | performance. | common genotype. | genotype. This may be |
| | | | | | This may be | correlated with better |
| | | | | | correlated with better | working memory. |
| | | | | | working memory. | |
| 15-01- | CNR1 | C | T | Homozygous MAJOR | Heterozygous | Homozygous MINOR |
| 2AFML | | | | Alleles (C/C)You may | Alleles (C/T)You | Alleles (T/T)You may |
| | | | | have greater amygdala | may have less | have less amygdala |
| | | | | and insula activation | amygdala and insula | activation upon viewing |
| | | | | upon viewing angry | activation upon | angry faces similar |
| | | | | faces or similar stimuli. | viewing angry faces | stimuli. |
| | | | | | or similar stimuli. | |
| 19-01- | CNR1 | T | c | Homozygous MAJOR | Heterozygous | Homozygous MINOR |
| AFMG | | | | Alleles (T/T) | Alleles (T/C) | Alleles (C/C) |
| | | | | You may be more likely | You may be less | You may be less likely |
| | | | | to experience | likely to experience | to experience |
| | | | | depression. Use THC | depression relative to | depression relative to |
| | | | | with caution as it could | people with the most | people with the most |
| | | | | potentiate your | common genotype. | common genotype. |
| | | | | symptoms Consult a | Caution with use of | Caution with use of |
| | | | | specialist for a careful | THC is still | THC is still |
| | | | | dose titration / | recommended. | recommended. |
| | | | | adjustment. | | |
| 21-01- | CNR1 | T | C | Homozygous MAJOR | Heterozygous | Homozygous MINOR |
| AFLG | | | | Alleles (T/T)Rcscarch | Alleles | Alleles (C/C)Research |
| | | | | indicates that you may | (T/C)Research | indicates you may |
| | | | | experience more overall | indicates that you | experience more overall |
| | | | | happiness in life and | may experience more | happiness in life and |
| | | | | experience greater | overall happiness in | experience greater |
| | | | | positive emotions after | life and experience | positive emotions after |
| | | | | a positive event. In one | greater positive | a positive event, noting |
| | | | | study, C allele of CNR1 | emotions after a | that long term |
| | | | | was significantly | positive event, noting | sociological factors |
| | | | | associated with an | that long term | affect the momentary |
| | | | | increased subjective | sociological factors | emotional state. |
| | | | | happiness level noting | affect the momentary | |
| | | | | that long term | emotional state. | |
| | | | | sociological factors | | |
| | | | | affect the momentary | | |
| | | | | emotional state. | | |
| 23-01 | CNR1 | A | T | Homozygous MAJOR | Heterozygous | Homozygous MINOR |
| | | | | Alleles (A/A)Research | Alleles | Alleles (T/T)Research |
| | | | | indicates that | (A/T)Research | indicates that |
| | | | | adolescents who | indicates that | adolescents who |
| | | | | experienced early | adolescents who have | experienced early |
| | | | | psychosocial adversity | experienced early | psychosocial adversity |
| | | | | may have a higher risk | psychosocial | may not have an |
| | | | | of impulsive behavior. | adversity may have a | elevated risk of |
| | | | | Consult a specialist for | slightly higher of risk | impulsive behavior. |
| | | | | cannabinoid dose | of impulsive | |
| | | | | titration. | behavior. Consult a | |
| | | | | | specialist for | |
| | | | | | cannabinoid dose | |
| | | | | | titration. | |
| 24-01 | CNR2 | C | T | Homozygous MAJOR | Heterozygous | Homozygous MINOR |
| | | | | Alleles (C/C) | Alleles (C/T) | Alleles (T/T) |
| | | | | Your risk of depression | You may have a | You may have a higher |
| | | | | may be lower relative to | higher than average | than average risk of |
| | | | | people with other | risk of developing | developing depression. |
| | | | | genotypes. This | depression. Watch for | Watch for symptoms of |
| | | | | polymorphism is only | symptoms of | depression and consider |
| | | | | factor in the risk for | depression and | consulting with a |
| | | | | developing depression. | consider consulting | specialist if these |
| | | | | | with a specialist if | symptoms are |
| | | | | | these symptoms are | interfering with your |
| | | | | | interfering with your | daily life. |
| | | | | | daily life. | |
| 25-01 | CNR2 | G | T | Homozygous MAJOR | Heterozygous | Homozygous MINOR |
| | | | | Alleles (G/G)You may | Alleles (G/T)You | Alleles (T/T)You may |
| | | | | have a lower risk of | may have a higher | have a higher risk of |
| | | | | developing bipolar | risk of developing | developing bipolar |
| | | | | disorder relative to | bipolar disorder | disorder relative to |
| | | | | people of other | relative to people of | people of the most |
| | | | | genotypes. | the most common | common genotype. |
| | | | | | genotype. | |
| 37-01 | FAAH | C | A | Homozygous MAJOR | Heterozygous | Homozygous MINOR |
| | | | | Alleles (C/C) | Alleles (C/A) | Alleles (A/A) |
| | | | | You may have greater | You may have | You may have reduced |
| | | | | anxiety (and associated | reduced anxiety (and | anxiety (and associated |
| | | | | activation of the | associated activation | activation of the |
| | | | | amygdala) in a | of the amygdala) in a | amygdala) in a |
| | | | | threatening situation | threatening situation | threatening situation |
| | | | | relative to other | relative to the most | relative to the most |
| | | | | genotypes. However, | common genotype. | common genotype. |
| | | | | you may have decreased | Hoyvever, you may | Hoyvever, you may have |
| | | | | reward-related | have increased | increased reward- |
| | | | | impulsivity. | reyvard-related | related impulsivity (and |
| | | | | | impulsivity (and | associated activation of |
| | | | | | associated activation | the ventral striatum). |
| | | | | | of the ventral | |
| | | | | | striatum). | |
| 38-01 | FAAH | C | A | Homozygous MAJOR | Heterozygous | Homozygous MINOR |
| | | | | Alleles (C/C) | Alleles (C/A) | Alleles (A/A) |
| | | | | You may be at | This polymorphism | This polymorphism |
| | | | | increased risk of poor | does not appear to | does not appear to |
| | | | | sleep quality. Consume | increase your risk of | increase your risk of |
| | | | | THC responsibly and | poor sleep. | poor sleep. |
| | | | | consult a specialist for | | |
| | | | | an optimal dose | | |
| | | | | titration. | | |
| 1-01- | ABCB1 | C | T | Homozygous MAJOR | Heterozygous | Homozygous MINOR |
| AFMLG | | | | Alleles (C/C)Research | Alleles | Alleles (T/T) Research |
| | | | | indicates you may have | (C/T)Research | indicates you may have |
| | | | | a higher risk of cannabis | indicates you may | an even less cannabis |
| | | | | dependence. Consider | have a higher risk of | dependence relative to |
| | | | | consulting with a | cannabis dependence. | other genotypes |
| | | | | medical professional | Consider consulting | combinations. Your |
| | | | | with experience in THC | with a medical | genotype at this |
| | | | | & CBD dose titration. | professional with | polymorphism does not |
| | | | | | experience in THC & | suggest a specific type |
| | | | | | CBD dose titration. | of cannabis product you |
| | | | | | | should consume. Your |
| | | | | | | choice of cannabis |
| | | | | | | product can be driven |
| | | | | | | by any symptoms you |
| | | | | | | may have. However, |
| | | | | | | thoroughly check all |
| | | | | | | polymorphisms in this |
| | | | | | | report for areas where |
| | | | | | | you may have |
| | | | | | | heightened risks. |
| 10-01- | CNR1 | C | T | Homozygous MAJOR | Heterozygous | Homozygous MINOR |
| 2AFML | | | | Alleles (C/C) | Alleles (C/T) | Alleles (T/T) |
| | | | | You may have a higher | You may have a | You may have a lower |
| | | | | risk of developing | higher risk of | risk of developing |
| | | | | symptoms of cannabis | developing symptoms | symptoms of cannabis |
| | | | | dependence. Please | of cannabis | dependence relative to |
| | | | | consume responsibly | dependence. Please | people with the most |
| | | | | and consult with a | consume responsibly | common genotype. |
| | | | | specialist for guidance. | and consult with a | |
| | | | | | specialist for | |
| | | | | | guidance. | |
| 18-01- | CNR1 | T | C | Homozygous MAJOR | Heterozygous | Homozygous MINOR |
| AFMG | | | | Alleles (T/T)Research | Alleles | Alleles (C/C)Research |
| | | | | indicates you that if you | (T/C)Research | indicates you that if you |
| | | | | are a regular cannabis | indicates you if you | are a regular cannabis |
| | | | | user, you may | are a regular cannabis | user, you may |
| | | | | experience less cravings | user, you may | experience stronger |
| | | | | and withdrawal after | experience stronger | cravings and withdrawal |
| | | | | stopping relative to the | cravings and | after stopping relative to |
| | | | | other genotypes. If there | withdrawal after | people with the most |
| | | | | are indications you are | stopping relative to | common genotype. If |
| | | | | dependent on cannabis | people with the most | you feel that you are |
| | | | | and have trouble | common genotype. If | dependent on cannabis |
| | | | | stopping, it is | you feel that you are | and have trouble |
| | | | | recommended that you | dependent on | stopping, it is |
| | | | | consult with a licensed | cannabis and have | recommended that you |
| | | | | medical professional | trouble stopping, it is | consult with a licensed |
| | | | | who focuses on THC/ | recommended that | medical professional |
| | | | | CBD titration and | you consult with a | who focuses on THC & |
| | | | | cessation. | licensed medical | CBD titration and |
| | | | | | professional who | cessation. |
| | | | | | focuses on THC/ | |
| | | | | | CBD titration/ | |
| | | | | | adjustments and | |
| | | | | | cessation. | |
| 22-01 | CNR1 | A | T | Homozygous MAJOR | Heterozygous | Homozygous MINOR |
| | | | | Alleles (A/A) | Alleles (A/T) | Alleles (T/T) |
| | | | | Research indicates that | Research indicates | Research indicates that |
| | | | | based on your genotype, | that based on your | based on your genotype, |
| | | | | you may exhibit greater | genotype, you may | you may exhibit less |
| | | | | nicotine withdrawal- | exhibit less nicotine | nicotine withdrawal- |
| | | | | related cognitive | withdrawal-related | related cognitive |
| | | | | dismption. | cognitive dismption. | dismption. |
| 35-01 | DRD2 | G | A | Homozy gous MAJOR | Heterozygous | Homozygous MINOR |
| | | | | Alleles (G/G)You may | Alleles (G/A)You | Alleles (A/A)You may |
| | | | | have decreased | may have decreased | have a greater |
| | | | | likelihood of developing | likelihood of | likelihood of developing |
| | | | | cannabis dependence. | developing cannabis | cannabis dependence, |
| | | | | | dependence. | with associated cravings |
| | | | | | | and mood changes. |
| | | | | | | Avoid mixing alcohol |
| | | | | | | and cannabis. Consult a |
| | | | | | | specialist for dose |
| | | | | | | titration. |
| 36-01 | FAAH | C | A | Homozy gous MAJOR | Heterozygous | Homozygous MINOR |
| | | | | Alleles (C/C) | Alleles (C/A) | Alleles (A/A) |
| | | | | You may be | You may have a | You may have a lower |
| | | | | significantly more likely | lower risk of | risk of engaging in risky |
| | | | | to use alcohol in a risky | engaging in risky | alcohol use. |
| | | | | fashion. Use THC | alcohol use. | |
| | | | | responsibly and don't | | |
| | | | | combine with alcohol | | |
| | | | | use. | | |
| 43-01 | MGLL | G | A | Homozygous MAJOR | Heterozygous | Homozygous MINOR |
| | | | | Alleles (G/G) | Alleles (G/A) | Alleles (A/A) |
| | | | | Although vour genotype | You may have | You may have |
| | | | | may be protective | increased risk of | increased risk of |
| | | | | against cannabis | cannabis dependence | cannabis dependence |
| | | | | dependence, this | independent of early | independent of early |
| | | | | protective effect appears | childhood stress or | childhood stress or |
| | | | | to be absent if you have | abuse. Caution and | abuse. Caution and |
| | | | | experienced early | responsible use is | responsible use is |
| | | | | childhood stress or | warranted. | warranted. |
| | | | | abuse. Cautious use is | | |
| | | | | warranted. Consult a | | |
| | | | | specialist for a dose | | |
| | | | | titration. | | |
| 44-01 | NCAM1 | T | C | Homozygous MAJOR | Heterozygous | Homozygous MINOR |
| | | | | Alleles (T/T)You may | Alleles (T/C)You | Alleles (C/C)You may |
| | | | | be less likely to ever | may have a greater | have a greater |
| | | | | consume cannabis | likelihood of using | likelihood of using |
| | | | | relative to people with | cannabis relative to | cannabis relative to |
| | | | | other genotypes. | people with the most | people with the most |
| | | | | | common genotype. | common genotype. |
| | | | | | Although not yet | Although not yet |
| | | | | | directly studied, this | directly studied, this |
| | | | | | may extend to a | may extend to a greater |
| | | | | | greater likelihood of | likelihood of cannabis |
| | | | | | cannabis dependence. | dependence. Consume |
| | | | | | Consume | responsibly. |
| | | | | | responsibly. | |
| 45-01 | NRG1 | C | T | Homozygous MAJOR | Heterozygous | Homozygous MINOR |
| | | | | Alleles (C/C) | Alleles (C/T) | Alleles (T/T) |
| | | | | If you are African | If you are African | If you are African |
| | | | | American, you may | American, you may | American, you may |
| | | | | have a lower risk of | have a higher risk of | have a higher risk of |
| | | | | cannabis dependence in | cannabis dependence | cannabis dependence |
| | | | | comparison to the other | relative to the most | relative to the most |
| | | | | genotypes. | common genotype. | common genotype. |
| 48-01 | OPRM1 | A | G | Homozygous MAJOR | Heterozygous | Homozygous MINOR |
| | | | | Alleles (A/A) | Alleles (A/G) | Alleles (G/G) |
| | | | | You may have a slightly | You may have a | You may have a slightly |
| | | | | higher risk of substance | slightly lower risk of | lower risk of substance |
| | | | | abuse, including | substance abuse, | abuse, including |
| | | | | cannabis dependence, | including cannabis | cannabis dependence, |
| | | | | relative to the other | dependence, relative | relative to the most |
| | | | | genotypes. | to the most common | common genotype. |
| | | | | | genotype. | |
| 49-01 | PENK | T | C | Homozygous MAJOR | Heterozygous | Homozygous MINOR |
| | | | | Alleles (T/T)You may | Alleles (T/C)You | Alleles (C/C)You may |
| | | | | be less likely to develop | may be more likely to | be more likely to |
| | | | | symptoms of cannabis | develop symptoms of | develop symptoms of |
| | | | | dependence relative to | cannabis dependence | cannabis dependence |
| | | | | people with other | relative to people | relative to people with |
| | | | | genotypes. | with the most | the most common |
| | | | | | common genotype. | genotype. This risk may |
| | | | | | This risk may be | be increased further if |
| | | | | | increased further if | you have high levels of |
| | | | | | you have high levels | neuroticism. Consume |
| | | | | | of neuroticism. | THC responsibly and |
| | | | | | Consume THC | consult a specialist for |
| | | | | | responsibly and | guidance. |
| | | | | | consult a specialist | |
| | | | | | for guidance. | |

All-allele disclaimer: Your choice of cannabis product can be driven by any symptoms you may have. However, thoroughly check all polymorphisms in this report for areas where you may have heightened risks.
Non-risk Allele disclaimer: Your genotype at this polymorphism does not suggest a specific type of cannabis.

1. A formulation comprising:
a cannabidiol (CBD) and a tetrahydrocannabinol (THC), wherein the formulation has a CBD:THC ratio from about 50:1 to about 1:50; a primary terpene; and a secondary terpene.

2. The formulation of claim 1, wherein the primary terpene is pinene and the secondary terpene is terpineol.

3. The formulation of claim 2, comprising 5% pinene and 3% terpineol, wherein the CBD:THC ratio is 4:1.

4. The formulation of claim 1, wherein the primary terpene is limonene and the secondary terpene is linalool.

5. The formulation of claim 4, comprising 5% limonene and 3% linalool, wherein the CBD:THC ratio is 1:1.

6. The formulation of claim 1, wherein the primary terpene is limonene and the secondary terpene is pinene.

7. The formulation of claim 6, comprising 5% limonene and 3% pinene, wherein the CBD:THC ratio is 1:2.

8. The formulation of claim 1, wherein the primary terpene is linalool and the secondary terpene is borneol.

9. The formulation of claim 8, comprising 5% linalool and 1% borneol, wherein the CBD:THC ratio is 4:1.

10. The formulation of claim 1, wherein the primary terpene is beta-caryophyllene and the secondary terpene is myrcene.

11. The formulation of claim 10, comprising 5% beta-caryophyllene and 3% myrcene, wherein the CBD:THC ratio is 1:1.

12. The formulation of claim 1, wherein the primary terpene is beta-caryophyllene and the secondary terpene is limonene.

13. The formulation of claim 12, comprising 5% beta-caryophyllene and 3% limonene, wherein the CBD:THC ratio is 1:2.

14. The formulation of claim 1, comprising branched-chain amino acids, L-glutamine, piperine, magnesium stearate, MCC, and silicon dioxide, wherein the CBD:THC ratio is about 1:2.

15. The formulation of claim 1, wherein the formulation comprises less than about 25%, 20%, 15%, 10%, 5%, 4%, 3%, or 2% terpenes.

16. The formulation of claim 1, wherein the formulation further comprises cannabigerol (CBG), cannabinol (CBN), cannabidvarin (CBDV), cannabidiolic acid (CBDA), tetrahydrocannabinolic acid (THCA), or tetrahydrocannabivarin (THCV).

17. The formulation of claim 1, wherein the formulation further comprises black pepper, branched-chain amino acids (BCAA), cayenne, cedarwood, chamomile, coconut oil, geranium, ginger, ginger oil, glutamine, guava, juniper berry, lavender, lemon, lemon oil, lemongrass, lime, lime oil, orange, orange oil, mango, marjoram, menthol, mint, mint oil, peppermint, peppermint oil, piperine, geranium, rosemary, sandalwood, or tangerine.

18. A method for a treating ADHD, autism, high energy, anxiety, hyperactivity, stress, depression, mood disorders, fatigue, low energy, cancer, pain, inflammation, or auto-immune disorders, comprising administering a pharmaceutically effective amount of the formulation of claim 1.
